SEQUENCE LISTING (4 sequences)

SEQ ID NO: 1 - protein, 15 aa, synthetic construct
QLLFNKTKSVEFTFC

SEQ ID NO: 2 - protein, 15 aa, synthetic construct
  MOD_RES 1: note = pyroglutamate
XLLFNKTKSVEFTFC

SEQ ID NO: 3 - protein, 15 aa, synthetic construct
  MOD_RES 1: note = pyroglutamate
  MOD_RES 6: note = stable isotope
XLLFNKTKSVEFTFC

SEQ ID NO: 4 - protein, 341 aa, synthetic construct
MRSGHHHHHHGSASAWPEEKNYHQPAILNSSALRQIAEGTSISEMWQNDLQPLLIERYPGSPGSYAARQHIMQRIQRLQADWVLEIDTFLSQTPEGERSFSNIISTLNPTAKRHLVLACHYDSKYFSHWNNRVFVGATDSAVPCAMMLELARALDKKLLSLKTVSDSKPDLSLQLIFFDGEEAFLHWSPQDSLYGSRHLAAKMASTPHPPGARGTSQLHGMDLLVLLDLIGAPNPTFPNFFPNSARWFERLQAIEHELHELGLLKDHSLEGRYFQNYSYGGVIQDDHIPFLRRGVPVLHLIPSPFPEVWHTMDDNEENLDESTIDNLNKILQVFVLEYLHL

(19) United States
(12) Patent Application Publication
(10) Pub. No.: US 2025/0066329 A1
(43) Pub. Date: Feb. 27, 2025

(54) PIPERIDINYLPYRIDINYLCARBONITRILE DERIVATIVES AS INHIBITORS OF GLUTAMINYL-PEPTIDE CYCLOTRANSFERASE AND GLUTAMINYL-PEPTIDE CYCLOTRANSFERASE LIKE PROTEIN

(71) Applicant: Boehringer Ingelheim International GmbH, Ingelheim am Rhein (DE)

(72) Inventors: Jens WILLWACHER, Biberach an der Riss (DE); Florian Paul Christian BINDER, Maselheim (DE); Georg DAHMANN, Biberach (DE); Sandra Ruth HANDSCHUH, Biberach an der Riss (DE); Sophia Astrid REINDL, Mittelbiberach (DE); James Young Soo YANG HAMILTON, Biberach (DE)

(21) Appl. No.: 18/789,814

(22) Filed: Jul. 31, 2024

(30) Foreign Application Priority Data
Aug. 14, 2023 (EP) 23191366.6

(51) Int. Cl. C07D 401/14; A61K 31/4545; A61K 31/501; A61K 45/06; C07D 471/04

(52) U.S. Cl. CPC C07D 401/14; A61K 31/4545; A61K 31/501; A61K 45/06; C07D 471/04

(57) ABSTRACT

The present disclosure provides certain piperidinylpyridinylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### TECHNICAL FIELD

The present disclosure provides certain piperidinylpyridinylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### BACKGROUND INFORMATION

Glutaminyl-peptide cyclotransferase (QPCT) and glutaminyl-peptide cyclotransferase-like protein (QPCTL) catalyze the intramolecular cyclization of N-terminal glutamine (Q) residues into pyroglutamic acid (pE) liberating ammonia [Stephan Schilling et al., “Identification of Human Glutaminyl Cyclase as a Metalloenzyme POTENT INHIBITION BY IMIDAZOLE DERIVATIVES AND HETEROCYCLIC CHELATORS,” Journal of Biological Chemistry 278, no. 50 (2003): 49773-79, https://doi.org/10.1074/jbc.m309077200; Holger Cynis et al., “Isolation of an Isoenzyme of Human Glutaminyl Cyclase: Retention in the Golgi Complex Suggests Involvement in the Protein Maturation Machinery,” Journal of Molecular Biology 379, no. 5 (2008): 966-80, https://doi.org/10.1016/j.jmb.2008.03.078; Anett Stephan et al., “Mammalian Glutaminyl Cyclases and Their Isoenzymes Have Identical Enzymatic Characteristics,” FEBS Journal 276, no. 22 (2009): 6522-36, https://doi.org/10.1111/j.1742-4658.2009.07337.x.]. While QPCT is a secreted protein, QPCTL is retained within the Golgi complex. Both enzymes share a high homology in the active site and similar catalytic specificity. Because of the high homology in the active site, inhibition of the active site blocks the enzymatic activity of both enzymes: QPCT and QPCTL. Hence the term “QPCT/L” describes both enzymes at once. Due to their different cellular localisation, differences in their relevance for modification of biological substrates have been reported. Known substrates of the intracellular QPCTL and/or extracellular QPCT are CD47 [Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.], different chemokines (like for example CCL2 and 7 or CX3CL1) [Rosa Barreira da Silva et al., “Loss of the Intracellular Enzyme QPCTL Limits Chemokine Function and Reshapes Myeloid Infiltration to Augment Tumor Immunity,” Nature Immunology 23, no. 4 (2022): 568-80, https://doi.org/10.1038/s41590-022-01153-x; Astrid Kehlen et al., “N-Terminal Pyroglutamate Formation in CX3CL1 Is Essential for Its Full Biologic Activity,” Bioscience Reports 37, no. 4 (2017): BSR20170712, https://doi.org/10.1042/bsr20170712.], Amyloid-b peptides [Cynis et al., “Isolation of an Isoenzyme of Human Glutaminyl Cyclase: Retention in the Golgi Complex Suggests Involvement in the Protein Maturation Machinery.” ] or hormones like TRH [Andreas Becker et al., “IsoQC (QPCTL) Knock-out Mice Suggest Differential Substrate Conversion by Glutaminyl Cyclase Isoenzymes,” Biological Chemistry 397, no. 1 (2016): 45-55, https://doi.org/10.1515/hsz-2015-0192.]. The modification of N-terminal glutamine to pyroglutamate on the substrates has functional consequences for the proteins and could impact different pathomechanisms in several diseases. CD47 is expressed on the cell surface of virtually all cells of the body, including apoptotic cells, senescent cells or cancer cells. [Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011]. The main ligand for CD47 is signal-regulatory protein alpha (SIRPα), an inhibitory transmembrane receptor present on myeloid cells, such as macrophages, monocytes, neutrophils, dendritic cells and others. QPCTL mediated N-terminal pyroglutamate modification on CD47 is required for SIRPα, binding [Deborah Hatherley et al., “Paired Receptor Specificity Explained by Structures of Signal Regulatory Proteins Alone and Complexed with CD47,” Molecular Cell 31, no. 2 (2008): 266-77, https://doi.org/10.1016/j.molcel.2008.05.026; Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.] This signaling axis induces a “Don't Eat Me Signal”, preventing engulfment of CD47 expressing cells by macrophages. Thus, high expression of CD47 is connected to the pathogenesis of cancer [Logtenberg et al., “Glutaminyl Cyclase Is an Enzymatic Modifier of the CD47-SIRPα Axis and a Target for Cancer Immunotherapy,” 2019; Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011.], COVID-19 [Katie-May McLaughlin et al., “A Potential Role of the CD47/SIRPalpha Axis in COVID-19 Pathogenesis,” Current Issues in Molecular Biology 43, no. 3 (2021): 1212-25, https://doi.org/10.3390/cimb43030086.], lung fibrosis [Gerlinde Wernig et al., “Unifying Mechanism for Different Fibrotic Diseases,” Proceedings of the National Academy of Sciences 114, no. 18 (2017): 4757-62, https://doi.org/10.1073/pnas.1621375114; Lu Cui et al., “Activation of JUN in Fibroblasts Promotes Pro-Fibrotic Programme and Modulates Protective Immunity,” Nature Communications 11, no. 1 (2020): 2795, https://doi.org/10.1038/s41467-020-16466-4.], systemic sclerosis [Wernig et al., “Unifying Mechanism for Different Fibrotic Diseases”; Tristan Lerbs et al., “CD47 Prevents the Elimination of Diseased Fibroblasts in Scleroderma,” JCI Insight 5, no. 16 (2020): e140458, https://doi.org/10.1172/jci.insight.140458.] and liver fibrosis [Taesik Gwag et al., “Anti-CD47 Antibody Treatment Attenuates Liver Inflammation and Fibrosis in Experimental Non-alcoholic Steatohepatitis Models,” Liver International 42, no. 4 (2022): 829-41, https://doi.org/10.1111/liv.15182.]. Since enhanced CD47 expression blocks the clearance of apoptotic cells, there is an accrual of apoptotic lung epithelial cells, leading to a pro-fibrotic stimulus and accelerating lung inflammation and -scaring [Alexandra L. McCubbrey and Jeffrey L. Curtis, “Efferocytosis and Lung Disease,” Chest 143, no. 6 (2013): 1750-57, https://doi.org/10.1378/chest.12-2413; Brennan D. Gerlach et al., “Efferocytosis Induces Macrophage Proliferation to Help Resolve Tissue Injury,” Cell Metabolism, 2021, https://doi.org/10.1016/j.cmet.2021.10.015.]. Since CD47 half-life and function is majorly dependent on QPCTL enzyme activity, QPCT and QPCTL inhibition could be a suitable mechanism as a treatment in lung fibrosis such as IPF or SSC-ILD [Lerbs et al., “CD47 Prevents the Elimination of Diseased Fibroblasts in Scleroderma.” ], alone or together with current standard of care in pulmonary fibrosis like Nintedanib [Luca Richeldi et al., “Efficacy and Safety of Nintedanib in Idiopathic Pulmonary Fibrosis,” The New England Journal of Medicine 370, no. 22 (2014): 2071-82, https://doi.org/10.1056/nejmoa1402584; Kevin R Flaherty et al., “Nintedanib in Progressive Fibrosing Interstitial Lung Diseases,” New England Journal of Medicine 381, no. 18 (2019): 1718-27, https://doi.org/10.1056/nejmoa1908681.] or future treatments like a PDE4 inhibitor [Luca Richeldi et al., “Trial of a Preferential Phosphodiesterase 4B Inhibitor for Idiopathic Pulmonary Fibrosis,” New England Journal of Medicine 386, no. 23 (2022): 2178-87, https://doi.org/10.1056/nejmoa2201737].

By expression of CD47, cancer cells can evade destruction by the immune system or evade immune surveillance, e.g. by evading phagocytosis by immune cells [Stephen B. Willingham et al., “The CD47-Signal Regulatory Protein Alpha (SIRPα) Interaction Is a Therapeutic Target for Human Solid Tumors,” Proceedings of the National Academy of Sciences 109, no. 17 (2012): 6662-67, https://doi.org/10.1073/pnas.1121623109].

In addition to CD47, chemokines, such as CCL2 and CX3CL1, have been identified as QPCTL and/or QPCT substrates [Holger Cynis et al., “The Isoenzyme of Glutaminyl Cyclase Is an Important Regulator of Monocyte Infiltration under Inflammatory Conditions,” EMBO Molecular Medicine 3, no. 9 (2011): 545-58, https://doi.org/10.1002/emmm.201100158]. The formation of the N-terminal pGlu was shown to increase in vivo activity, both by conferring resistance to aminopeptidases and by increasing its capacity to induce chemokine receptor signaling. Two main monocyte chemoattractants CCL2 and CCL7 are insensitive to DPP4-inactivation in vivo because of an intracellular mechanism of N-terminal cyclization mediated by the Golgi-associated enzyme QPCTL. It has been shown that QPCTL is a critical regulator of monocyte migration into solid tumors [Kaspar Bresser et al., “QPCTL Regulates Macrophage and Monocyte Abundance and Inflammatory Signatures in the Tumor Microenvironment,” Oncoimmunology 11, no. 1 (2022): 2049486, https://doi.org/10.1080/2162402x.2022.2049486; Rosa Barreira da Silva et al., “Loss of the Intracellular Enzyme QPCTL Limits Chemokine Function and Reshapes Myeloid Infiltration to Augment Tumor Immunity,” Nature Immunology, 2022, 1-13, https://doi.org/10.1038/s41590-022-01153-x]. Targeting of chemokines has long been pursued as a potential strategy for modulating cellular trafficking in different disease settings.

It is therefore desirable to provide potent QPCT/L inhibitors.

Jimenez-Sanchez, et al., Nature Chemical Biology, 2015, 11, 347-357, (hereinafter “J-S, NCB 2015”) discloses the human glutaminyl cyclase (hQC) inhibitors SEN177 and SEN180:

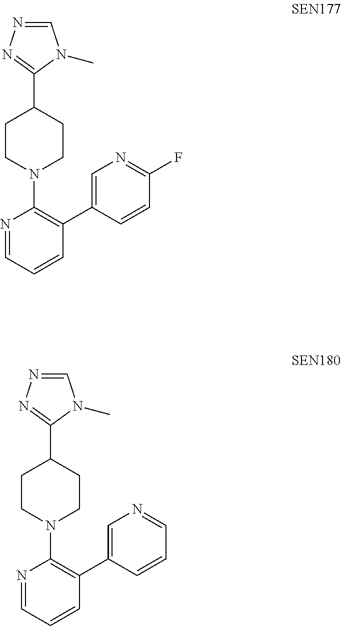 [SMILES: Cn1cnnc1C1CCN(c2ncccc2-c2ccc(F)nc2)CC1 ; Cn1cnnc1C1CCN(c2ncccc2-c2cccnc2)CC1]

SEN177 is disclosed therein (supplementary information) as having a IC₅₀ on isolated hQC of 53 nM and on isolated QPCTL of 13 nM. SEN180 is disclosed therein (supplementary information) as having a IC₅₀ on hQC of 170 nM and on QPCTL of 58 nM.

Pozzi, C, et al, Journal of Biological Inorganic Chemistry, 2018, 23, (8), 1219-1226, (hereinafter “P, JBIC 2018”), further discloses SEN177 and its binding mode within the hQC cavity. SEN177 is disclosed therein as having a Kᵢ on isolated hQC of 20 nM.

WO 2018/178384 discloses QPCTL inhibitors of the general formula A-B-D-E, which include examples 1094 and 1095 (Formula (XIIa) on page 123 and table on page 125):

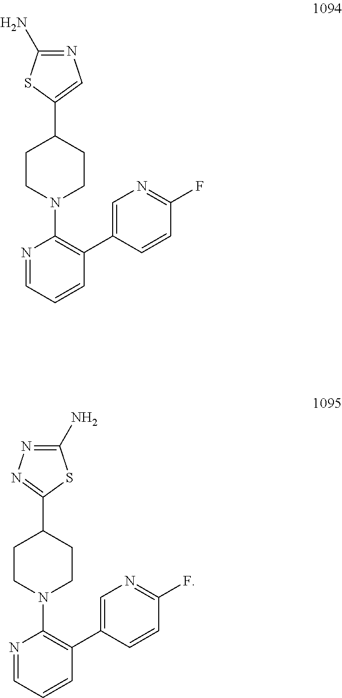

WO 2018/178384 does not disclose any biological data for examples 1094 or 1095.

WO 2022/086920 discloses QPCTL inhibitors of the general formula

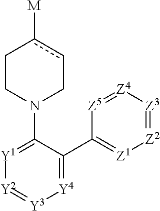

which include compounds 3 and 6:

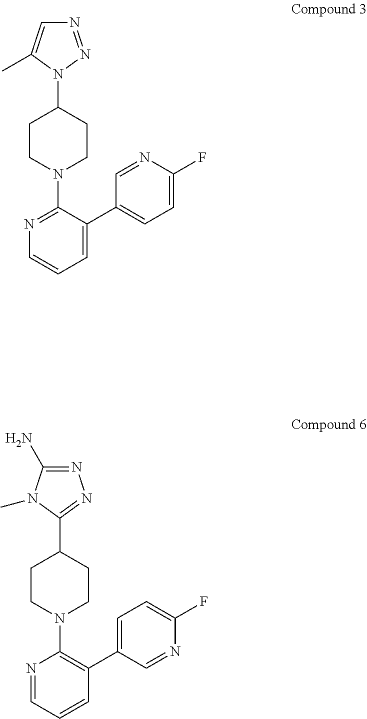 [SMILES: Cc1cnnn1C1CCN(c2ncccc2-c2ccc(F)nc2)CC1 ; Cn1c(N)nnc1C1CCN(c2ncccc2-c2ccc(F)nc2)CC1]

The chemical name of Compound 3 is disclosed in WO 2022/086920 as “1-(1-(6′-chloro-[3,3′-bipyridin]-2-yl)piperidin-4-yl)-1H-1,2,3-triazol-4-amine” which does not correspond to the chemical structure disclosed therein, but to an alternative structure in which the fluorine atom is replaced with chlorine:

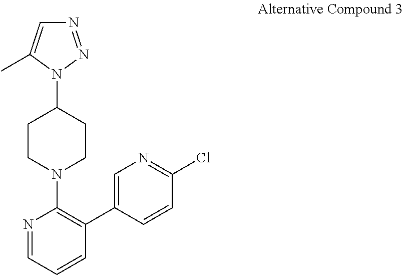 [SMILES: Cc1cnnn1C1CCN(c2ncccc2-c2ccc(Cl)nc2)CC1]

Compounds 3 (including alternative compound 3) and 6 in WO 2022/086920 are disclosed therein [00343] as having inhibitory activity on isolated QPCTL of IC₅₀<1 μM.

CN 114874186 discloses glutamine acyl cyclase isoenzyme inhibitors of the general formula

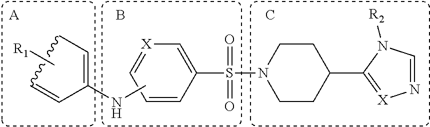

which include examples 21 and 23 (table on page 17):

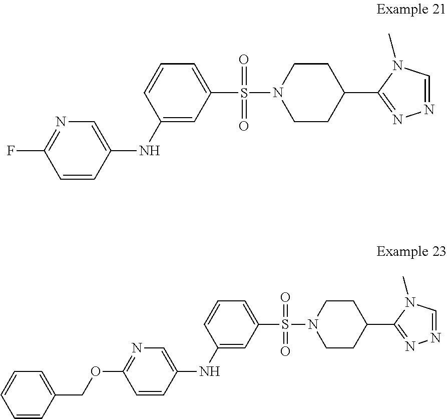 [SMILES: Cn1cnnc1C1CCN(S(=O)(=O)c2cccc(Nc3ccc(F)nc3)c2)CC1 ; Cn1cnnc1C1CCN(S(=O)(=O)c2cccc(Nc3ccc(OCc4ccccc4)nc3)c2)CC1]

IC₅₀'s are given in CN 114874186 for examples 21 and 23 as 29.22 nM and 11.26 nM respectively.

### DETAILED DESCRIPTION OF THE INVENTION

The present invention discloses novel piperidinylpyridinylcarbonitrile derivatives of formula (I)

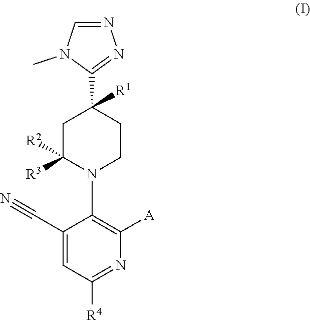

that are inhibitors of Glutaminyl-peptide cyclotransferase (QPCT) and glutaminyl-peptide cyclotransferase-like protein (QPCTL), possessing appropriate pharmacological and pharmacokinetic properties enabling their use as medicaments for the treatment of conditions and/or diseases treatable by inhibition of QPCT/L.

The compounds of the present invention may provide several advantages, such as enhanced potency, cellular potency, high metabolic and/or chemical stability, high selectivity, safety and tolerability, enhanced solubility, enhanced permeability, desirable plasma protein binding, enhanced bioavailability, suitable pharmacokinetic profiles, and the possibility to form stable salts.

### Compounds of the Invention

The present invention provides novel piperidinylpyridinylcarbonitrile derivatives that surprisingly, are potent inhibitors of QPCT and QPCTL (Assay A), as well as potent inhibitors of QPCT/L in cells relevant for, but not limited to, lung diseases or cancer, (Assay B).

Furthermore, the present novel piperidinylpyridinylcarbonitrile derivatives have appropriate membrane permeability and a low in vitro efflux (Assay C).

Consequently, compounds of the present invention are more viable for human use.

Compounds of the present invention differ structurally from SEN177 and SEN180 in J-S, NCB 2015, in that the pyridinyl ring attached to the piperidinyl ring contains a ring-nitrogen which is in a meta-position relative to the piperidinyl ring attachment position. Furthermore, a carbonitrile substituent is attached at the ortho-position to the piperidinyl ring attachment position of said pyridinyl ring. Still further, R¹, R² and R³ are not limited to hydrogen or methyl and A represents heterocyclic ring systems beyond pyridinyl. Still further, compounds of the present invention contain one or two chiral centres.

Compounds of the present invention differ structurally from examples 1094 and 1095 in WO 2018/178384 in that the pyridinyl ring attached to the piperidinyl ring contains a ring-nitrogen which is in a meta-position relative to the piperidinyl ring attachment position. Furthermore, a carbonitrile substituent is attached at the ortho-position to the piperidinyl ring attachment position of said pyridinyl ring. Still further, R¹, R² and R³ are not limited to hydrogen and A represents heterocyclic ring systems beyond pyridinyl. Still further, the 5-membered heterocyclic ring attached to the piperidinyl ring at the 4-position relative to the piperidinyl nitrogen is in example 1094 an aminothiazolyl ring and in example 1095 an aminothiadiazolyl ring whereas in compounds of the present invention it is a 3-substituted-4-methyl-4H-1,2,4-triazolyl ring. Still further, compounds of the present invention contain one or two chiral centres.

Compounds of the present invention differ structurally from compounds 3 (including alternative compound 3) and 6 in WO 2022/086920 in that the pyridinyl ring attached to the piperidinyl ring contains a ring-nitrogen which is in a meta-position relative to the piperidinyl ring attachment position. Furthermore, a carbonitrile substituent is attached at the ortho-position to the piperidinyl ring attachment position of said phenyl ring. Further, R¹, R² and R³ are not limited to hydrogen, and A represents heterocyclic ring systems beyond pyridinyl. Still further, the 5-membered heterocyclic ring “M” in the general formula of WO 2022/086920 is in compound 3 a regioisomer of the 3-substituted 4-methyl-4H-1,2,4-triazolyl ring in compounds of the present invention, and the 5-membered heterocyclic ring “M” in the general formula of WO 2022/086920 in compound 4 is a 3-substituted 4-methyl-4H-1,2,4-triazolyl ring as in compounds of the present invention but that it bears an amino group. Still further, compounds of the present invention contain one or two chiral centres.

Compounds of the present invention differ structurally from compounds 21 and 23 in CN114874186 in that the central sulfonamide moiety linking the piperidinyl ring to the phenyl ring is replaced by a direct bond. Furthermore, a carbonitrile substituent is attached at the ortho-position to the piperidinyl ring attachment position of said phenyl ring. Still further, compounds of the present invention contain one or two chiral centres.

These structural differences between compounds of the present invention and the prior art unexpectedly lead to a favourable combination of (i) potent inhibition of QPCT and QPCTL, (ii) potent inhibition of QPCT/L in cells relevant for, but not limited to, lung diseases or cancer, and (iii) appropriate membrane permeability and a low in vitro efflux.

Compounds of the invention are thus superior to those disclosed in the prior art in terms of the combination of the following parameters:
potent inhibition of QPCT and QPCTL (Assay A)
potent inhibition of QPCT/L in cells relevant for, but not limited to, lung diseases or cancer (Assay B)
appropriate membrane permeability and a low in vitro efflux (Assay C)

The present invention provides novel compounds according to formula (I)

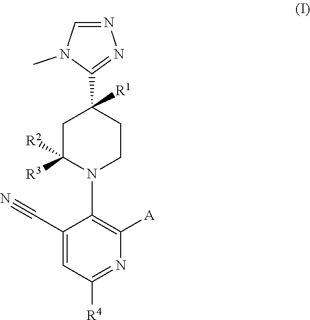

wherein
A is A1a which is a 6-membered mono-heteroaryl ring containing one or two heteroatom members selected from nitrogen;
or A is A1b which is a 9-membered fused bicyclic-heteroaryl ring containing one to four heteroatom members selected from nitrogen;
and wherein A is independently optionally substituted with one or two R⁵;
R¹ is selected from the group R1a, consisting of H and halo;
R² is selected from the group R2a, consisting of H, C₁₋₆-alkyl, F₁₋₉-fluoro-C₁₋₆-alkyl and C₃₋₆-cycloalkyl;
R³ is selected from the group R3a, consisting of H, C₁₋₆-alkyl, F₁₋₉-fluoro-C₁₋₆-alkyl and C₃₋₆-cycloalkyl;
with the proviso that R² and R³ are not both H;
or R² and R³ together with the carbon atom to which they are attached, form a cyclopropyl ring or a cyclobutyl ring; wherein said cyclopropyl or cyclobutyl ring is optionally substituted with one or two F;
R⁴ is selected from the group R4a, consisting of H, H₃C—O, HO—(H₃C)₂C, C₁₋₄-alkyl, C₃₋₆-cycloalkyl, F₁₋₉-fluoro-C₁₋₄-alkyl and F₁₋₉-fluoro-C₃₋₆-cycloalkyl;
R⁵ is selected from the group R5a, consisting of H, halo, C₁₋₆-alkyl and F₁₋₉-fluoro-C₁₋₄-alkyl;
or a salt thereof, particularly a pharmaceutically acceptable salt thereof.

Another embodiment of the present invention relates to a compound of formula (I), wherein A is A2 which is selected from the group consisting of

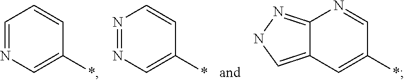

and substituents R¹, R², R³, R⁴ and R⁵ are defined as in any of the preceding embodiments.

Another embodiment of the present invention relates to a compound of formula (I), wherein A is A3 which is selected from the group consisting of

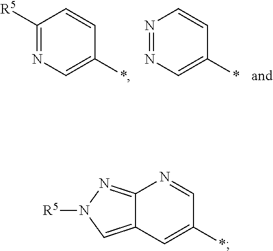

and substituents R¹, R², R³, R⁴ and R⁵ are defined as in any of the preceding embodiments.

Another embodiment of the present invention relates to a compound of formula (I), wherein A is A4 which is selected from the group consisting of

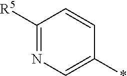

and substituents R¹, R², R³, R⁴ and R⁵ are defined as in any of the preceding embodiments.

Another embodiment of the present invention relates to a compound of formula (I), wherein A is A5 which is selected from the group consisting of

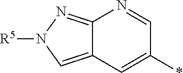

and substituents R¹, R², R³, R⁴ and R⁵ are defined as in any of the preceding embodiments.

Another embodiment of the present invention relates to a compound of formula (I), wherein R¹ is selected from the group Rib, consisting of H and F;
and substituents R², R³, R⁴ and R⁵ are defined as in any of the preceding embodiments.

Another embodiment of the present invention relates to a compound of formula (I), wherein R² is selected from the group R2b, consisting of H, C₁₋₆-alkyl and C₃₋₆-cycloalkyl;
and substituents R¹, R³, R⁴ and R⁵ are defined as in any of the preceding embodiments.

Another embodiment of the present invention relates to a compound of formula (I), wherein R² is selected from the group R2c, consisting of H, H₃C, H₃CH₂C and cyclopropyl;
and substituents R¹, R³, R⁴ and R⁵ are defined as in any of the preceding embodiments.

Another embodiment of the present invention relates to a compound of formula (I), wherein R² is selected from the group R2d, consisting of H₃C, H₃CH₂C and cyclopropyl;
and substituents R¹, R³, R⁴ and R⁵ are defined as in any of the preceding embodiments.

Another embodiment of the present invention relates to a compound of formula (I), wherein R³ is selected from the group R3b, consisting of H, C₁₋₆-alkyl and C₃₋₆-cycloalkyl;
and substituents R¹, R², R⁴ and R⁵ are defined as in any of the preceding embodiments.

Another embodiment of the present invention relates to a compound of formula (I), wherein R³ is selected from the group R3c, consisting of H, H₃C, H₃CH₂C and cyclopropyl;
and substituents R¹, R², R⁴ and R⁵ are defined as in any of the preceding embodiments.

Another embodiment of the present invention relates to a compound of formula (I), wherein R³ is selected from the group R3d, consisting of H₃C, H₃CH₂C and cyclopropyl; and substituents R¹, R², R⁴ and R⁵ are defined as in any of the preceding embodiments.

Another embodiment E2+3a of the present invention relates to a compound of formula (I), wherein R² is selected from the group R2a, consisting of H, C₁₋₆-alkyl, F₁₋₉-fluoro-C₁₋₆-alkyl and C₃₋₆-cycloalkyl;
R³ is selected from the group R3a, consisting of H, C₁₋₆-alkyl, F₁₋₉-fluoro-C₁₋₆-alkyl and C₃₋₆-cycloalkyl;
with the proviso that one of R² or R³ is H;
or R² and R³ together with the carbon atom to which they are attached, form a cyclopropyl ring or a cyclobutyl ring; wherein said cyclopropyl or cyclobutyl ring is optionally substituted with one or two F;
and substituents R¹, R⁴ and R⁵ are defined as in any of the preceding embodiments.

Another embodiment E2+3b of the present invention relates to a compound of formula (I),
wherein R² is selected from the group R2a, consisting of H, C₁₋₆-alkyl, F₁₋₉-fluoro-C₁₋₆-alkyl and C₃₋₆-cycloalkyl;
R³ is selected from the group R3a, consisting of H, C₁₋₆-alkyl, F₁₋₉-fluoro-C₁₋₆-alkyl and C₃₋₆-cycloalkyl;
or R² and R³ together with the carbon atom to which they are attached, form a cyclopropyl ring; and substituents R¹, R⁴ and R⁵ are defined as in any of the preceding embodiments.

Another embodiment E2+3c of the present invention relates to a compound of formula (I), wherein R² is selected from the group R2a, consisting of H, C₁₋₆-alkyl, F₁₋₉-fluoro-C₁₋₆-alkyl and C₃₋₆-cycloalkyl;
R³ is selected from the group R3a, consisting of H, C₁₋₆-alkyl, F₁₋₉-fluoro-C₁₋₆-alkyl and C₃₋₆-cycloalkyl;
with the proviso that one of R² or R³ is H;
or R² and R³ together with the carbon atom to which they are attached, form a cyclopropyl ring; and substituents R¹, R⁴ and R⁵ are defined as in any of the preceding embodiments.

Another embodiment E2+3d of the present invention relates to a compound of formula (I), wherein R² is H and R³ is selected from the group R3d, consisting of H₃C, H₃CH₂C and cyclopropyl;
or R² and R³ together with the carbon atom to which they are attached, form a cyclopropyl ring; and substituents R¹, R⁴ and R⁵ are defined as in any of the preceding embodiments.

Another embodiment E2+3e of the present invention relates to a compound of formula (I), wherein R³ is H and R² is selected from the group R2d, consisting of H₃C, H₃CH₂C and cyclopropyl;
or R² and R³ together with the carbon atom to which they are attached, form a cyclopropyl ring; and substituents R¹, R⁴ and R⁵ are defined as in any of the preceding embodiments.

Another embodiment of the present invention relates to a compound of formula (I), wherein R⁴ is selected from the group R4b, consisting of H and F₁₋₉-fluoro-C₁₋₄-alkyl;
and substituents R¹, R², R³ and R⁵ are defined as in any of the preceding embodiments.

Another embodiment of the present invention relates to a compound of formula (I), wherein R⁴ is selected from the group R4c, consisting of H and F₃C;
and substituents R¹, R², R³ and R⁵ are defined as in any of the preceding embodiments.

Another embodiment of the present invention relates to a compound of formula (I), wherein R⁵ is selected from the group R5b, consisting of H, halo and C₁₋₆-alkyl;
and substituents R¹, R², R³ and R⁴ are defined as in any of the preceding embodiments.

Another embodiment of the present invention relates to a compound of formula (I), wherein R⁵ is selected from the group R5c, consisting of H, F and (H₃C)₃C;
and substituents R¹, R², R³ and R⁴ are defined as in any of the preceding embodiments.

Another embodiment of the present invention relates to a compound of formula (I), wherein the stereochemistry at the 4-position of the piperidine ring is 4R when R¹ is H or 4S when R¹ is F
and substituents R², R³ and R⁴ are defined as in any of the preceding embodiments.

Another embodiment of the present invention relates to a compound of formula (I) above, having formula (I-a)

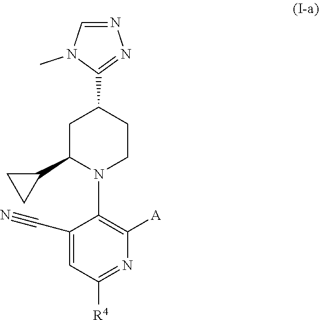

wherein substituents A, R⁴ and R⁵ are defined as in any of the preceding embodiments.

Another embodiment of the present invention relates to a compound of formula (I) above, having formula (I-b)

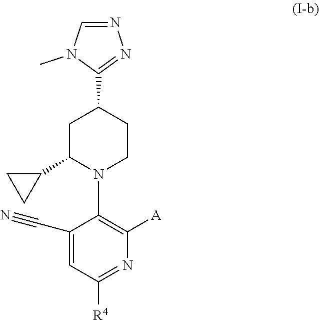

wherein substituents A, R⁴ and R⁵ are defined as in any of the preceding embodiments.

Another embodiment of the present invention relates to a compound of formula (I) above, having formula (I-c)

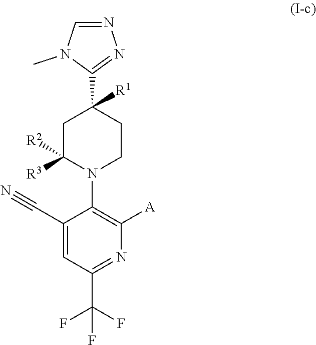

wherein substituents A, R¹, R², R³ and R⁵ are defined as in any of the preceding embodiments.

Another embodiment of the present invention relates to a compound of formula (I) above, having formula (I-d)

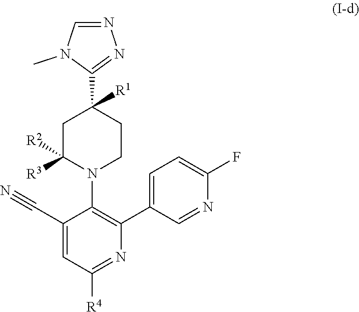

wherein substituents R¹, R², R³ and R⁴ are defined as in any of the preceding embodiments.

Another embodiment of the present invention relates to a compound of formula (I) above, having formula (I-e)

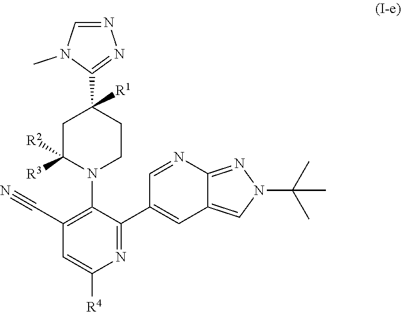

wherein substituents R¹, R², R³ and R⁴ are defined as in any of the preceding embodiments.

Particularly preferred is the compound according to formula (I) selected from the group consisting of

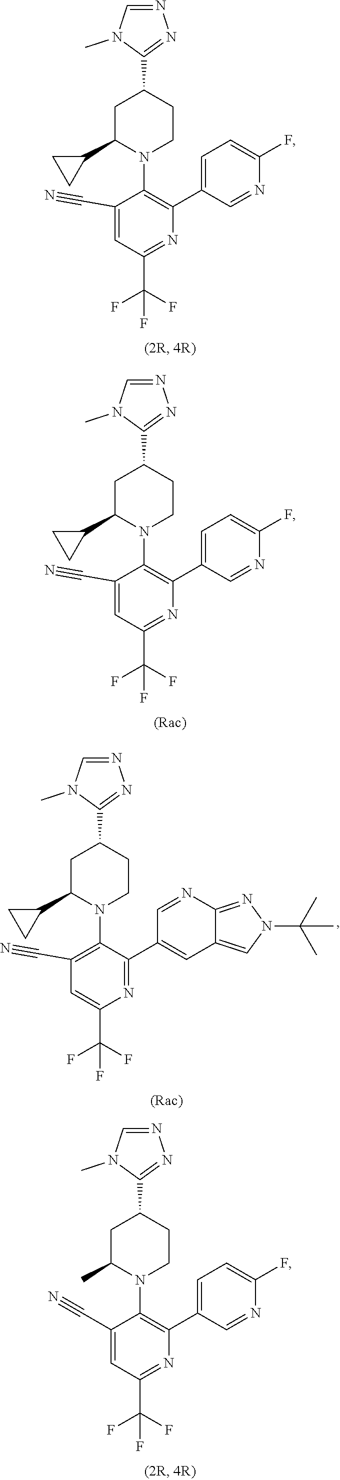 [SMILES: Cn1cnnc1[C@@H]1CCN(c2c(C#N)cc(C(F)(F)F)nc2-c2ccc(F)nc2)[C@@H](C2CC2)C1 ; Cn1cnnc1[C@@H]1CCN(c2c(C#N)cc(C(F)(F)F)nc2-c2ccc(F)nc2)[C@@H](C2CC2)C1 ; Cn1cnnc1[C@@H]1CCN(c2c(C#N)cc(C(F)(F)F)nc2-c2cnc3nn(C(C)(C)C)cc3c2)[C@@H](C2CC2)C1 ; C[C@H]1C[C@H](c2nncn2C)CCN1c1c(C#N)cc(C(F)(F)F)nc1-c1ccc(F)nc1]

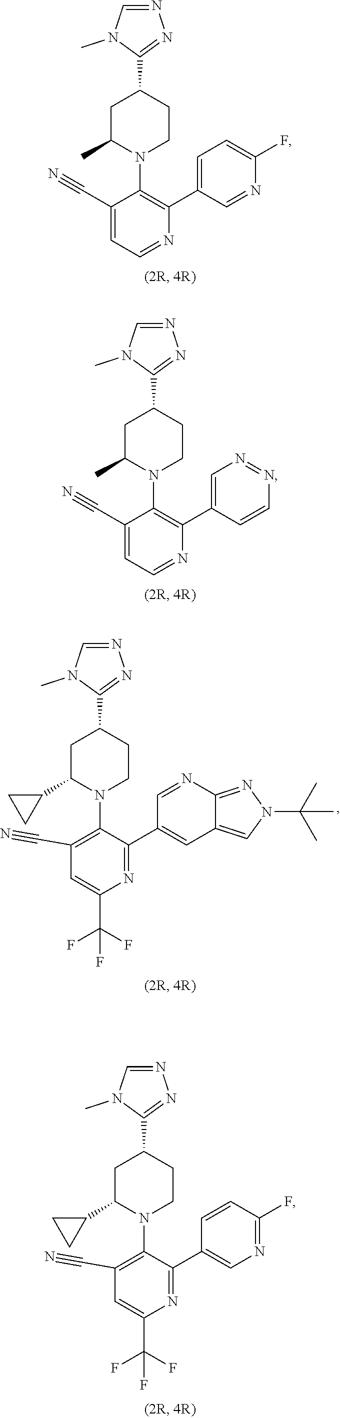 [SMILES: C[C@H]1C[C@H](c2nncn2C)CCN1c1c(C#N)ccnc1-c1ccc(F)nc1 ; C[C@H]1C[C@H](c2nncn2C)CCN1c1c(C#N)ccnc1-c1ccnnc1 ; Cn1cnnc1[C@@H]1CCN(c2c(C#N)cc(C(F)(F)F)nc2-c2cnc3nn(C(C)(C)C)cc3c2)[C@H](C2CC2)C1 ; Cn1cnnc1[C@@H]1CCN(c2c(C#N)cc(C(F)(F)F)nc2-c2ccc(F)nc2)[C@H](C2CC2)C1]

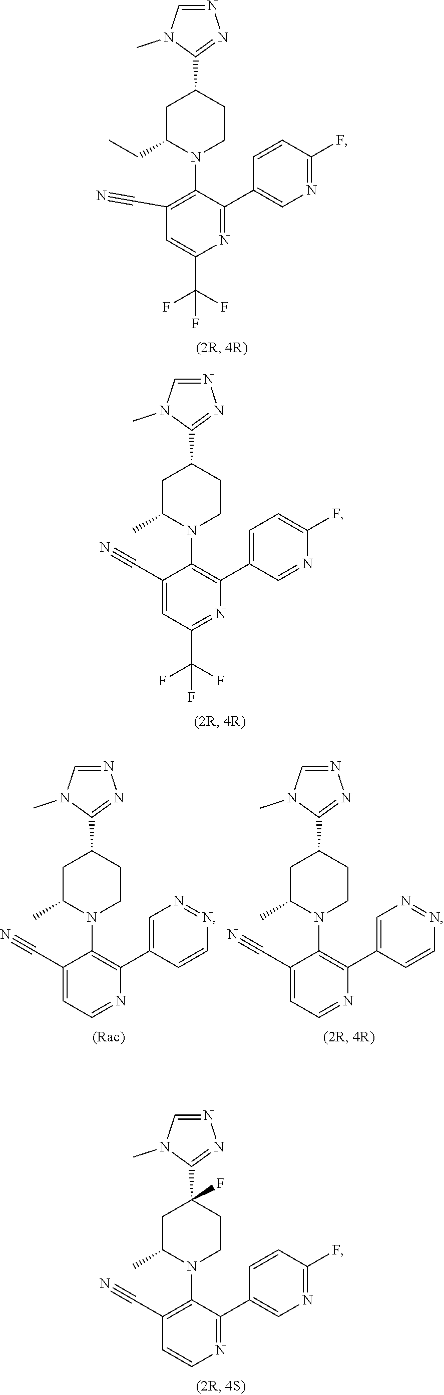

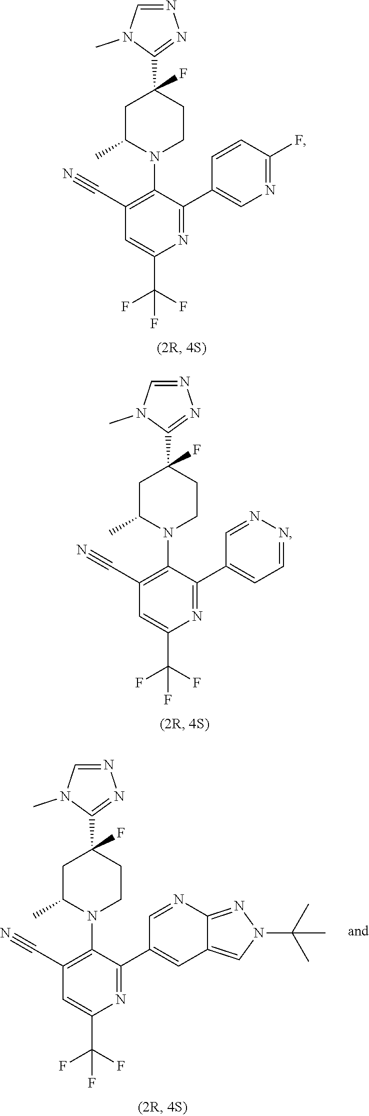 [SMILES: C[C@@H]1C[C@](F)(c2nncn2C)CCN1c1c(C#N)cc(C(F)(F)F)nc1-c1ccc(F)nc1 ; C[C@@H]1C[C@](F)(c2nncn2C)CCN1c1c(C#N)cc(C(F)(F)F)nc1-c1ccnnc1 ; C[C@@H]1C[C@](F)(c2nncn2C)CCN1c1c(C#N)cc(C(F)(F)F)nc1-c1cnc2nn(C(C)(C)C)cc2c1]

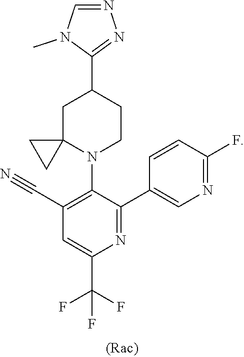 [SMILES: Cn1cnnc1C1CCN(c2c(C#N)cc(C(F)(F)F)nc2-c2ccc(F)nc2)C2(CC2)C1]

Particularly preferred is the compound according to formula (I) selected from the group consisting of example 1, example 2, example 3, example 7, example 8, example 13 and example 16 as described hereinafter in EXAMPLES.

The present invention provides novel piperidinylpyridinylcarbonitrile derivatives of formula (I) that are surprisingly potent QPCT/L inhibitors.

Another aspect of the invention refers to compounds according to formula (I) as surprisingly having potent inhibition of QPCT/L in cells relevant for, but not limited to, lung diseases or cancer.

Another aspect of the invention refers to compounds according to formula (I) as surprisingly cellular potent QPCT/L inhibitors having appropriate membrane permeability and low in vitro efflux.

Another aspect of the invention refers to pharmaceutical compositions, containing at least one compound according to formula (I) optionally together with one or more inert carriers and/or diluents.

A further aspect of the present invention refers to compounds according to formula (I), for the use in the prevention and/or treatment of disorders associated with QPCT/L inhibition.

Another aspect of the invention refers to processes of manufacture of the compounds of the present invention.

Further aspects of the present invention will become apparent to the skilled artisan directly from the foregoing and following description and the examples.

### USED TERMS AND DEFINITIONS

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

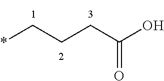 [SMILES: *CCCC(=O)O]

is wherein the carboxy group is attached to the third carbon atom of the propyl group. The terms “1-methylpropyl-”, “2,2-dimethylpropyl-” or “cyclopropylmethyl-” group represent the following groups:

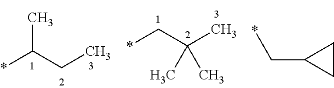

The asterisk may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates. Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof.

Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine.

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “mono-heteroaryl ring” means a monocyclic aromatic ring system, containing one or more heteroatoms selected from N, O or S, consisting of 5 to 6 ring atoms.

The term “mono-heteroaryl ring” is intended to include all the possible isomeric forms. Thus, the term “mono-heteroaryl ring” includes the following exemplary structures (not depicted as radicals as each form is optionally attached through a covalent bond to any atom so long as appropriate valences are maintained):

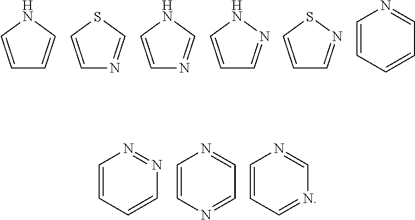

The term “fused bicyclic-heteroaryl ring” means a bicyclic aromatic ring system, containing one or more heteroatoms selected from N, O or S, consisting of 9 to 10 ring atoms. The term “fused bicyclic-heteroaryl ring” is intended to include all the possible isomeric forms. Thus, the term “bicyclic-heteroaryl ring” includes the following exemplary structures (not depicted as radicals as each form is optionally attached through a covalent bond to any atom so long as appropriate valences are maintained):

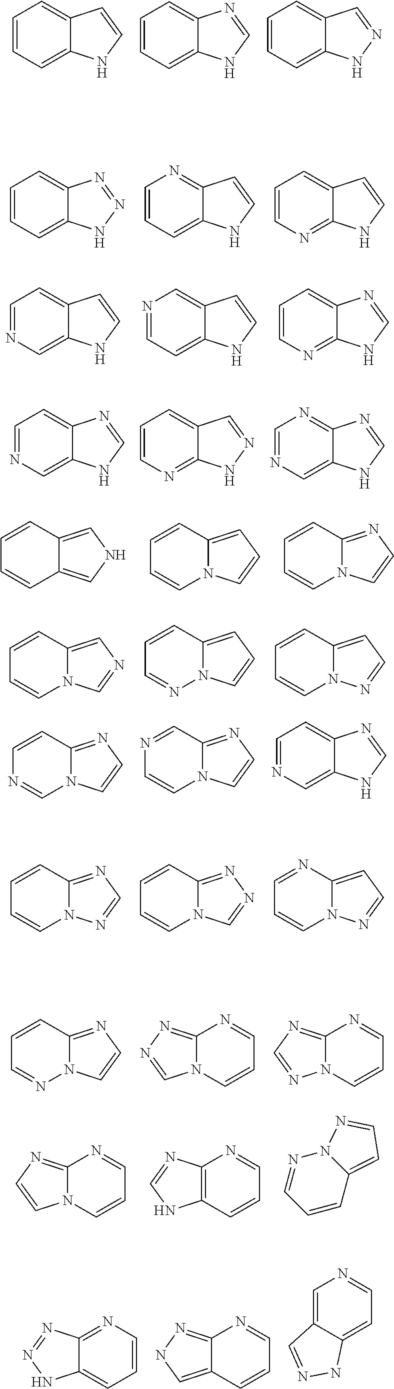

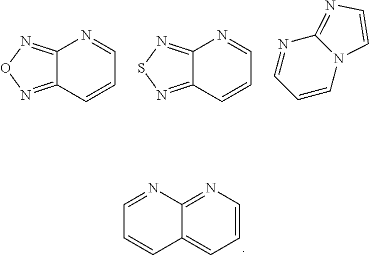

The term pyridyl refers to the radical of the following ring:

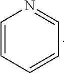

The term pyridazinyl refers to the radical of the following ring:

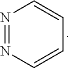

The term 2H-pyrazolo[3,4-b]pyridyl refers to the radical of the following ring:

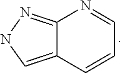

Many of the terms given above may be used repeatedly in the definition of a formula or group and in each case have one of the meanings given above, independently of one another.

### Biological Assays

### Evaluation of Inhibitory Activity on QPCT and QPCTL

### Assay A: Biochemical QPCT and QPCTL Activity Assay

The activity of the compounds of the invention may be demonstrated using the following biochemical enzyme activity assay:

QPCT or QPCTL dependent conversion of N-terminal glutamine to pyroglutamate of CD47 was monitored via MALDI-TOF MS. Test compounds were dissolved in 100% DMSO and serially diluted into clear 1,536-well microtiter plates. Enzymatic reactions were set up in assay buffer containing 20 mM Tris pH 7.5, 0.1 mM TCEP, 0.01% BSA, and 0.001% Tween20. 2.5 μL of 2× concentrated QPCTL (in-house) or QPCT (Origine #TP700028) enzyme in assay buffer (0.5 nM final concentration, columns 1-23) or plain assay buffer (columns 24) were added to each well. The plates were incubated for 10 min in a humidified incubator at 24° C. Subsequently, 2.5 μL of CD47 peptide substrate surrogate (¹⁹QLLFNKTKSVEFTFC³³) was added to each well (final concentration: 10 μM for QPCTL/20 μM for QPCT). The plates were mixed for 30 sec at 1,000 rpm and subsequently incubated for 40 min in a humidified incubator at 24° C. After incubation, the enzymatic reaction was stopped by adding 1 μL containing stable isotope labeled internal standard peptide ¹⁹[Pyr]LLFN(K)TKSVEFTFC³³ (final concentration 4.0 μM) as well as SEN177 (final concentration 10 μM). The plates were sealed with an adhesive foil, mixed for 30 s at 1,000 rpm and stored at room temperature until preparation of the MALDI target plates. MALDI target plates were prepared as described previously.1 Mass spectra were acquired with a rapifleX MALDI-TOF/TOF instrument tracking the signals of the product (¹⁹[Pyr]LLFNKTKSVEFTFC³³, m/z 1,787.9037) as well as internal standard (¹⁹[Pyr]LLFN(K)TKSVEFTFC³³, m/z 1,795.9179) peptide. QPCT or QPCTL activity was monitored by calculating the ratio between product and internal standard signals followed by normalization to high (100% activity) and low (0% activity) controls. Determination of compound potencies was obtained by fitting the dose-response data to a four-parameter logistical equation.

TABLE 2
Biological data for compounds of the invention as obtained in Assay A.

| | Inhibition of | Inhibition of |
|---|---|---|
| | QPCTL: | QPCT: |
| Example | IC₅₀ [nM] | IC₅₀ [nM] |
| 1 | 1 | 2 |
| 2 | 2 | 2 |
| 3 | 1 | 1 |
| 4 | 6 | 74 |
| 5 | 1 | 2 |
| 6 | 2 | 3 |
| 7 | 1 | 1 |
| 8 | 1 | 6 |
| 9 | 18 | 50 |
| 10 | 10 | 23 |
| 11 | 3 | 27 |
| 12 | 2 | 22 |
| 13 | 6 | 14 |
| 14 | 16 | 24 |
| 15 | 14 | 24 |
| 16 | 7 | 5 |
| 17 | 3 | 13 |

TABLE 3
Biological data for prior art compounds as obtained in Assay A.

| | | Inhibition of | Inhibition of |
|---|---|---|---|
| Prior art | | QPCTL: | QPCT: |
| Compound | Reference | IC₅₀ [nM] | IC₅₀ [nM] |
| SEN177 | J-S, NCB 2015; P, | 17 | 79 |
| | JBIC 2018 | | |
| SEN180 | J-S, NCB 2015 | 39 | 176 |
| 1094 | WO 2018/178384 | 111 | 1707 |
| 1095 | WO 2018/178384 | 13 | 260 |
| 3 | WO 2022/086920 | 52 | 482 |
| alternative 3 | WO 2022/086920 | 837 | 4691 |
| 6 | WO 2022/086920 | 1 | 1 |
| 21 | CN 114874186 | 5493 | 1398 |
| 23 | CN 114874186 | 4772 | 1845 |

### Assay B: SIRPα Signalling Assay (Using Either Raji or A549 Cells)

The activity of the compounds of the invention may be demonstrated using the following SIRPα signalling assay that measures SIRPα engagement induced by CD47 presented via cell-cell interaction. Two cell types are independently used: the Raji cell line (lymphoblast-like human cell line derived from B lymphocytes from a Burkitt's lymphoma patient in 1963) and A549 cells (adenocarcinomic human alveolar basal epithelial cells).

Test compounds were dissolved in 100% DMSO and serially diluted into a white 384-well microtiter cell culture plate (PerkinElmer #60076780 in case of Raji assay; PDL-coated plates Greiner #781945 in case of A549 assay). 5000 Raji cells (ATCC #CC86) or 5000 A549 cells (ATCC #CCL-185) in Assay Complete Cell Plating reagent 30 (DiscoverX 93-0563R30B) were added per well. The assay plate was incubated for 48 h at 37° C., 95% humidity and 5% CO₂. 15000 reporter cells (Jurkat PathHunter SIRPαV1, DiscoverX #93-1135C19) were added to each well, and the plate was incubated for 5 h at 37° C., 95% humidity and 5% CO₂. Bioassay reagent 1 of the PathHunter Bioassay detection kit (DiscoverX 93-0001) was added to each well of the plate using a multichannel pipette followed by a 15 min incubation at room temperature. Afterwards bioassay reagent 2 was added followed by 60 min incubation at room temperature (incubation in the dark). The analysis of the data was performed using the luminescence signal generated by beta-galactosidase in the PathHunter reporter cell line. The luminescence measurement was done using a Pherastar Multi-Mode Reader. Dose-response curves & IC₅₀ data were calculated with 4-parameter sigmoidal dose response formula.

TABLE 4
Biological data for compounds of the invention as obtained in Assay B.

| | Inhibition of | Inhibition of |
|---|---|---|
| | SIRPα signalling | SIRPα signalling |
| | induced by Raji | induced by A549 |
| | cells: | cells: |
| Example | IC₅₀ [nM] | IC₅₀ [nM] |
| 1 | 27 | 27 |
| 2 | 66 | 41 |
| 3 | 11 | 11 |
| 4 | 925 | |
| 5 | 48 | 57 |
| 6 | 540 | 551 |
| 7 | 9 | 12 |
| 8 | 47 | 47 |
| 9 | 325 | 109 |
| 10 | 1097 | 111 |
| 11 | 89 | |
| 12 | 137 | 151 |
| 13 | 301 | 236 |
| 14 | 621 | 232 |
| 15 | 837 | 486 |
| 16 | 124 | 74 |
| 17 | 246 | 86 |

TABLE 5
Biological data for prior art compounds as obtained in Assay B.

| | | Inhibition of | Inhibition of |
|---|---|---|---|
| | | SIRPα signalling | SIRPα signalling |
| | | induced by Raji | induced by A549 |
| Prior art | | cells: | cells: |
| Compound | Reference | IC₅₀ [nM] | IC₅₀ [nM] |
| SEN177 | J-S, NCB 2015; P, | 1365 | 214 |
| | JBIC 2018 | | |
| SEN180 | J-S, NCB 2015 | 2973 | 868 |
| 1094 | WO 2018/178384 | 3240 | 3059 |
| 1095 | WO 2018/178384 | 2203 | 161 |
| 3 | WO 2022/086920 | 2736 | 921 |
| alternative 3 | WO 2022/086920 | >10000 | >10000 |
| 6 | WO 2022/086920 | 8 | 12 |
| 21 | CN 114874186 | >10000 | >10000 |
| 23 | CN 114874186 | >10000 | >10000 |

### Evaluation of Permeability

### Assay C: Permeability in CACO-2 Cells

Caco-2 cells (1-2×10⁵ cells/1 cm² area) are seeded on filter inserts (Costar transwell polycarbonate or PET filters, 0.4 m pore size) and cultured (DMEM) for 10 to 25 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO₄, 1.8 mM CaCl₂, 4.17 mM NaHCO₃, 1.19 mM Na₂HPO₄×7H₂O, 0.41 mM NaH₂PO₄xH₂O, 15 mM HEPES, 20 mM glucose, 0.25% BSA, pH 7.2) to prepare the transport solutions (0.1-300 μM compound, final DMSO <=0.5%). The transport solution (TL) is applied to the apical or basolateral donor side for measuring A-B or B-A permeability (3 filter replicates), respectively. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

Efflux ratio (ER)=permeability B-A/permeability A-B

TABLE 7
Biological data for compounds of the invention as obtained in Assay C.

| | Permeability A-B | |
|---|---|---|
| Example | [10⁻⁶ cm/s] | Efflux Ratio |
| 1 | 17.0 | 4.4 |
| 2 | 13.0 | 6.1 |
| 3 | 1.1 | 39.1 |
| 4 | 15.0 | 4.6 |
| 5 | 3.6 | 5.0 |
| 6 | 0.2 | 8.4 |
| 7 | 2.0 | 23.5 |
| 8 | 22.0 | 3.3 |
| 9 | 22.0 | 3.8 |
| 10 | 7.8 | 5.8 |
| 11 | | |
| 12 | 3.7 | 6.5 |
| 13 | 22.0 | 2.4 |
| 14 | 58.0 | 1.2 |
| 15 | 3.3 | 9.4 |
| 16 | 9.2 | 7.1 |
| 17 | 5.1 | 10.4 |

TABLE 8
Biological data for prior art compounds as obtained in Assay C.

| Prior art | | Permeability A-B | |
|---|---|---|---|
| Compound | Reference | [10⁻⁶ cm/s] | Efflux Ratio |
| SEN177 | J-S, NCB 2015; P, | 11.0 | 3.3 |
| | JBIC 2018 | | |
| SEN180 | J-S, NCB 2015 | 4.2 | 7.1 |
| 1094 | WO 2018/178384 | 73 | 1.0 |
| 1095 | WO 2018/178384 | 68 | 0.5 |
| 3 | WO 2022/086920 | 93.0 | 0.8 |
| alternative 3 | WO 2022/086920 | 97.0 | 0.7 |
| 6 | WO 2022/086920 | 0.2 | 65.0 |
| 21 | CN 114874186 | 0.7 | 27.3 |
| 23 | CN 114874186 | 3.5 | 17.6 |

### Evaluation of Microsomal Clearance

### Microsomal Clearance:

The metabolic degradation of the test compound was assayed at 37° C. with pooled liver microsomes from various species. The final incubation volume of 60 μl per time point contains TRIS buffer pH 7.6 at room temperature (0.1 M), magnesium chloride (5 mM), microsomal protein (1 mg/mL for human and dog, 0.5 mg/mL for other species) and the test compound at a final concentration of 1 μM. Following a short preincubation period at 37° C., the reactions were initiated by addition of betanicotinamide adenine dinucleotide phosphate, reduced form (NADPH, 1 mM), and terminated by transferring an aliquot into solvent after different time points. After centrifugation (10000 g, 5 min), an aliquot of the supernatant was assayed by LC-MS/MS for the amount of parent compound. The half-life was determined by the slope of the semi-logarithmic plot of the concentration-time profile.

The intrinsic clearance (CL_INTRINSIC) is calculated by considering the amount of protein in the incubation:

$$CL\_INTRINSIC [ μ1 / min / mg protein ] = ( Ln 2 / ( half - life [ min ] * protein content [ mg / ml ] ) ) * 1000$$

$$CL\_INTRINSIC \_INVIVO [ ml / min / kg ] = ( CL\_INTRINSIC [ μL / min / mg protein ] \times MPPGL [ mg protein / g liver ] \times liver factor [ g / kg bodyweight ] ) / 1000$$

$$Qh [ \% ] = CL [ ml / min / kg ] / hepatic blood flow [ ml / min / kg ] )$$

$$Hepatocellularity , human : 120 \times 10 e 6 cells / g liver$$

$$Liver factor , human : 25.7 g / kg bodyweight$$

$$Blood flow , human : 21 ml / ( min \times kg )$$

### Evaluation of Hepatocyte Clearance

### Hepatocyte Clearance

The metabolic degradation of a test compound is assayed in a human hepatocyte suspension. After recovery from cryopreservation, human hepatocytes are diluted in Dulbecco's modified eagle medium (supplemented with 3.5 μg glucagon/500 mL, 2.5 mg insulin/500 mL, 3.75 mg hydrocortisone/500 mL, 5% human serum) to obtain a final cell density of 1.0×10⁶ cells/mL.

Following a 30 minutes preincubation in a cell culture incubator (37° C., 10% CO₂), test compound solution is spiked into the hepatocyte suspension, resulting in a final test compound concentration of 1 μM and a final DMSO concentration of 0.05%.

The cell suspension is incubated at 37° C. (cell culture incubator, horizontal shaker) and samples are removed from the incubation after 0, 0.5, 1, 2, 4 and 6 hours. Samples are quenched with acetonitrile (containing internal standard) and pelleted by centrifugation.

The supernatant is transferred to a 96-deepwell plate, and prepared for analysis of decline of parent compound by HPLC-MS/MS.

The percentage of remaining test compound is calculated using the peak area ratio (test compound/internal standard) of each incubation time point relative to the time point 0 peak area ratio. The log-transformed data are plotted versus incubation time, and the absolute value of the slope obtained by linear regression analysis is used to estimate in vitro half-life (T1/2).

In vitro intrinsic clearance (CLint) is calculated from in vitro T1/2 and scaled to whole liver using a hepatocellularity of 120×106 cells/g liver, a human liver per body weight of 25.7 g liver/kg as well as in vitro incubation parameters, applying the following equation:

$$CL\_INTRINSIC \_IN \_VIVO [ mL / min / kg ] = ( CL\_INTRINSIC [ μL / min / 106 cells ] \times hepatocellularity [ 106 cells / g liver ] \times liver factor [ g / kg body weight ] ) / 100$$

Hepatic in vivo blood clearance (CL) is predicted according to the well-stirred liver model considering an average liver blood flow (QH) of 20.7 mL/min/kg:

$$CL [ mL / min / kg ] = CL\_INTRINSIC \_IN VIVO [ mL / min / kg ] \times hepatic blood flow [ mL / min / kg ] / ( CL\_INTRINSIC \_IN VIVO [ mL / min / kg ] + hepatic blood flow [ mL / min / kg ] )$$

Results are expressed as percentage of hepatic blood flow:

$$QH [ \% ] = CL [ mL / min / kg ] / hepatic blood flow [ mL / min / kg ] )$$

### Evaluation of Plasma Protein Binding

Equilibrium dialysis technique is used to determine the approximate in vitro fractional binding of test compounds to plasma proteins applying Dianorm Teflon dialysis cells (micro 0.2). Each dialysis cell consists of a donor and an acceptor chamber, separated by an ultrathin semipermeable membrane with a 5 kDa molecular weight cutoff. Stock solutions for each test compound are prepared in DMSO at 1 mM and serially diluted to obtain a final test concentration of 1 μM. The subsequent dialysis solutions are prepared in plasma (supplemented with NaEDTA as anticoagulant), and aliquots of 200 μl test compound dialysis solution in plasma are dispensed into the donor (plasma) chambers. Aliquots of 200 μl dialysis buffer (100 mM potassium phosphate, pH 7.4, supplemented with up to 4.7% Dextran) are dispensed into the buffer (acceptor) chamber. Incubation is carried out for 2 hours under rotation at 37° C. for establishing equilibrium.

At the end of the dialysis period, aliquots obtained from donor and acceptor chambers, respectively, are transferred into reaction tubes and processed for HPLC-MS/MS analysis. Analyte concentrations are quantified in aliquots of samples by HPLC-MS/MS against calibration curves.

Percent bound is calculated using the formula:

$$\% bound = ( plasma concentration - buffer concentration / plasma concentration ) \times 100$$

### Evaluation of Solubility

Saturated solutions are prepared in well plates (format depends on robot) by adding an appropriate volume of selected aqueous media (typically in the range of 0.25-1.5 ml) into each well which contains a known quantity of solid drug substance (typically in the range 0.5-5.0 mg). The wells are shaken or stirred for a predefined time period (typically in a range of 2-24 h) and then filtered using appropriate filter membranes (typically PTFE-filters with 0.45 μm pore size). Filter absorption is avoided by discarding the first few drops of filtrate. The amount of dissolved drug substance is determined by UV spectroscopy. In addition, the pH of the aqueous saturated solution is measured using a glass-electrode pH meter.

### Evaluation of Metabolism in Human Hepatocytes In Vitro

The metabolic pathway of a test compound is investigated using primary human hepatocytes in suspension. After recovery from cryopreservation, human hepatocytes are incubated in Dulbecco's modified eagle medium containing 5% human serum and supplemented with 3.5 μg glucagon/500 ml, 2.5 mg insulin/500 ml and 3.75 mg/500 ml hydrocortisone. Following a 30 min preincubation in a cell culture incubator (37° C., 10% CO₂), test compound solution is spiked into the hepatocyte suspension to obtain a final cell density of 1.0*10⁶ to 4.0*10⁶ cells/ml (depending on the metabolic turnover rate of the compound observed with primary human hepatocytes), a final test compound concentration of 10 μM, and a final DMSO concentration of 0.05%.

The cells are incubated for six hours in a cell culture incubator on a horizontal shaker, and samples are removed from the incubation after 0, 0.5, 1, 2, 4 or 6 hours, depending on the metabolic turnover rate. Samples are quenched with acetonitrile and pelleted by centrifugation. The supernatant is transferred to a 96-deepwell plate, evaporated under nitrogen and resuspended prior to bioanalysis by liquid chromatography-high resolution mass spectrometry for identification of putative metabolites.

The structures are assigned tentatively based on Fourier-Transform-MSⁿ data. Metabolites are reported as percentage of the parent in human hepatocyte incubation with a threshold of ≥4%.

### Evaluation of Pharmacokinetic Characteristics

The test compound is administered either intravenously or orally to the respective test species. Blood samples are taken at several time points post application of the test compound, anticoagulated and centrifuged.

The concentration of analytes—the administered compound and/or metabolites—are quantified in the plasma samples. PK parameters are calculated using non compartment methods. AUC and Cmax are normalized to a dose of 1 mol/kg.

### Method of Treatment

The present invention is directed to compounds of general formula (I) which are useful in the prevention and/or treatment of a disease and/or condition associated with or modulated by QPCT/L activity, including but not limited to the treatment and/or prevention of cancer, fibrotic diseases, neurodegenerative diseases, atherosclerosis, infectious diseases, chronic kidney diseases.

The compounds of general formula (I) are useful for the prevention and/or treatment of:
(1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural
parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment
such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or
sarcoidosis, granulomatosis, cystic fibrosis or mucoviscidosis, or alpha-I-antitrypsin deficiency.
(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.
(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.
(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.
(5) Neurodegenerative disorders such as amyotrophic lateral sclerosis, multiple sclerosis, Parkinson's disease, Alzheimer's disease, Huntington's disease, multiple system atrophy, or prion diseases.

Accordingly, the present invention relates to a compound of general formula (I) or a pharmaceutically acceptable salt thereof for use as a medicament.

Furthermore, the present invention relates to the use of a compound of general formula (I) for the treatment and/or prevention of a disease and/or condition associated with or modulated by QPCT/L activity.

Furthermore, the present invention relates to the use of a compound of general formula (I) or a pharmaceutically acceptable salt thereof or a pharmaceutical composition thereof for the treatment and/or prevention of cancer, fibrotic diseases, neurodegenerative diseases, atherosclerosis, infectious diseases, chronic kidney diseases.

Furthermore, the present invention relates to the use of a compound of general formula (I) or a pharmaceutically acceptable salt thereof or a pharmaceutical composition thereof for the treatment and/or prevention of: (1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.

(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.

(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.

(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.

(5) Neurodegenerative disorders such as amyotrophic lateral sclerosis, multiple sclerosis, Parkinson's disease, Alzheimer's disease, Huntington's disease, multiple system atrophy, or prion diseases.

In a further aspect the present invention relates to a compound of general formula (I) or a pharmaceutically acceptable salt thereof or a pharmaceutical composition thereof for use in the treatment and/or prevention of above-mentioned diseases and conditions.

In a further aspect the present invention relates to the use of a compound of general formula (I) or a pharmaceutically acceptable salt thereof or a pharmaceutical composition thereof for the preparation of a medicament for the treatment and/or prevention of above-mentioned diseases and conditions.

In a further aspect of the present invention the present invention relates to methods for the treatment or prevention of above-mentioned diseases and conditions, which method comprises the administration of an effective amount of a compound of general formula (I) or a pharmaceutically acceptable salt thereof or a pharmaceutical composition thereof to a human being.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with cancer, fibrotic diseases, Alzheimer's diseases, atherosclerosis, infectious diseases, chronic kidney diseases and auto-immune disease.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of chemotherapy, targeted cancer therapy, cancer immunotherapy, irradiation, antifibrotic agents, anti-tussive agents, anti-inflammatory agents, anti-atopic dermatitis, and broncho dilators.

Chemotherapy is a type of cancer therapy that uses one or more chemical anticancer drugs, such as cytostatic or cytotoxic substances, cell proliferation inhibitors, anti-angiogenic substances and the like. Examples include folic acid (Leucovorin), 5-Fluorouracil, Irinotecan, Oxaliplatin, cis-platin Azacytidine, gemcitabine, alkylation agents, antimitotic agents, taxanes and further state-of-the-art or standard-of-care compounds.

Targeted therapy is a type of cancer treatment that uses drugs to target specific genes and proteins that help cancer cells survive and grow. Targeted therapy includes agents such as inhibitors of growth factors (e.g. platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor), tyrosine-kinases, KRAS, BRAF, BCR-ABL, mTOR, cyclin-dependent kinases, or MDM2.

Cancer immunotherapy is a type of therapy that uses substances to stimulate or suppress the immune system to help the body fight cancer. Cancer immunotherapy includes a therapeutic antibody, such as: anti-Her2 antibody, an anti-EGFR antibody, and an anti-PDGFR antibody; an anti-GD2 (Ganglioside G2) antibody. Examples include Dinutuximab, Olaratumab, Trastuzumab, Pertuzumab, Ertumaxomab, Cetuximab, Necitumumab, Nimotuzumab, Panitumumab, or rituximab. Cancer immunotherapy also includes a therapeutic antibody which is a checkpoint inhibitor, such as an anti PD1, anti PD-L1 antibody or CTLA4 inhibitor. Examples include Atezolizumab, Avelumab, and Durvalumab, Ipilimumab, nivolumab, or pembrolizumab. Cancer immunotherapy also includes agents which target (inhibit) the CD47-SIRPα signaling axis, such as agents which bind to CD47 or SIRPα. Non-limiting examples include antibodies such as anti-CD47 antibodies and anti-SIRPα antibodies, and recombinant Fc-fusion proteins such as CD47-Fc and SIRPα-Fc. Cancer immunotherapy also includes STING-targeting agent, or T cell engagers, such as blinatumomab.

Antifibrotic agents are for example nintedanib, pirfenidone, phosphodiesterase-IV (PDE4) inhibitors such as roflumilast or specific PDE4b inhibitors like BI 1015550, autotaxin inhibitors such as GLPG-1690 or BBT-877; connective tissue growth factor (CTGF) blocking antibodies such as Pamrevlumab; B-cell activating factor receptor (BAFF-R) blocking antibodies such as Lanalumab, alpha-V/beta-6 blocking inhibitors such as BG-00011/STX-100, recombinant pentraxin-2 (PTX-2) such as PRM-151; c-Jun-N-terminal kinase (JNK) inhibitors such as CC-90001; galectin-3 inhibitors such as TD-139; G-protein coupled receptor 84 (GPR84) inhibitors; G-protein coupled receptor 84/G-protein coupled receptor 40 dual inhibitors such asPBI-4050, Rho Associated Coiled-Coil Containing Protein Kinase 2 (ROCK2) inhibitors such as KD-025, heat shock protein 47 (HSP47) small interfering RNA such as BMS-986263/ND-L02-s0201; Wnt pathway inhibitor such as SM-04646; LD4/PDE3/4 inhibitors such as Tipelukast; recombinant immuno-modulatory domains of histidyl tRNA synthetase(HARS) such as ATYR-1923, prostaglandin synthase inhibitors such as ZL-2102/SAR-191801; 15-hydroxy-eicosapentaenoic acid (15-HEPE e.g. DS-102); Lysyl Oxidase Like 2 (LOXL2) inhibitors such as PAT-1251, PXS-5382/PXS-5338; phosphoinositide 3-kinases (PI3K)/mammalian target of rapamycin (mTOR) dual inhibitors such as HEC-68498; calpain inhibitors such as BLD-2660; mitogen-activated protein kinase kinase kinase (MAP3K19) inhibitors such as MG-S-2525; chitinase inhibitors such as OATD-01, mitogen-activated protein kinase-activated protein kinase 2 (MAPKAPK2) inhibitors such as MMI-0100; transforming growth factor beta I (TGF-beta I) small interfering RNA such as TRKZSO/BNC-1021; or lysophosphatidic acid receptor antagonists such as BMS986278. The dosage for the combination partners mentioned above is usually ⅕ of the lowest dose normally recommended up to 1/1 of the normally recommended dose.

Therefore, in another aspect, this invention relates to the use of a compound according to the invention in combination with one or more additional therapeutic agents described hereinbefore and hereinafter for the treatment of diseases or conditions which may be affected or which are mediated by QPCT/L, in particular diseases or conditions as described hereinbefore and hereinafter.

In a further aspect this invention relates to a method for treating a disease or condition which can be influenced by the inhibition of QPCT/L in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of QPCT/L in a patient in need thereof.

In yet another aspect the present invention relates to a method for the treatment of a disease or condition mediated by QPCT/L activity in a patient that includes the step of administering to the patient, preferably a human, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times. The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### PREPARATION

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Any functional groups in the starting materials or intermediates may be protected using conventional protecting groups. These protecting groups may be cleaved again at a suitable stage within the reaction sequence using methods familiar to the one skilled in the art.

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis. Abbreviations are as defined in the Examples section.

Examples 1-19 may be prepared as shown in Scheme I below.

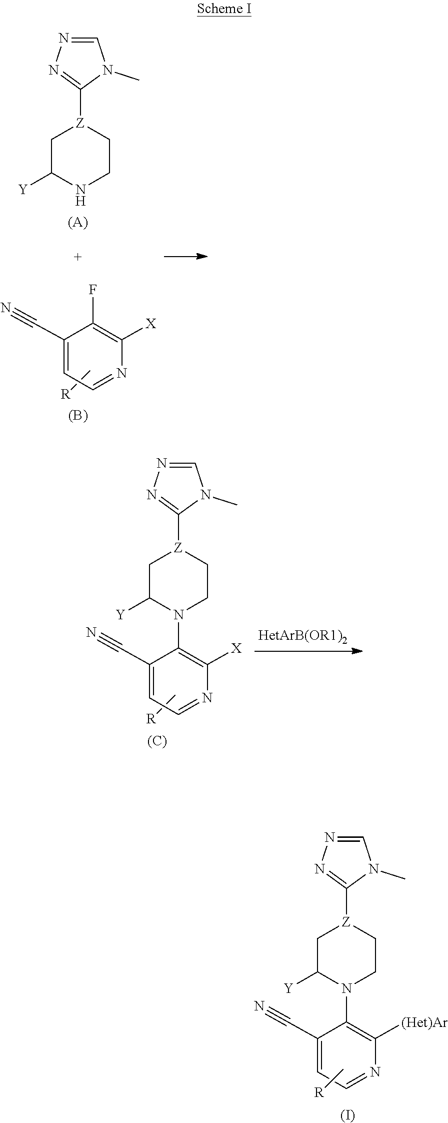

In scheme I, N-Methyl triazolyl piperidine (Z═C—H, C—F) (A) undergo a nucleophilic aromatic substitution with heteroaryl fluoride (X═Cl, Br) (B). The reaction can typically be run at ambient temperature or at elevated temperature (up to 110° C.) in the presence of a base (e.g. diisopropylethylamine). The intermediate (C) is then subjected to a Suzuki-cross coupling with a hetero-aryl boronic acid derivative in the presence of a suitable catalyst (e.g. Pd(dppf)Cl₂) and a suitable base at elevated temperature (e.g. 100° C.) to afford compounds of general formula (I).

Intermediates I may be prepared as shown in Scheme II below:

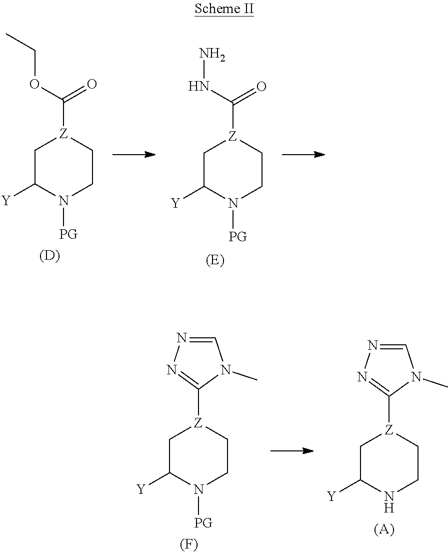

Compounds of formula (A) with Z═C—H or C—F and Y=methyl, ethyl, cyclopropyl can be prepared from the corresponding piperidinyl esters (D) equipped with a suitable protecting group (PG, e.g. Benzyl) by treatment with a suitable hydrazine source (e.g. N₂H₄*H₂O) at elevated temperature (e.g. 50° C.). The obtained hydrazide (E) is then activated with DMF/DMA at elevated temperature (e.g. 50° C.) and subsequently treated with methyl amine at elevated temperature (e.g. 90° C.) to yield the triazole derivative (F).

Compounds of formula (A) can be obtained by cleaving the protecting group under suitable conditions (e.g. H₂, Pd/C 10%, EtOH).

Intermediates II may be prepared as shown in Scheme III below:

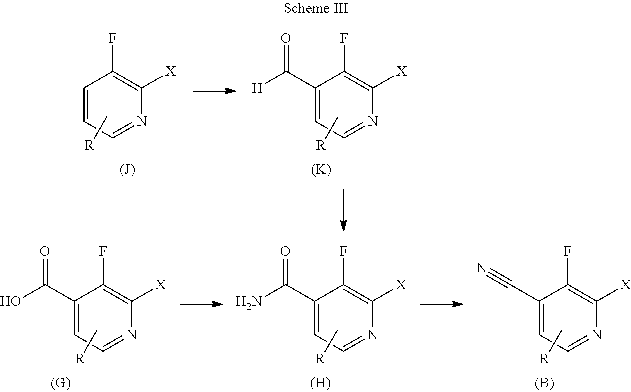

In case of R═H, intermediates of formula (B) can be prepared from the corresponding carboxylic acids (G). The carboxylic acid moiety is transformed into the corresponding amide (H) using a suitable combination of reagents, e.g. 1,1′-carbonyldiimidazole and ammonia at ambient temperature. Compounds of formula (B) are subsequently obtained by treatment of (H) with a suitable dehydrating agent, e.g. Burgess reagent at ambient temperature. In case of R=Me or CF₃, deprotonation of pyridine (J) at low temperature (e.g. −65° C.) and quenching with DMF yields the corresponding aldehydes (K). In case of R═CF₃, aldehyde (K) can be transformed into the amide (H) using a suitable reagent, e.g. phenyltrimethylammonium tribromide, at ambient temperature and further into the nitrile (B) as described above for R═H.

### EXAMPLES

### Preparation

The compounds according to the invention and their intermediates may be obtained using methods of synthesis which are known to the one skilled in the art and described in the literature of organic synthesis for example using methods described in “Comprehensive Organic Transformations”, 2nd Edition, Richard C. Larock, John Wiley & Sons, 2010, and “March's Advanced Organic Chemistry”, 7th Edition, Michael B. Smith, John Wiley & Sons, 2013. Preferably the compounds are obtained analogously to the methods of preparation explained more fully hereinafter, in particular as described in the experimental section. In some cases the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature for example in “Protecting Groups”, 3rd Edition, Philip J. Kocienski, Thieme, 2005, and “Protective Groups in Organic Synthesis”, 4th Edition, Peter G. M. Wuts, Theodora W. Greene, John Wiley & Sons, 2006. The terms “ambient temperature” and “room temperature” are used interchangeably and designate a temperature of about 20° C., e.g. between 19 and 24° C.

### Abbreviations

| | |
|---|---|
| ACN | acetonitrile |
| AcOH | acetic acid |
| Ag₂O | silver (I) oxide |
| Aq. | aqueous |
| BOC₂O | Di-tert-butyldicarbonat |
| brine | saturated aqueous NaCl solution |
| ° C. | degree celsius |
| CyH/CH | cyclohexane |
| CO₂ | carbon dioxide |
| conc. | concentrated |
| DCM | dichloromethane |
| DIPEA | N,N-diisopropylethylamine |
| DMF | N,N-dimethylformamide |
| DMF-DMA | N,N-dimethylformamide dimethyl acetal |
| DMSO | dimethyl sulfoxide |
| ESI-MS | Electrospray ionisation mass spectrometry |
| EtMgBr | ethylmagnesium bromide |
| EtOAc/EA | ethyl acetate |
| EtOH | ethanol |
| ex | example |
| eq | equivalent |
| FA | formic acid |
| h | hour |
| H₂ | Hydrogen |
| H₂O | water |
| H₂SO₄ | sulfuric acid |
| HCl | hydrochloric acid |
| HPLC | high performance liquid chromatography |
| Int. | intermediate |
| IPA | isopropylalcohol |
| IPAm | isopropylamine |
| IPTG | Isopropyl-β-D-1-thiogalactopyranoside |
| K₂CO₃ | potassium carbonate |
| L | liter |
| LDA | lithium diisopropylamide |
| M | Molar (mol/L) |
| Me₃O(BF₄) | trimethyloxonium tetrafluoroborate |
| MeOH | methanol |
| MES | 2-(N-morpholino)ethanesulfonic acid |
| min | minute |
| mL | milliliter |
| MTBE | Methyl-tert-butylether |
| μL | microliter |
| N₂ | nitrogen |
| Na₂CO₃ | sodium carbonate |
| NaCl | Sodium chloride |
| NFSI | N-fluorobenzenesulfonimide |
| NaHCO₃ | sodium bicarbonate |
| NH₃ | ammonia |
| NH₄Cl | ammonium chloride |
| Na₂SO₄ | sodium sulfate |
| OD600 | Optical density at a wavelength of 600 nm |
| Pd(dppf)Cl₂ | 1,1′-Bis(diphenylphosphino)ferrocene |
| | palladium(II)dichloride |
| Pd/C | Palladium on carbon |
| PE | petroleum ether |
| Prep. | preparative |
| RP | reversed phase |
| RT/rt | room temperature (about 20° C.) |
| sat. | saturated |
| scCO₂ | Supercriticial carbon dioxide |
| SFC | Supercritical Fluid Chromatography |
| SiO₂ | silica |
| TEA | triethylamine |
| TFA | trifluoroacetic acid |
| THF | tetrahydrofuran |
| Tris | Tris(hydroxymethyl)aminomethane |

### Preparation of Intermediates

### Synthesis of Intermediate I.1 and I.3

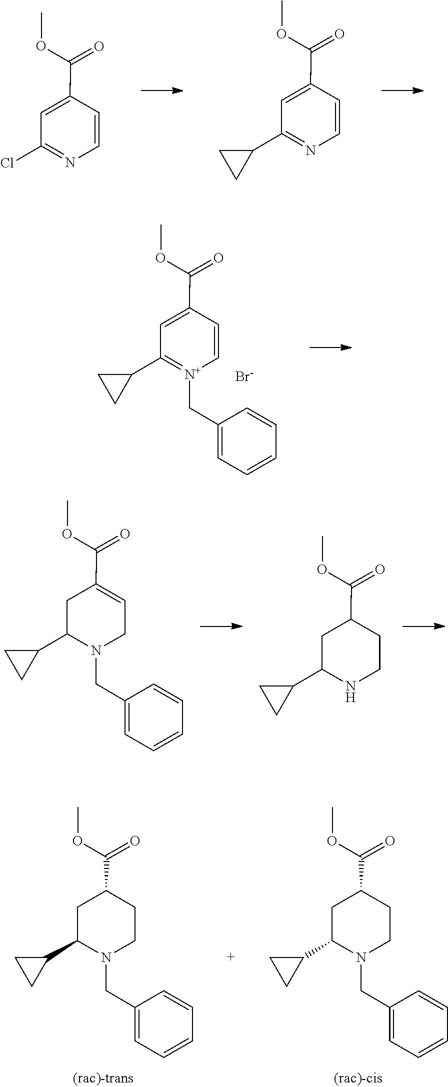

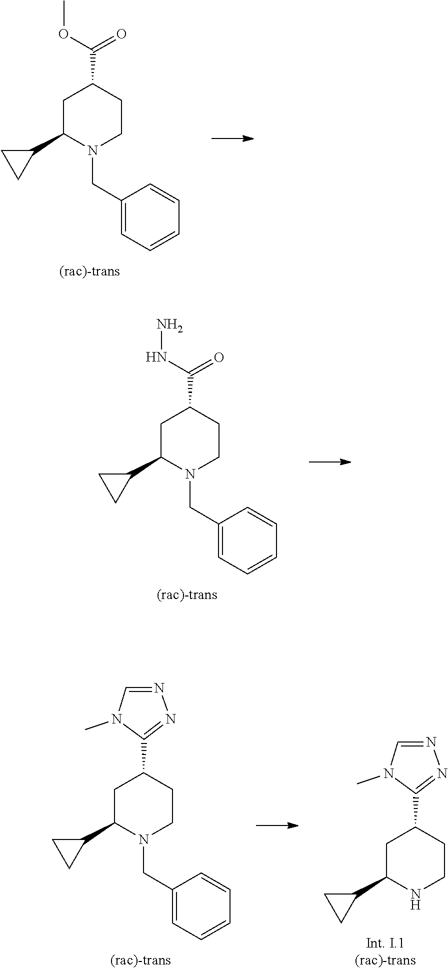 [SMILES: COC(=O)[C@@H]1CCN(Cc2ccccc2)[C@@H](C2CC2)C1 ; NNC(=O)[C@@H]1CCN(Cc2ccccc2)[C@@H](C2CC2)C1 ; Cn1cnnc1[C@@H]1CCN(Cc2ccccc2)[C@@H](C2CC2)C1 ; Cn1cnnc1[C@@H]1CCN[C@@H](C2CC2)C1]

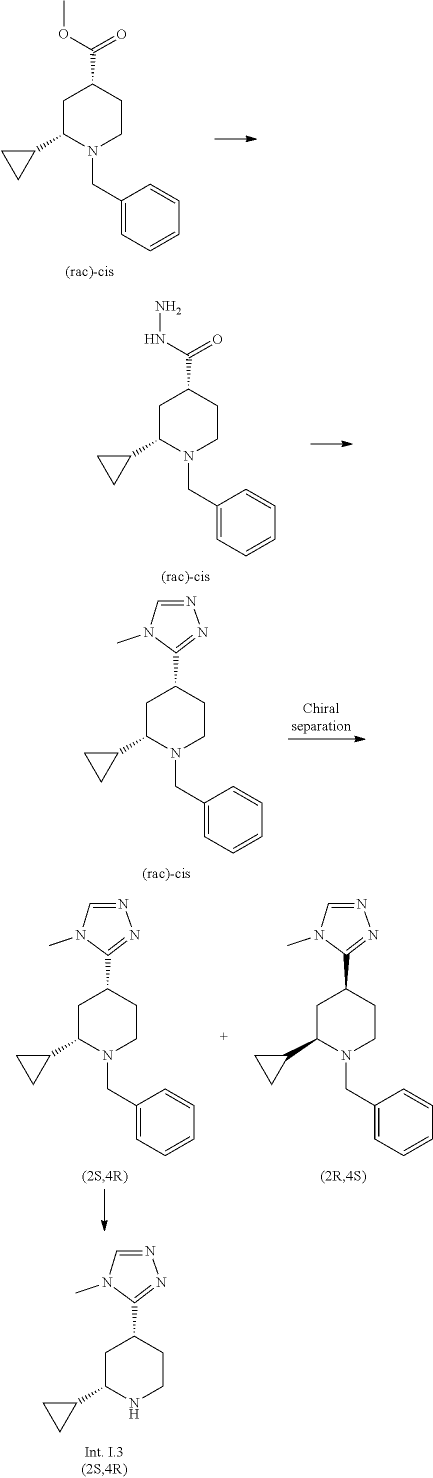

### Methyl 2-cyclopropylpyridine-4-carboxylate

Methyl 2-chloropyridine-4-carboxylate (10.0 g, 58.3 mmol), cyclopropylboronic acid (10.0 g, 117 mmol) and potassium phosphate (27.7 g, 117 mmol) are added to toluene (100 mL) and water (7.5 mL), and the resulting mixture is degassed by passing an argon stream through the mixture for 15 min. Palladium (II) acetate (1.31 g, 5.83 mmol) and tricyclohexylphosphine (1.63 g, 5.83 mmol) are added, and the mixture is degassed for additional 10 min. The mixture is then heated to 85-95° C. and stirred at this temperature for 30 h. After cooling to ambient temperature, the mixture is diluted with EtOAc. The organic layer is separated, and the aqueous layer is extracted several times with EtOAc. The combined organic layers are dried, filtered, and concentrated. The residue is purified by column chromatography (SiO₂, CyH/EtOAc gradient 100:0 to 90:10) to yield the desired compound.

| | |
|---|---|
| C₁₀H₁₁NO₂ | (M = 177.2 g/mol) |
| ESI-MS: | 178 [M + H]⁺ |
| Rt (HPLC): | 0.32 min (method A) |

### 1-Benzyl-2-cyclopropyl-4-(methoxycarbonyl)pyridin-1-ium bromide

Methyl 2-cyclopropylpyridine-4-carboxylate (10.1 g, 57.1 mmol) is dissolved in ACN (50 mL). Benzyl bromide (9.96 g, 57.1 mmol) is added, and the reaction mixture is stirred at 60° C. for 1 week (during this time two times 0.6 mL benzyl bromide are added). The reaction mixture is quenched by the addition of water and lyophilized to afford the desired compound as a crude.

| | |
|---|---|
| C₁₇H₁₈BrNO₂ | (M = 348.2 g/mol) |
| ESI-MS: | 268 [M-Br]+ |
| Rt (HPLC): | 0.41 min (method A) |

### Methyl 1-benzyl-2-cyclopropyl-1,2,3,6-tetrahydropyridine-4-carboxylate

Crude 1-benzyl-2-cyclopropyl-4-(methoxycarbonyl)pyridin-1-ium bromide (20.3 g, 58.2 mmol) is dissolved in MeOH (200 mL). At 0° C., sodium borohydride (2.50 g, 65.8 mmol) is added, and the mixture is stirred at 0° C. for 15 min. Reaction mixture is quenched by the addition of an ammonium chloride solution (50 mL) and concentrated. The aqueous residue is diluted with water and extracted with EtOAc. The organic layer is washed with brine, dried, filtered, and concentrated to afford the desired compound as a crude.

| | |
|---|---|
| C₁₇H₂₁NO₂ | (M = 271.4 g/mol) |
| ESI-MS: | 272 [M + H]⁺ |
| Rt (HPLC): | 0.38 min (method A) |

### Methyl 2-cyclopropylpiperidine-4-carboxylate

Crude methyl 1-benzyl-2-cyclopropyl-1,2,3,6-tetrahydropyridine-4-carboxylate (15.6 g, 57.5 mmol) is dissolved in MeOH (80 mL). Pd/C 10% (1.50 g) is added, and the reaction mixture is stirred under an atmosphere of H₂ (2 bar) at rt for 18 h. Reaction mixture is concentrated and re-dissolved in DCM (240 mL) and MeOH (80 mL). At −5° C. trimethylsilyl diazomethane (0.6 N, 100 mL, 60.0 mmol) is added dropwise, and the reaction mixture is stirred at rt for 2.5 h. The reaction mixture is concentrated to afford the desired crude compound, which is used without further purification.

| | |
|---|---|
| C₁₀H₁₇NO₂ | (M = 183.2 g/mol) |
| ESI-MS: | 184 [M + H]⁺ |
| Rt (HPLC): | 0.42 min (method B) |

### (rac)-Methyl trans-1-benzyl-2-cyclopropylpiperidine-4-carboxylate and (rac)-methyl cis-1-benzyl-2-cyclopropylpiperidine-4-carboxylate

Crude methyl 2-cyclopropylpiperidine-4-carboxylate (8.96 g, 48.9 mmol) is dissolved in DMF (40 mL). K₂CO₃ (20.3 g, 147 mmol) and benzyl bromide (5.96 mL, 49.1 mmol) are added, and the reaction mixture is stirred at rt over the weekend. The reaction mixture is concentrated, diluted with water, and extracted with EtOAc. The organic layer is washed with water, brine, dried, filtered, and concentrated. The residue is purified by column chromatography (SiO₂, CyH/EtOAc gradient 100:0 to 90:10) to yield the desired compounds (rac)-trans and (rac)-cis.

| | |
|---|---|
| C₁₇H₂₃NO₂ | (M = 273.4 g/mol) |
| ESI-MS: | 274 [M + H]⁺ |
| Rt (HPLC): | 1.045 min (rac)-trans and |
| | 1.044 min (rac)-cis (method C) |

### Synthesis of Intermediate I.1

### (rac)-trans-1-Benzyl-2-cyclopropylpiperidine-4-carbohydrazide

(rac)-methyl trans-1-benzyl-2-cyclopropylpiperidine-4-carboxylate (985 mg, 3.60 mmol) is dissolved in EtOH (6.00 mL). Hydrazine monohydrate (1.40 mL, 28.1 mmol) is added, and the reaction mixture is stirred at 60° C. for 40 h. Reaction mixture is concentrated, and the residue is re-dissolved in toluene and concentrated again to afford the desired compound.

| | |
|---|---|
| C₁₆H₂₃N₃O | (M = 273.4 g/mol) |
| ESI-MS: | 274 [M + H]⁺ |
| Rt (HPLC): | 0.78 min (method C) |

### (rac)-trans-1-benzyl-2-cyclopropyl-4-(4-methyl-4H-1,2,4-triazol-3-yl)piperidine

(rac)-trans-I-benzyl-2-cyclopropylpiperidine-4-carbohydrazide (1.10 g, 3.62 mmol, 90% purity) is dissolved in 1.4-dioxane (11.0 mL). N,N-dimethylformamide dimethyl acetal (1.34 mL, 10.1 mmol) is added and the reaction mixture is stirred at 50° C. for 2 h. After cooling to rt, methylamine (2 M in THF, 10.0 mL, 20.0 mmol) and glacial acetic acid (1.14 mL, 19.9 mmol) are added, and the reaction mixture is stirred at 75° C. for 30 h. The reaction mixture is concentrated, the residue is diluted with EtOAc, and washed with a K₂CO₃ solution. The organic layer is separated, dried, and concentrated. The residue is purified by preparative HPLC (XBridge C18, ACN/water gradient containing 0.1% NH₃) to yield the desired compound.

| | |
|---|---|
| C₁₈H₂₄N₄ | (M = 296.4 g/mol) |
| ESI-MS: | 297 [M + H]⁺ |
| Rt (HPLC): | 0.55 min (method B) |

### Intermediate I.1: (rac)-trans-2-cyclopropyl-4-(4-methyl-4H-1,2,4-triazol-3-yl)piperidine

(rac)-trans-I-benzyl-2-cyclopropyl-4-(4-methyl-4H-1,2,4-triazol-3-yl)piperidine (760 mg, 2.56 mmol) is dissolved in MeOH (15 mL). Pd/C 10% (150 mg) is added, and the reaction mixture is stirred under an atmosphere of H₂ (2 bar) at rt for 20 h. The reaction mixture is concentrated to afford the titled compound.

| | |
|---|---|
| C₁₁H₁₈N₄ | (M = 206.3 g/mol) |
| ESI-MS: | 207 [M + H]⁺ |
| Rt (HPLC): | 0.23 min (method B) |

### Synthesis of Intermediate I.3

### (rac)-cis-1-benzyl-2-cyclopropylpiperidine-4-carbohydrazide

(rac)-methyl cis-1-benzyl-2-cyclopropylpiperidine-4-carboxylate (7.32 g, 26.8 mmol) is dissolved in EtOH (30 mL). Hydrazine monohydrate (6.00 mL, 120.6 mmol) is added, and the reaction mixture is stirred at 60° C. for 40 h. The reaction mixture is concentrated and coevaporated with toluene (2×) and ACN (1×) to afford the desired compound.

| | |
|---|---|
| C₁₆H₂₃N₃O | (M = 273.4 g/mol) |
| ESI-MS: | 274 [M + H]⁺ |
| Rt (HPLC): | 0.79 min (method C) |

### (rac)-cis-1-benzyl-2-cyclopropyl-4-(4-methyl-4H-1,2,4-triazol-3-yl)piperidine

(rac)-cis-1-benzyl-2-cyclopropylpiperidine-4-carbohydrazide (7.31 g, 26.7 mmol) is dissolved in 1.4-dioxane (75 mL). N,N-dimethylformamide dimethyl acetal (8.88 mL, 66.9 mmol) is added and the reaction mixture is stirred at 50° C. for 2 h. After cooling to rt, methylamine (2 M in THF, 66.9 mL, 134 mmol) and glacial acetic acid (7.65 mL, 134 mmol) are added, and the reaction mixture is stirred at 70° C. for 27 h. The reaction mixture is concentrated, the residue is diluted with EtOAc, and washed with water. The organic layer is separated, dried, and concentrated. The residue is triturated with EtOAc, the precipitate is filtered and dried to afford 2 Batches of the desired compound. The aqueous layer is basified with carbonate to pH 9-10 and extracted with EtOAc. The organic layer is dried and concentrated to afford another Batch of the desired compound.

| | |
|---|---|
| C₁₈H₂₄N₄ | (M = 296.4 g/mol) |
| ESI-MS: | 297 [M + H]⁺ |
| Rt (HPLC): | 0.55 min (method B) |

### (2S,4R)-1-benzyl-2-cyclopropyl-4-(4-methyl-4H-1,2,4-triazol-3-yl)piperidine

(rac)-cis-1-benzyl-2-cyclopropyl-4-(4-methyl-4H-1,2,4-triazol-3-yl)piperidine (5.00 g, 16.9 mmol) is separated by chiral purification method E to afford the desired compound.

| | |
|---|---|
| C₁₈H₂₄N₄ | (M = 296.4 g/mol) |
| ESI-MS: | 297 [M + H]⁺ |
| Rt (HPLC): | 0.74 min (method E) |

The enantiomer ((2R,4S)-1-benzyl-2-cyclopropyl-4-(4-methyl-4H-1,2,4-triazol-3-yl)piperidine) elutes at 1.32 min (method E)

### Intermediate I.3: (2S,4R)-2-cyclopropyl-4-(4-methyl-4H-1,2,4-triazol-3-yl)piperidine

(2S,4R)-1-benzyl-2-cyclopropyl-4-(4-methyl-4H-1,2,4-triazol-3-yl)piperidine (1.89 g, 6.38 mmol) is dissolved in MeOH (60 mL). Pd/C 10% (600 mg) is added, and the reaction mixture is hydrogenated at rt for 18 h under 2 bar of H₂. The reaction mixture is concentrated to afford the titled compound.

| | |
|---|---|
| C₁₁H₁₈N₄ | (M = 206.3 g/mol) |
| ESI-MS: | 207 [M + H]⁺ |
| Rt (HPLC): | 0.27 min (method B) |

### Synthesis of Intermediate I.2

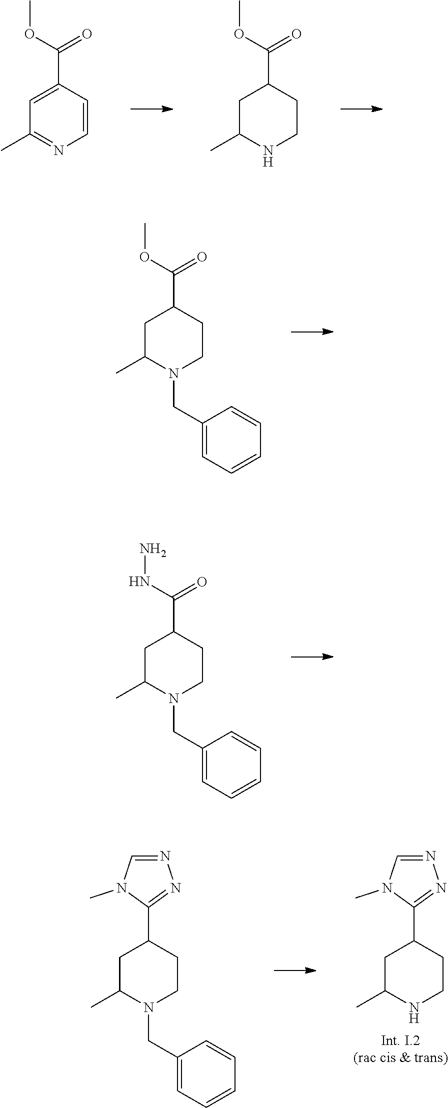 [SMILES: COC(=O)c1ccnc(C)c1 ; COC(=O)C1CCNC(C)C1 ; COC(=O)C1CCN(Cc2ccccc2)C(C)C1 ; CC1CC(C(=O)NN)CCN1Cc1ccccc1 ; CC1CC(c2nncn2C)CCN1Cc1ccccc1 ; CC1CC(c2nncn2C)CCN1]

### Methyl 2-methylpiperidine-4-carboxylate

Methyl 2-methylpyridine-4-carboxylate (15.0 g, 96.3 mmol, 97% purity) is dissolved in glacial acetic acid (100 mL). PtO₂ (700 mg) is added, and the reaction mixture is hydrogenated at 60° C. for 8 h under 60 psi of H₂. The catalyst is filtered, and the filtrate is concentrated and dried on the lyophilisation to afford the titled compound.

| | |
|---|---|
| C₈H₁₅NO₂ | (M = 157.2 g/mol) |
| ESI-MS: | 158 [M + H]⁺ |
| Rt (HPLC): | 0.19 min (method D) |

### Methyl 1-benzyl-2-methylpiperidine-4-carboxylate

Methyl 2-methylpiperidine-4-carboxylate (14.5 g, 92.2 mmol) is dissolved in DMF (70 mL). K₂CO₃ (38.2 g, 277 mmol) is added, and the reaction mixture is stirred at rt for 10 min. Then, it is cooled to 0° C., benzyl bromide (12.3 mL, 101 mmol) is added dropwise, and the reaction mixture is stirred at rt overnight. The reaction mixture is filtered, and the filtrate is concentrated. The residue is diluted with EtOAc and washed with water (2×) and brine (2×). The combined aqueous layers are extracted again with EtOAc (3×). Then the combined organic layers are dried, filtered, and concentrated. The residue is purified by column chromatography (SiO₂, CyH/EtOAc gradient 95:5 to 50:50) to yield the desired compound.

| | |
|---|---|
| C₁₅H₂₁NO₂ | (M = 247.3 g/mol) |
| ESI-MS: | 248 [M + H]⁺ |
| Rt (HPLC): | 0.58 min (method D) |

### 1-Benzyl-2-methylpiperidine-4-carbohydrazide

Methyl 1-benzyl-2-methylpiperidine-4-carboxylate (4.50 g, 18.2 mmol) is dissolved in EtOH (30 mL). Hydrazine monohydrate (60% in water, 7.39 mL, 91.0 mmol) is added and the reaction mixture is refluxed for 15 h. The reaction mixture is concentrated, and the residue is diluted with a small amount of water and extracted with DCM (3×). The combined organic layers are dried, filtered and concentrated to afford the desired compound.

| | |
|---|---|
| C₁₄H₂₁N₃O | (M = 247.3 g/mol) |
| ESI-MS: | 248 [M + H]⁺ |
| Rt (HPLC): | 0.74 min (method C) |

### 1-Benzyl-2-methyl-4-(4-methyl-4H-1,2,4-triazol-3-yl)piperidine

1-Benzyl-2-methylpiperidine-4-carbohydrazide (5.25 g, 19.1 mmol, 90% purity) is dissolved in 1.4-dioxane (50 mL). N,N-dimethylformamide dimethyl acetal (6.34 mL, 47.8 mmol) is added and the reaction mixture is stirred at 50° C. for 1.5 h. After cooling to 0° C., glacial acetic acid (5.46 mL, 95.5 mmol) and methylamine (2 M in THF, 47.8 mL, 95.5 mmol) are added dropwise, and the reaction mixture is stirred at 70° C. for 18 h. The reaction mixture is concentrated, the residue is diluted with EtOAc (300 mL) and washed with a 2 M K₂CO₃ solution (3×100 mL) and brine (2×). The organic layer is separated, dried, and concentrated to yield the desired compound.

| | |
|---|---|
| C₁₆H₂₂N₄ | (M = 270.4 g/mol) |
| ESI-MS: | 271 [M + H]⁺ |
| Rt (HPLC): | 0.46 min (method D) |

### Intermediate I.2: 2-methyl-4-(4-methyl-4H-1,2,4-triazol-3-yl)piperidine

1-Benzyl-2-methyl-4-(4-methyl-4H-1,2,4-triazol-3-yl)piperidine (3.21 g, 11.9 mmol) is dissolved in EtOH (40 mL). Pd/C 10% (400 mg) is added, and the reaction mixture is hydrogenated at 60° C. for 3 h under 60 psi of H₂. The catalyst is filtered, and the filtrate is concentrated to afford the titled compound.

| | |
|---|---|
| C₉H₁₆N₄ | (M = 180.3 g/mol) |
| ESI-MS: | 181 [M + H]⁺ |
| Rt (HPLC): | 0.10 min (method D) |

### Synthesis of Intermediate I.4

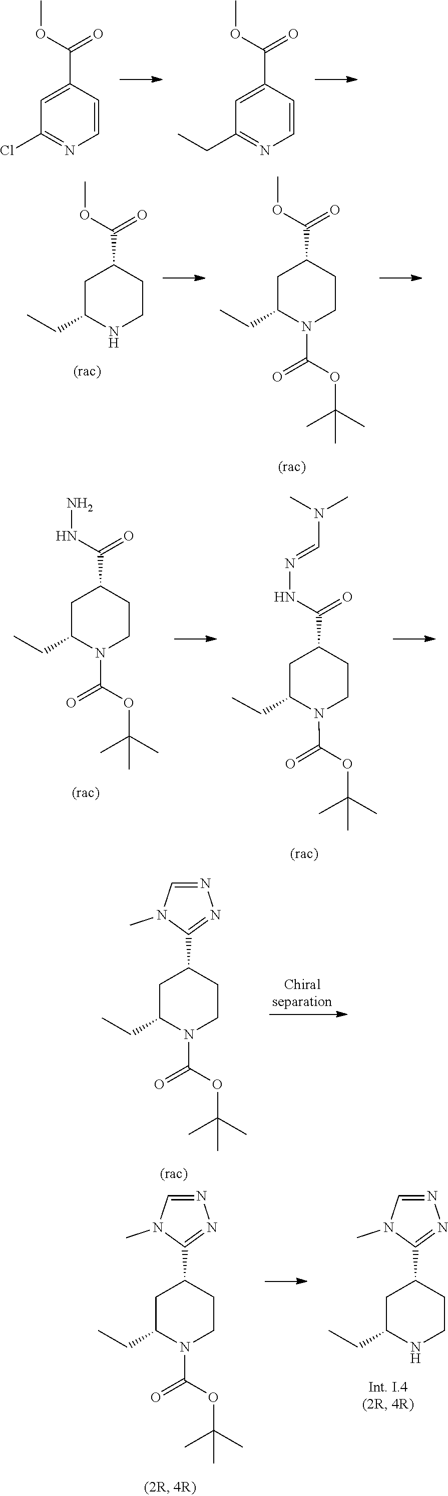

### Methyl 2-ethylpyridine-4-carboxylate

To a solution of methyl 2-chloropyridine-4-carboxylate (10.0 g, 58.3 mmol) in THF (100 mL) is added Tris (acetylacetonato) iron (III) (1.03 g, 2.91 mmol) and EtMgBr (14.13 g, 117 mmol) at 0° C. under N₂. The mixture is stirred at 0° C. for 5 h. The reaction mixture is diluted with an aq. solution of NH₄Cl (100 mL) and extracted with EtOAc (2×100 mL). The combined organic layers are dried over Na₂SO₄, filtered, and concentrated under reduced pressure. The residue is purified by column chromatography (SiO₂, PE/EtOAc gradient 1:0 to 0:1) to afford the desired compound.

| | |
|---|---|
| C₉H₁₁NO₂ | (M = 165.2 g/mol) |
| ESI-MS: | 166 [M + H]⁺ |
| Rt (HPLC): | 0.19 min (method F) |

### (rac)-Methyl cis-2-ethylpiperidine-4-carboxylate

To a solution of methyl 2-ethylpyridine-4-carboxylate (9.00 g, 54.5 mmol) in AcOH (5 mL) is added Pd/C (11.55 g, 109 mmol) at 80° C. under H₂. The mixture is heated to 80° C. and stirred for 16 h. The reaction mixture is diluted with H₂O (50 mL) and extracted with EtOAc (2×30 mL). The combined organic layers are dried over Na₂SO₄, filtered, and concentrated under reduced pressure. The residue is purified by column chromatography (SiO₂, PE/EtOAc gradient 0:1 to 1:0) to afford the desired compound.

| | |
|---|---|
| C₉H₁₇NO₂ | (M = 171.2 g/mol) |
| ESI-MS: | 172 [M + H]⁺ |
| Rt (HPLC): | 0.09 min (method F) |

### (rac)-1-tert-Butyl 4-methyl cis-2-ethylpiperidine-1,4-dicarboxylate

To a solution of (rac)-methyl cis-2-ethylpiperidine-4-carboxylate (9.00 g, 52.6 mmol) in DCM (100 mL) is added di-tert-butyl dicarbonate (11.70 g, 53.6 mmol) at 20° C. under N₂. The mixture is stirred at 20° C. for 12 h. The reaction mixture is diluted with H₂O (50 mL) and extracted with EtOAc (2×30 mL). The combined organic layers are dried over Na₂SO₄, filtered, and concentrated under reduced pressure. The residue is purified by column chromatography (SiO₂, PE/EtOAc gradient 0:1 to 1:0) to afford the desired compound.

| | |
|---|---|
| C₁₄H₂₅NO₄ | (M = 271.4 g/mol) |
| ESI-MS: | 172 [M + H-BOC]⁺ |
| Rt (HPLC): | 0.45 min (method H) |

### (rac)-tert-Butyl cis-2-ethyl-4-(hydrazinecarbonyl)piperidine-1-carboxylate

To a solution of (rac)-1-tert-butyl 4-methyl cis-2-ethylpiperidine-1,4-dicarboxylate (10.0 g, 36.9 mmol) in EtOH (50 mL) is added hydrazine monohydrate (50 mL) at 20° C. under N₂. The mixture is stirred at 20° C. for 12 h. The reaction mixture is diluted with H₂O (200 mL) and extracted with EtOAc (2×100 mL). The combined organic layers are dried over Na₂SO₄, filtered, and concentrated under reduced pressure. The residue is purified by column chromatography (SiO₂, PE/EtOAc gradient 0:1 to 1:0) to afford the desired compound.

| | |
|---|---|
| C₁₃H₂₅N₃O₃ | (M = 271.4 g/mol) |
| ESI-MS: | 216 [M + H-isobuten]⁺ |
| Rt (HPLC): | 0.47 min (method H) |

### (rac)-tert-Butyl cis-4-{N′-[(E)-(dimethylamino)methylidene]hydrazinecarbonyl}-2-ethylpiperidine-1-carboxylate

To a solution of (rac)-tert-butyl cis-2-ethyl-4-(hydrazinecarbonyl)piperidine-1-carboxylate (9.00 g, 33.2 mmol) in 1.4-dioxane (2 mL) is added 1,1-dimethoxy-N,N-dimethyl-methanamine (10.0 g, 84.2 mmol) at 20° C. under N₂. The mixture is heated to 50° C. and stirred for 2 h. After cooling to ambient temperature, the mixture is concentrated and used directly in the next step.

| | |
|---|---|
| C₁₆H₃₀N₄O₃ | (M = 326.4 g/mol) |
| Rf | 0.5 |
| (TLC on silica; PE/EtOAc 3/1): | |

### (rac)-tert-Butyl cis-2-ethyl-4-(4-methyl-4H-1,2,4-triazol-3-yl)piperidine-1-carboxylate

To a solution of (rac)-tert-butyl cis-4-{N′-[(E)-(dimethylamino)methylidene]hydrazinecarbonyl}-2-ethylpiperidine-1-carboxylate (9.00 g, 27.6 mmol) in 1.4-dioxane (50 mL) is added methyl amine (7.71 g, 248 mmol) at 50° C. under N₂. The mixture is heated to 70° C. and stirred for 12 h. After cooling to ambient temperature, the reaction mixture is diluted with H₂O (200 mL) and extracted with EtOAc (2×100 mL). The combined organic layers are dried over Na₂SO₄, filtered, and concentrated under reduced pressure. The residue is purified by column chromatography (SiO₂, PE/EtOAc gradient 1:0 to 0:1) to afford the desired compound.

| | |
|---|---|
| C₁₅H₂₆N₄O₂ | (M = 294.4 g/mol) |
| ESI-MS: | 239 [M + H-isobuten]⁺ |
| Rt (HPLC): | 0.49 min (method H) |

### tert-Butyl (2R,4R)-2-ethyl-4-(4-methyl-4H-1,2,4-triazol-3-yl)piperidine-1-carboxylate

(rac)-tert-butyl cis-2-ethyl-4-(4-methyl-4H-1,2,4-triazol-3-yl)piperidine-1-carboxylate (4.50 g, 15.3 mmol) is separated by chiral SFC (Instrument: Waters SFC350 preparative SFC; Column: DAICEL CHIRALPAK IH (250 mm*50 mm, 10 um); Mobile phase: A for CO2 and B for IPA (0.1% NH3H2O); Gradient: B %=20% isocratic elution mode; Flow rate: 200 g/min; Wavelength: 220 nm; Column temperature: 40° C.; System back pressure: 100 bar) to afford the desired compound.

| | |
|---|---|
| C₁₅H₂₆N₄O₂ | (M = 294.4 g/mol) |
| ESI-MS: | 239 [M + H-isobuten]⁺ |
| Rt (SFC): | 3.31 min (method I) |

The enantiomer (tert-Butyl (2S,4S)-2-ethyl-4-(4-methyl-4H-1,2,4-triazol-3-yl)piperidine-1-carboxylate) elutes with a retention time of 3.25 min (SFC, method I).

### (2R,4R)-2-ethyl-4-(4-methyl-4H-1,2,4-triazol-3-yl)piperidine hydrochloride

Tert-butyl (2R,4R)-2-ethyl-4-(4-methyl-4H-1,2,4-triazol-3-yl)piperidine-1-carboxylate (2.00 g, 6.79 mmol) is dissolved in HCl/EtOAc (20 mL), and stirred at 15° C. for 2 h. The reaction mixture is concentrated under reduced pressure to afford the desired compound.

| | |
|---|---|
| C₁₀H₁₈N₄ | (M = 194.3 g/mol) |
| ESI-MS: | 195 [M+H]+ |
| Rt (HPLC): | 1.72 min (method J) |

### Intermediate I.4: (2R,4R)-2-ethyl-4-(4-methyl-4H-1,2,4-triazol-3-yl)piperidine

(2R,4R)-2-ethyl-4-(4-methyl-4H-1,2,4-triazol-3-yl)piperidine hydrochloride (400 mg, 1.73 mmol) is dissolved in MeOH, ammonia in MeOH 7 M is added and it is filtered through a 10 g column with an amino phase (DCM/MeOH gradient 85/15; 100 mL). The desired fractions are combined and concentrated to dryness to afford the desired compound.

C₁₀H₁₈N₄ (M=194.3 g/mol)

ESI-MS: 195 [M+H]⁺

Rt (HPLC): 0.24 min (method B)

### Synthesis of Intermediate I.5

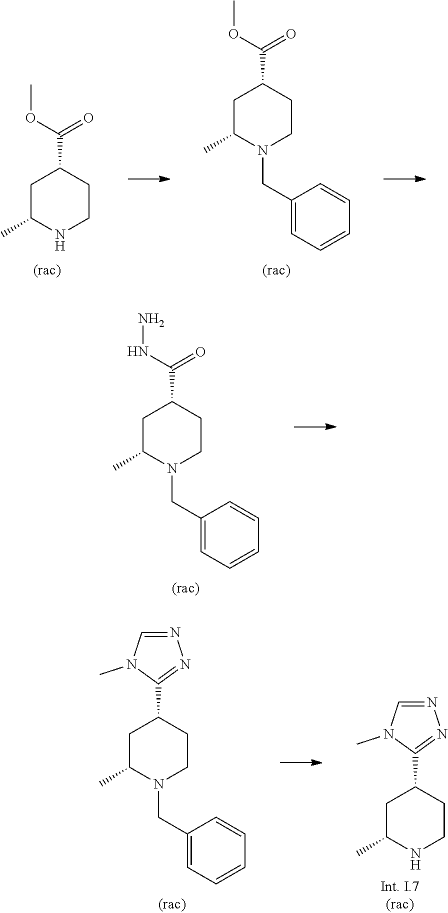 [SMILES: COC(=O)[C@@H]1CCN[C@H](C)C1 ; COC(=O)[C@@H]1CCN(Cc2ccccc2)[C@H](C)C1 ; C[C@@H]1C[C@H](C(=O)NN)CCN1Cc1ccccc1 ; C[C@@H]1C[C@H](c2nncn2C)CCN1Cc1ccccc1 ; C[C@@H]1C[C@H](c2nncn2C)CCN1]

### (rac)-Methyl cis-1-benzyl-2-methylpiperidine-4-carboxylate

(rac)-Methyl cis-2-methylpiperidine-4-carboxylate hydrochloride (1.00 g, 4.91 mmol, 95% purity) is dissolved in DMF (5 mL). K₂CO₃ (2.71 g, 19.6 mmol) is added, and the reaction mixture is stirred at rt for 5 min. Then benzyl bromide (0.60 mL, 4.93 mmol) is added dropwise, and the reaction mixture is stirred at rt for 24 h. The reaction mixture is filtered, and the filtrate is diluted with water and extracted with EtOAc. The organic layer is dried, filtered, and concentrated to yield the desired compound.

| | |
|---|---|
| C₁₅H₂₁NO₂ | (M = 247.3 g/mol) |
| ESI-MS: | 248 [M + H]⁺ |
| Rt (HPLC): | 0.56 min (method D) |

### (rac)-cis-1-Benzyl-2-methylpiperidine-4-carbohydrazide

(rac)-Methyl cis-1-benzyl-2-methylpiperidine-4-carboxylate (1.20 g, 4.85 mmol) is dissolved in EtOH (10 mL). Hydrazine monohydrate (80%, 1.48 mL, 24.3 mmol) is added and the reaction mixture is refluxed for 15 h. The reaction mixture is concentrated, and the residue is diluted with a small amount of water and extracted with DCM. The organic layer is dried, filtered and concentrated to afford the desired compound.

| | |
|---|---|
| C₁₄H₂₁N₃O | (M = 247.3 g/mol) |
| ESI-MS: | 248 [M + H]⁺ |
| Rt (HPLC): | 0.37 min (method D) |

### (rac)-cis-1-Benzyl-2-methyl-4-(4-methyl-4H-1,2,4-triazol-3-yl)piperidine

(rac)-cis-1-Benzyl-2-methylpiperidine-4-carbohydrazide (1.18 g, 4.77 mmol) is dissolved in 1.4-dioxane (15 mL). N,N-dimethylformamide dimethyl acetal (1.58 mL, 11.9 mmol) is added and the reaction mixture is stirred at 50° C. for 1.5 h. After cooling to rt, Methylamine (2 M in THF, 11.9 mL, 23.9 mmol) and glacial acetic acid (1.36 mL, 23.9 mmol) are added dropwise, and the reaction mixture is stirred at 90° C. for 18 h. The reaction mixture is concentrated, the residue is diluted with EtOAc (50 mL) and washed with a 2 M K₂CO₃ solution (3×30 mL) and brine (2×). The organic layer is separated, dried, and concentrated to yield the desired compound.

| | |
|---|---|
| C₁₆H₂₂N₄ | (M = 270.4 g/mol) |
| ESI-MS: | 271 [M + H]+ |
| Rt (HPLC): | 0.47 min (method D) |

### Intermediate I.5: (rac)-cis-2-methyl-4-(4-methyl-4H-1,2,4-triazol-3-yl)piperidine

(rac)-cis-1-Benzyl-2-methyl-4-(4-methyl-4H-1,2,4-triazol-3-yl)piperidine (1.28 g, 4.73 mmol) is dissolved in EtOH (20 mL). Pd/C 10% (130 mg) is added, and the reaction mixture is hydrogenated at 60° C. overnight under 60 psi of H₂. The catalyst is filtered, and the filtrate is concentrated. The residue is purified by column chromatography (SiO₂, DCM/MeOH+NH₃ gradient) to afford the desired compound.

| | |
|---|---|
| C₉H₁₆N₄ | (M = 180.3 g/mol) |
| ESI-MS: | 181 [M + H]⁺ |
| Rt (HPLC): | 0.10 min (method D) |

### Synthesis of Intermediate I.6

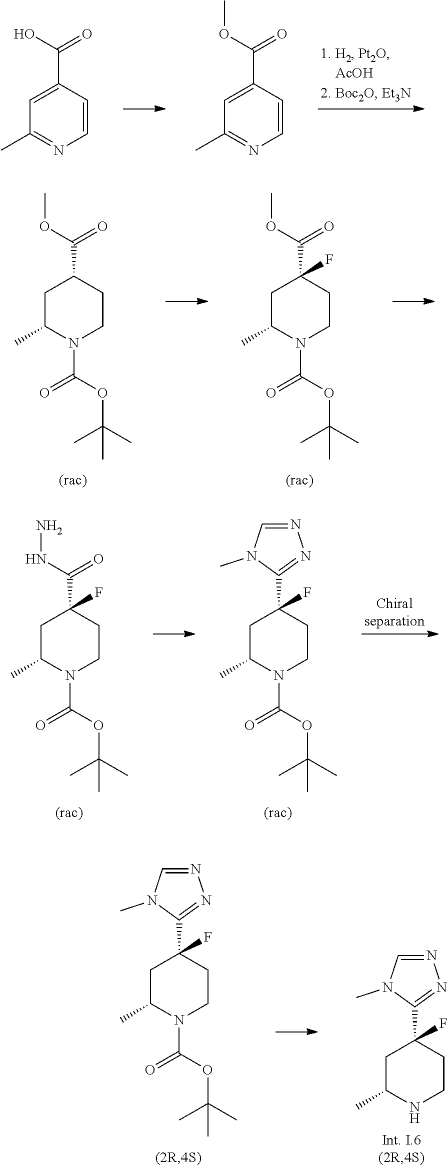

### Methyl 2-methylpyridine-4-carboxylate

2-Methylpyridine-4-carboxylic acid (500 g, 3.63 mol) is dissolved in MeOH (3.00 L). At ambient temperature, H₂SO₄ (750 mL) is added dropwise, and the reaction mixture is stirred at 60° C. for 2 h. After cooling to ambient temperature, the reaction mixture is quenched by the addition of an aq. NaHCO₃ solution (5.00 L, 10%) at 0-5° C., and extracted with DCM (3×1.50 L) and washed with brine. The combined organic layers are dried over Na₂SO₄, filtered, and concentrated to obtain the desired compound as a crude.

Rf (TLC on silica; PE/EtOAc 5/1): 0.48 ¹H NMR (400 MHz, CDCl₃) δ ppm 8.58 (d, J=4.8 Hz, 1H), 7.65 (s, 1H), 7.57 (d, J=4.4 Hz, 1H), 3.89 (s, 3H), 2.57 (s, 3H).

### (rac)-1-tert-Butyl 4-methyl cis-2-methylpiperidine-1,4-dicarboxylate

Methyl 2-methylpyridine-4-carboxylate (100 g, 0.66 mol) is dissolved in AcOH (700 mL). At 10˜25° C., PtO₂ (15.0 g, 0.06 mol) is added, and the reaction mixture is hydrogenated at 45˜50° C. for 6 h under 50 psi of H₂. The catalyst is filtered, and the filtrate is concentrated in vacuum to afford (rac)-methyl cis-2-methylpiperidine-4-carboxylate, which is used without further purification. The obtained residue is dissolved in DCM (2.80 L) under N₂. Boc₂O (666 g, 3.00 mol) and TEA (384 g, 3.81 mol) are added at ambient temperature ° C., and the reaction mixture is stirred at the same temperature for 12 h. The reaction mixture is diluted with H₂O (1.20 L), extracted with EtOAc (3×1.20 L) and washed with brine. The combined organic layers are dried over Na₂SO₄, filtered, and concentrated. The residue is purified by column chromatography (SiO₂, PE/EtOAc gradient 100:1 to 0:1) to give the desired product.

| | |
|---|---|
| C₁₃H₂₂FNO₄ | (M = 275.3 g/mol) |
| ESI-MS: | 261.1 [M − CH₃]⁺ |
| Rt (HPLC): | 0.75 min (method G) |

### (rac)-1-tert-Butyl 4-methyl cis-4-fluoro-2-methylpiperidine-1,4-dicarboxylate

(rac)-1-tert-Butyl 4-methyl cis-2-methylpiperidine-1,4-dicarboxylate (180 g, 0.70 mol) is dissolved in THF (900 mL) under N₂. NFSI (275 g, 0.87 mol) and LDA (2 M in THF, 489 mL, 0.98 mol) are added at −65˜−60° C., and the reaction mixture is stirred at −65˜−60° C. for 2 h. The reaction mixture is quenched by the addition of H₂O (540 mL), allowed to warm to ambient temperature and extracted with EtOAc (540 mL). The organic layer is washed with brine, dried over Na₂SO₄, filtered, and concentrated. The residue is purified by column chromatography (SiO₂, PE/EtOAc gradient 1:0 to 0:1) to give the desired product.

| | |
|---|---|
| C₁₃H₂₃NO₄ | (M = 257.3 g/mol) |
| ESI-MS: | 243.1 [M − CH₃]⁺ |
| Rt (HPLC): | 0.74 min (method G) |

### (rac)-tert-Butyl cis-4-fluoro-4-(hydrazinecarbonyl)-2-methylpiperidine-1-carboxylate

(rac)-1-tert-Butyl 4-methyl cis-4-fluoro-2-methylpiperidine-1,4-dicarboxylate (170 g, 0.61 mol) is dissolved in EtOH (680 mL) under N₂. Hydrazine monohydrate (80%, 77.3 g, 1.23 mol) is added and the reaction mixture is stirred at 50° C. for 12 h. After cooling to ambient temperature, the reaction mixture is diluted with H₂O (510 mL) and extracted with EtOAc (510 mL). The organic layer is washed with brine, dried over Na₂SO₄, filtered, and concentrated to afford the desired crude compound, which is used without further purification.

¹H NMR (400 MHz, D₂O) δ ppm 1.26 (br s, 3H), 1.48 (s, 9H), 1.91-2.04 (m, 2H), 2.15-2.49 (m, 2H), 3.05-3.25 (m, 1H), 3.87-3.97 (m, 1H), 4.05-4.27 (m, 1H), 7.36-7.84 (m, 1H).

### (rac)-tert-Butyl cis-4-fluoro-2-methyl-4-(4-methyl-4H-1,2,4-triazol-3-yl)piperidine-1-carboxylate

(rac)-tert-Butyl cis-4-fluoro-4-(hydrazinecarbonyl)-2-methylpiperidine-1-carboxylate (210 g, 0.76 mol) is dissolved in 1.4-dioxane (1.44 L) under N₂. DMF-DMA (227 g, 1.91 mol) is added at 15˜25° C., and the reaction mixture is stirred at 50° C. for 1 h. Then methyl amine (552 g, 5.34 mol) and AcOH (320 g, 5.34 mol) are added at 15˜25° C., and it is stirred at 90° C. for 12 h. The reaction mixture is diluted with H₂O (630 mL) and extracted with EtOAc (630 mL). The organic layer is washed with brine, dried over Na₂SO₄, filtered, and concentrated to afford the desired compound as a crude.

| | |
|---|---|
| C₁₄H₂₃FN₄O₂ | (M = 298.4 g/mol) |
| ESI-MS: | 299.2 [M + H]⁺ |
| Rt (HPCL): | 0.87 min (method F) |

### tert-Butyl (2S,4R)-4-fluoro-2-methyl-4-(4-methyl-4H-1,2,4-triazol-3-yl)piperidine-1-carboxylate

(rac)-tert-butyl cis-4-fluoro-2-methyl-4-(4-methyl-4H-1,2,4-triazol-3-yl)piperidine-1-carboxylate (50.0 g, 0.16 mol) is separated by chiral SFC separation (method K) to afford the desired chiral compound.

Rt (SFC): 2.06 min (method K)

The enantiomer tert-Butyl (2R,4S)-4-fluoro-2-methyl-4-(4-methyl-4H-1,2,4-triazol-3-yl)piperidine-1-carboxylate elutes with a retention time of 1.87 min (method K).

### Intermediate I.6: (2S,4R)-4-fluoro-2-methyl-4-(4-methyl-4H-1,2,4-triazol-3-yl)piperidine hydrochloride

Tert-butyl (2S,4R)-4-fluoro-2-methyl-4-(4-methyl-4H-1,2,4-triazol-3-yl)piperidine-1-carboxylate (16.0 g, 53.6 mmol) is dissolved in MeOH (32.0 mL) under N₂. HCl/MeOH (80 mL) is added at 15˜25° C., and it is stirred at 25° C. for 12 h. The reaction mixture is concentrated in vacuum to afford the desired compound.

| | |
|---|---|
| C₉H₁₅FN₄ | (M = 198.2 g/mol) |
| ESI-MS: | 199 [M + H]⁺ |
| Rt (HPLC): | 4.50 min (method L) |

### Synthesis of Intermediate I.7

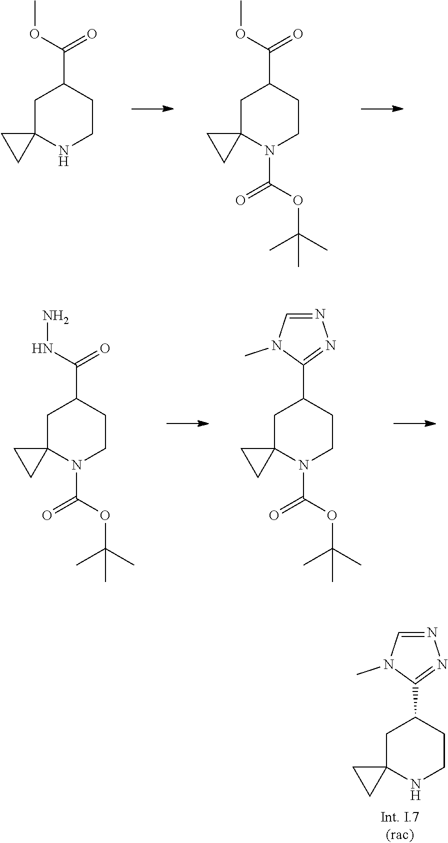 [SMILES: COC(=O)C1CCNC2(CC2)C1 ; COC(=O)C1CCN(C(=O)OC(C)(C)C)C2(CC2)C1 ; CC(C)(C)OC(=O)N1CCC(C(=O)NN)CC12CC2 ; Cn1cnnc1C1CCN(C(=O)OC(C)(C)C)C2(CC2)C1 ; Cn1cnnc1[C@@H]1CCNC2(CC2)C1]

### 4-tert-Butyl 7-methyl 4-azaspiro[2.5]octane-4,7-dicarboxylate

Methyl 4-azaspiro[2.5]octane-7-carboxylate hydrochloride (1.00 g, 4.86 mmol) is suspended in EtOAc and TEA (775 μL, 5.59 mmol). Di-tert-butyl dicarbonate (1.17 g, 5.35 mmol) is added, and the reaction mixture is stirred at rt over the weekend. The reaction mixture is washed with water (2×), 1 M HCl (1×) and brine (1×). The organic layer is dried, filtered, and concentrated to afford the desired compound.

| | |
|---|---|
| C₁₄H₂₃NO₄ | (M = 269.3 g/mol) |
| ESI-MS: | 270 [M + H]⁺ |
| Rt (HPLC): | 0.70 min (method B) |

### tert-Butyl 7-(hydrazinecarbonyl)-4-azaspiro[2.5]octane-4-carboxylate

4-Tert-butyl 7-methyl 4-azaspiro[2.5]octane-4,7-dicarboxylate (1.18 g, 4.38 mmol) is dissolved in EtOH (6 mL). Hydrazine monohydrate (1.10 mL, 22.1 mmol) is added, and the reaction mixture is stirred at 60° C. for 18 h. The reaction mixture is concentrated to afford the desired compound.

| | |
|---|---|
| C₁₃H₂₃N₃O₃ | (M = 269.3 g/mol) |
| ESI-MS: | 270 [M + H]⁺ |
| Rt (HPLC): | 0.47 min (method B) |

### Tert-butyl 7-(4-methyl-4H-1,2,4-triazol-3-yl)-4-azaspiro[2.5]octane-4-carboxylate

Tert-butyl 7-(hydrazinecarbonyl)-4-azaspiro[2.5]octane-4-carboxylate (1.27 g, 4.24 mmol, 90% purity) is dissolved in 1.4-dioxane (12 mL). N,N-dimethylformamide dimethyl acetal (1.41 mL, 10.6 mmol) is added and the reaction mixture is stirred at 50° C. for 2 h. After cooling to rt, glacial acetic acid (1.21 mL, 21.2 mmol) and methylamine (2 M in THF, 10.6 mL, 21.2 mmol) are added dropwise, and the reaction mixture is stirred at 70° C. for 24 h. The reaction mixture is concentrated, the residue is diluted with DCM and washed with water. The organic layer is separated, dried, and concentrated. The residue is purified by preparative HPLC (XBridge C18, ACN/water gradient containing 0.1% NH₃) to afford the desired compound.

| | |
|---|---|
| C₁₅H₂₄N₄O₂ | (M = 292.4 g/mol) |
| ESI-MS: | 293 [M + H]⁺ |
| Rt (HPLC): | 0.51 min (method B) |

### Intermediate I.7: 7-(4-methyl-4H-1,2,4-triazol-3-yl)-4-azaspiro[2.5]octane

Tert-butyl 7-(4-methyl-4H-1,2,4-triazol-3-yl)-4-azaspiro[2.5]octane-4-carboxylate (400 mg, 1.37 mmol) is dissolved in DCM (10 mL). HCl 4 M in 1.4-dioxane (3.42 mL, 13.7 mmol) is added, and the reaction mixture is stirred at rt overnight. The reaction mixture is concentrated, the residue is dissolved in ammonia in MeOH, and filtered through a 10 g column of an amino phase (DCM/MeOH 80/20 gradient) to yield the free base.

| | |
|---|---|
| C₁₀H₁₆N₄ | (M = 192.3 g/mol) |
| ESI-MS: | 193 [M + H]⁺ |
| Rt (HPLC): | 0.18 min (method B) |

### Synthesis of Intermediate II.1

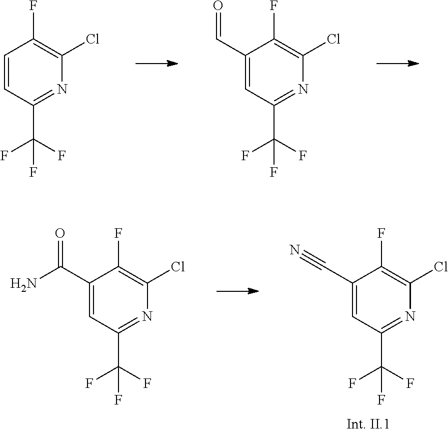 [SMILES: Fc1ccc(C(F)(F)F)nc1Cl ; O=Cc1cc(C(F)(F)F)nc(Cl)c1F ; NC(=O)c1cc(C(F)(F)F)nc(Cl)c1F ; N#Cc1cc(C(F)(F)F)nc(Cl)c1F]

### 2-Chloro-3-fluoro-6-(trifluoromethyl)pyridine-4-carbaldehyde

Under an argon atmosphere, 2-chloro-3-fluoro-6-(trifluoromethyl)pyridine (14.2 g, 69.7 mmol) is added to THF (330 mL), and the resulting mixture is cooled to −75° C. A solution of lithium diisopropylamide (1 M in THF, 77.0 mL, 77.0 mmol) is added dropwise in a period of 90 min, and the mixture is stirred for 60 min at −75° C. DMF (6.44 mL, 83.7 mmol) is added dropwise. The mixture is stirred for additional 30 min at −75° C. The reaction is quenched by addition of a half concentrated acetic acid (40 mL) and diluted with water and EtOAc. The organic phase is separated, washed with brine, dried, and concentrated. The residue is purified by column chromatography (SiO₂, CyH/EtOAc gradient 100:0 to 85:15) to yield the desired compound.

| | |
|---|---|
| C₇H₂ClF₄NO | (M = 227.5 g/mol) |
| EI-MS: | 227 M*+ |
| Rt (HPLC): | 0.48 min (method B) |

### 2-Chloro-3-fluoro-6-(trifluoromethyl)pyridine-4-carboxamide

Ammonium acetate (47.1 g, 611 mmol) and 2-chloro-3-fluoro-6-(trifluoromethyl)pyridine-4-carbaldehyde (13.9 g, 61.1 mmol) are mixed, and acetonitrile (300 mL) is added. Phenyltrimethylammonium tribromide (47.4 g, 122 mmol) is added in small portions, and the resulting reaction mixture is stirred at ambient temperature overnight. The mixture is filtered through a pad of silica, and the residue is washed with acetonitrile, purified by column chromatography (SiO₂, CyH/EtOAc gradient 1:0 to 7:3) to yield the desired product.

| | |
|---|---|
| C₇H₃ClF₄N₂O | (M = 242.6 g/mol) |
| ESI-MS: | 241 [M − H]⁻ |
| Rt (HPLC): | 0.47 min (method B) |

### Intermediate II.1: 2-chloro-3-fluoro-6-(trifluoromethyl)pyridine-4-carbonitrile

Product of the previous step, 2-chloro-3-fluoro-6-(trifluoromethyl)pyridine-4-carboxamide (5.10 g, 21.0 mmol) is suspended in dichloromethane (400 mL), and Burgess reagent (8.75 g, 35.6 mmol) is added at ambient temperature. The resulting reaction mixture is stirred for 40 h and then directly purified by column chromatography (SiO₂, CyH/EtOAc gradient 100:0 to 95:5) to yield the titled compound.

| | |
|---|---|
| C₇HClF₄N₂ | (M = 224.5 g/mol) |
| ESI-MS: | 225 [M + H]⁺ |
| Rt (HPLC): | 0.57 min (method A) |

### Synthesis of Intermediate II.2

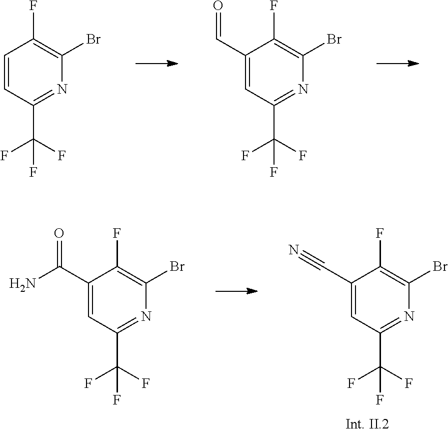 [SMILES: Fc1ccc(C(F)(F)F)nc1Br ; O=Cc1cc(C(F)(F)F)nc(Br)c1F ; NC(=O)c1cc(C(F)(F)F)nc(Br)c1F ; N#Cc1cc(C(F)(F)F)nc(Br)c1F]

### 2-Bromo-3-fluoro-6-(trifluoromethyl)pyridine-4-carbaldehyde

Under an argon atmosphere, 2-bromo-3-fluoro-6-(trifluoromethyl)pyridine (3.08 g, 12.6 mmol) is added to THF (75 mL), and the resulting mixture is cooled to −70° C. A solution of lithium diisopropylamide (1 M in THF, 13.9 mL, 13.9 mmol) is added dropwise, and the mixture is stirred for 90 min at −70° C. DMF (1.17 mL, 15.1 mmol) is added dropwise. The mixture is stirred for additional 30 min at −70° C. The reaction is quenched by addition of a half concentrated acetic acid (800 μL) and diluted with water and EtOAc. The organic phase is separated, dried over MgSO₄, and concentrated. The residue is purified by column chromatography (SiO₂, CyH/EtOAc gradient 1:0 to 4:1) to yield 2-bromo-3-fluoro-6-(trifluoromethyl)pyridine-4-carbaldehyde.

| | |
|---|---|
| C₇H₂BrF₄NO | (M = 272.0 g/mol) |
| ESI-MS: | no mass detected |
| Rt (HPLC): | 0.51 min (method B) |

### 2-Bromo-3-fluoro-6-(trifluoromethyl)pyridine-4-carboxamide

Ammonium acetate (5.53 g, 71.8 mmol) and 2-bromo-3-fluoro-6-(trifluoromethyl)pyridine-4-carbaldehyde (2.17 g, 7.18 mmol) are mixed, and acetonitrile (44 mL) is added. Phenyltrimethylammonium tribromide (5.57 g, 14.4 mmol) is added in small portions, and the resulting reaction mixture is stirred for 72 h at ambient temperature. The mixture is filtered, and the residue is washed with acetonitrile, purified by column chromatography (dry load with Celite®, SiO₂, CyH/EtOAc gradient 1:0 to 7:3) to yield the desired product.

| | |
|---|---|
| C₇H₃BrF₄N₂O | (M = 287.0 g/mol) |
| ESI-MS: | 285/287 [M − H]⁻ |
| Rt (HPLC): | 0.50 min (method B) |

### Intermediate II.2: 2-bromo-3-fluoro-6-(trifluoromethyl)pyridine-4-carbonitrile

Product of the previous step, 2-bromo-3-fluoro-6-(trifluoromethyl)pyridine-4-carboxamide (975 mg, 3.40 mmol) is suspended in dichloromethane (80 mL), and Burgess reagent (1.25 g, 5.10 mmol) is added at ambient temperature. The resulting reaction mixture is stirred for 40 h and then directly purified by column chromatography (dry load, SiO₂, CyH/EtOAc gradient 1:0 to 9:1) to yield the title compound.

| | |
|---|---|
| C₇HBrF₄N₂ | (M = 268.9 g/mol) |
| ESI-MS: | 268/270 [M + H]⁺ |
| Rt (HPLC): | 0.59 min (method A) |

### Synthesis of Intermediate III.1: (rac)-2-chloro-3-[trans-2-cyclopropyl-4-(4-methyl-4H-1,2,4-triazol-3-yl)piperidin-1-yl]-6-(trifluoromethyl)pyridine-4-carbonitrile

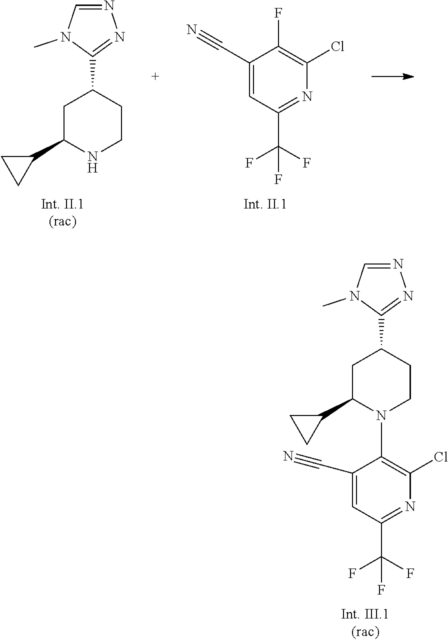 [SMILES: Cn1cnnc1[C@@H]1CCN[C@@H](C2CC2)C1 ; N#Cc1cc(C(F)(F)F)nc(Cl)c1F ; Cn1cnnc1[C@@H]1CCN(c2c(C#N)cc(C(F)(F)F)nc2Cl)[C@@H](C2CC2)C1]

Int. I.1 (500 mg, 2.42 mmol) is dissolved in DMSO (3 mL) and DIPEA (770 μL, 4.45 mmol). At rt, Int. 11.1 (500 mg, 2.23 mmol) is added, and the resulting mixture is stirred at rt for 30 min. The mixture is diluted with water and the formed precipitate is filtered. The residue is diluted with ACN/1H₂O and purified by preparative HPLC (XBridge C18, ACN/water gradient containing 0.1% NH₃) to afford the desired compound.

| | |
|---|---|
| C₁₈H₁₈ClF₃N₆ | (M = 410.8 g/mol) |
| ESI-MS: | 411 [M + H]⁺ |
| Rt (HPLC): | 0.61 min (method B) |

### Intermediates Synthesized Analogous to the Procedure Described for Int. III3

| | | | | Molecular |
|---|---|---|---|---|
| | | | | Formula (MW) |
| | | | | ESI-MS |
| | Starting | | Deviation from | HPLC retention |
| Int. | material | Structure | general procedure | time (method) |
| III.2 (rac cis & trans) | Int. I.2 + Int. II.2 | 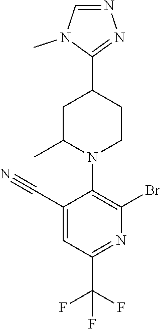 [SMILES: CC1CC(c2nncn2C)CCN1c1c(C#N)cc(C(F)(F)F)nc1Br] | 1.2 eq of Int. I.2 1 h @ 15° C. 1 h @ rt | C₁₆H₁₆BrF₃N₆ (M = 429.2 g/mol) ESI-MS: 429 [M + H]⁺ Rt (HPLC): 0.81 min (method D) |
| III.3 (rac cis & trans) | Int. I.2 + 2-chloro-3- fluoropyridine- 4-carbonitrile | 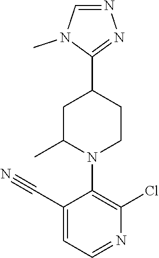 [SMILES: CC1CC(c2nncn2C)CCN1c1c(C#N)ccnc1Cl] | 1.2 eq of Int. I.2 3 d @ rt purification by column chromatography (SiO₂, DCM/MeOH gradient 95:5 to 90:10) | C₁₅H₁₇ClN₆ (M = 316.8 g/mol) ESI-MS: 317 [M + H]⁺ Rt (HPLC): 0.66 min (method D) |
| III.5 (2R, 4R) | Int. I.4 + Int. II.1 | 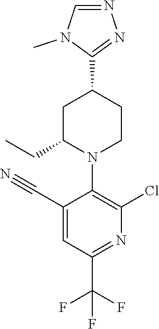 [SMILES: CC[C@@H]1C[C@H](c2nncn2C)CCN1c1c(C#N)cc(C(F)(F)F)nc1Cl] | 2.2 eq of Int. I.4 2h @ rt purification by preparative HPLC (XBridge C18, acetonitrile/water gradient containing 0.1% TFA) | C₁₇H₁₈ClF₃N₆ (M = 398.8 g/mol) ESI-MS: 399 [M + H]⁺ Rt (HPLC): 0.53 min (method A) |
| III.6 (rac)-cis | Int. I.5 + 2-chloro-3- fluoropyridine- 4-carbonitrile | 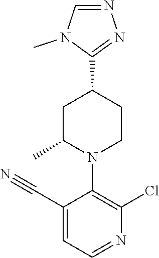 [SMILES: C[C@@H]1C[C@H](c2nncn2C)CCN1c1c(C#N)ccnc1Cl] | 1.2 eq of Int. I.2 3d @ rt 16 h @ 70° C. purification by preparative HPLC (Stablebond C18, acetonitrile/water gradient containing 0.1% TFA) | C₁₅H₁₇ClN₆ (M = 316.8 g/mol) ESI-MS: 317 [M + H]⁺ Rt (HPLC): 0.68 min (method N) |
| III.7 (2R, 4S) | Int. I.6 + 2-chloro-3- fluoropyridine- 4-carbonitrile | 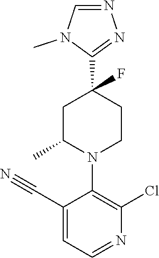 [SMILES: C[C@@H]1C[C@](F)(c2nncn2C)CCN1c1c(C#N)ccnc1Cl] | 1.3 eq of Int. I.2 3 d @ rt 16 h @ 60° C. 8 h @ 90° C. 12 h @ 110° C. | C₁₅H₁₆ClFN₆ (M = 334.8 g/mol) ESI-MS: 335 [M + H]⁺ Rt (HPLC): 0.76 min (method C) |
| III.8 (2R, 4S) | Int. I.6 + Int. II.1 | 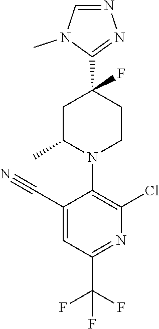 [SMILES: C[C@@H]1C[C@](F)(c2nncn2C)CCN1c1c(C#N)cc(C(F)(F)F)nc1Cl] | 1.3 eq of Int. I.2 1.0 eq DIPEA 16 h @ rt | C₁₆H₁₅ClF₄N₆ (M = 402.8 g/mol) ESI-MS: 403 [M + H]⁺ Rt (HPLC): 0.93 min (method D) |
| III.9 (rac) | Int. I.7 + Int. II.1 | 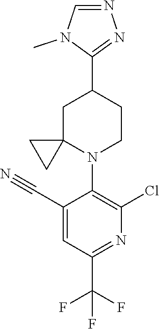 [SMILES: Cn1cnnc1C1CCN(c2c(C#N)cc(C(F)(F)F)nc2Cl)C2(CC2)C1] | 3.5 h @ rt purification by preparative HPLC (Sunfire C18, acetonitrile/water gradient containing 0.1% TFA) | C₁₇H₁₆ClF₃N₆ (M = 396.8 g/mol) ESI-MS: 397 [M + H]⁺ Rt (HPLC): 0.56 min (method A) |

### Synthesis of Intermediate III.4: 2-chloro-3-[(2S,4R)-2-cyclopropyl-4-(4-methyl-4H-1,2,4-triazol-3-yl)piperidin-1-yl]-6-(trifluoromethyl)pyridine-4-carbonitrile

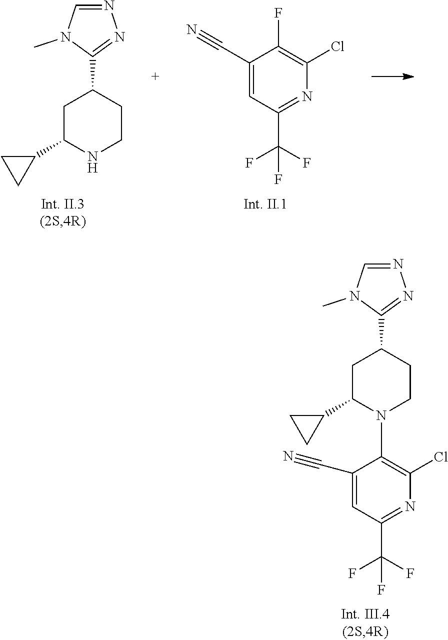 [SMILES: Cn1cnnc1[C@@H]1CCN[C@H](C2CC2)C1 ; N#Cc1cc(C(F)(F)F)nc(Cl)c1F ; Cn1cnnc1[C@@H]1CCN(c2c(C#N)cc(C(F)(F)F)nc2Cl)[C@H](C2CC2)C1]

Int. I.1 (200 mg, 0.97 mmol) is dissolved in DMSO (1 mL) and DIPEA (308 μL, 1.78 mmol). At rt, Int. 11.1 (200 mg, 0.89 mmol) is added, and the resulting mixture is stirred at rt for 20 h. The mixture is diluted with ACN/H₂O/TFA and purified by preparative HPLC (XBridge C18, ACN/water gradient containing 0.1% TFA) to afford the desired compound.

| | |
|---|---|
| C₁₈H₁₈ClF₃N₆ | (M = 410.8 g/mol) |
| ESI-MS: | 411 [M + H]⁺ |
| Rt (HPLC): | 0.54 min (method A) |

### Synthesis of Intermediate IV.1

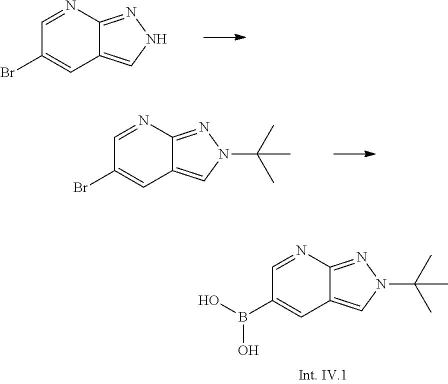 [SMILES: Brc1cnc2n[nH]cc2c1 ; CC(C)(C)n1cc2cc(Br)cnc2n1 ; CC(C)(C)n1cc2cc(B(O)O)cnc2n1]

### 5-Bromo-2-tert-butyl-2H-pyrazolo[3,4-b]pyridine

5-Bromo-2H-pyrazolo[3,4-b]pyridine (4.0 g, 19.8 mmol) is suspended in toluene (23 mL), and tert-butyl acetate (26.6 mL, 198 mmol) is added. Methanesulfonic acid (1.3 mL, 19.8 mmol) is added slowly. The resulting reaction mixture is heated to 80° C. and stirred for 1 h. After cooling to ambient temperature, additional methanesulfonic acid (1.3 mL, 19.8 mmol) is added, and the reaction mixture is heated to 80° C. and stirred for 1 h. After cooling to ambient temperature, the reaction mixture is concentrated and purified by preparative HPLC (XBridge C18, ACN/water gradient containing 0.1% TFA) to yield the 5-bromo-2-tert-butyl-2H-pyrazolo[3,4-b]pyridine.

| | |
|---|---|
| C₁₀H₁₂BrN₃ | (M = 254.1 g/mol) |
| ESI-MS: | 254/256 [M + H]⁺ |
| Rt (HPLC): | 0.50 min (method A) |

### Intermediate IV.1: {2-tert-butyl-2H-pyrazolo[3,4-b]pyridin-5-yl}boronic acid

5-Bromo-2-tert-butyl-2H-pyrazolo[3,4-b]pyridine (1.50 g, 3.87 mmol), bis(pinacolato)diborane (1.21 g, 4.78 mmol), and potassium acetate (763 mg, 7.77 mmol) are added to 1,4-dioxane (15 mL), and the resulting mixture is degassed by passing an argon stream through the mixture for 10 min. [1,1′-Bis-(diphenylphosphino)-ferrocen]-dichloro-palladium(II) dichloromethane complex (Pd(dppf)Cl₂*CH₂Cl₂, CAS: 95464-05-4) (190 mg, 0.232 mmol) is added, and the mixture is degassed for additional 3 min. The mixture is then heated to 110° C. and stirred at this temperature for 4 h. After cooling to ambient temperature, the mixture is concentrated, and the residue is dissolved in an ACN/water mixture, filtered, and purified by preparative HPLC (XBridge C18, ACN/water gradient containing 0.1% TFA) to yield the title compound.

| | |
|---|---|
| C₁₀H₁₄BN₃O₂ | (M = 219.1 g/mol) |
| ESI-MS: | 220 [M + H]⁺ |
| Rt (HPLC): | 0.27 min (method A) |

### Preparation of Final Compounds

### Example 1 and 2

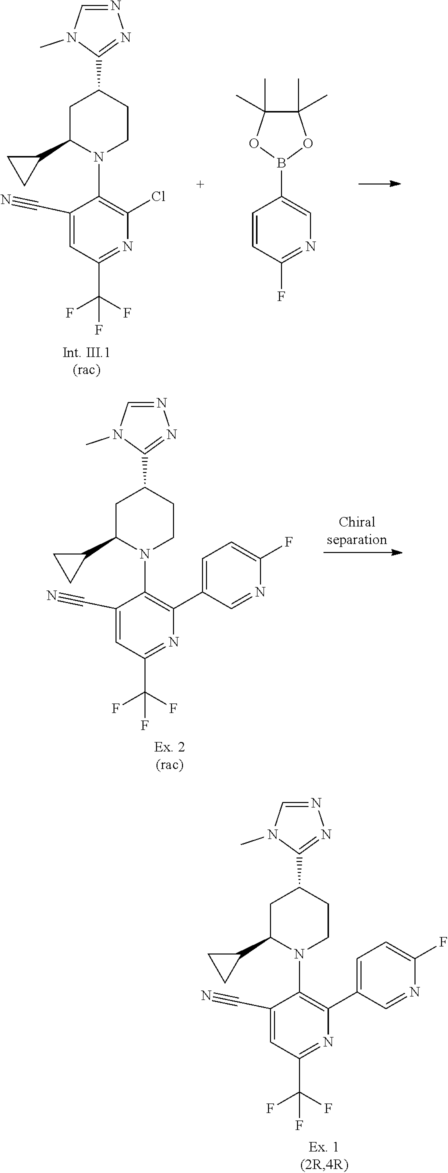

### Example 2: (rac)-3-[trans-2-cyclopropyl-4-(4-methyl-4H-1,2,4-triazol-3-yl)piperidin-1-yl]-6′-fluoro-6-(trifluoromethyl)-[2,3′-bipyridine]-4-carbonitrile

To a solution of Int. 111.1 (50.0 mg, 0.12 mmol) and 2-fluoro-5-(4,4,5,5-tetramethyl-1,3,2-dioxaborolan-2-yl)pyridine (32.6 mg, 0.15 mmol) in 1,4-dioxane (2 mL) is added potassium carbonate (2 M in water, 183 μL, 0.37 mmol). The resulting mixture is purged by passing an argon stream through the solution. Pd(dppf)Cl₂ (8.91 mg, 0.01 mmol) is added, and the mixture is further purged. The reaction mixture is stirred at 95° C. for 1.5 h. After cooling to ambient temperature, the mixture is diluted with ACN, filtered, and purified by preparative HPLC (XBridge C18 column, ACN/water gradient containing 0.1% NH₃) to yield the titled compound.

| | |
|---|---|
| C₂₃H₂₁F₄N₇ | (M = 471.5 g/mol) |
| ESI-MS: | 472 [M + H]⁺ |
| Rt (HPLC): | 0.63 min |
| | (method B) |

¹H NMR (400 MHz, DMSO-d₆) δ ppm 8.64 (d, J=2.3 Hz, 1H), 8.29-8.39 (m, 3H), 7.35 (dd, J=8.5, 2.5 Hz, 1H), 3.83-3.98 (m, 1H), 3.62 (s, 3H), 3.32-3.41 (m, 2H), 2.45 (br dd, J=4.2, 2.3 Hz, 1H), 2.01 (br d, J=12.7 Hz, 1H), 1.54-1.86 (m, 4H), 0.37-0.49 (m, 2H), −0.07-0.00 (m, 1H), −0.25-0.15 (m, 1H).

### Example 1: 3-[(2R,4R)-2-cyclopropyl-4-(4-methyl-4H-1,2,4-triazol-3-yl)piperidin-1-yl]-6′-fluoro-6-(trifluoromethyl)-[2,3′-bipyridine]-4-carbonitrile

(rac)-3-[trans-2-cyclopropyl-4-(4-methyl-4H-1,2,4-triazol-3-yl)piperidin-1-yl]-6′-fluoro-6-(trifluoromethyl)-[2,3′-bipyridine]-4-carbonitrile (35.4 mg, 0.08 mmol) is separated by chiral SFC (method O) to afford the desired chiral compound.

| | |
|---|---|
| C₂₃H₂₁F₄N₇ | (M = 471.5 g/mol) |
| ESI-MS: | 472 [M + H]⁺ |
| Rt (SFC): | 3.46 min |
| | (method O) |

The enantiomer (3-[(2S,4S)-2-cyclopropyl-4-(4-methyl-4H-1,2,4-triazol-3-yl)piperidin-1-yl]-6′-fluoro-6-(trifluoromethyl)-[2,3′-bipyridine]-4-carbonitrile) elutes with a retention time of 2.71 min (method O)

¹H NMR (400 MHz, DMSO-d₆) δ ppm 8.64 (d, J=2.4 Hz, 1H), 8.30-8.38 (m, 3H), 7.35 (dd, J=8.5, 2.5 Hz, 1H), 3.84-3.96 (m, 1H), 3.62 (s, 3H), 3.32-3.40 (m, 2H), 2.40-2.48 (m, 1H), 2.01 (br d, J=12.6 Hz, 1H), 1.54-1.85 (m, 4H), 0.37-0.49 (m, 2H), −0.07-0.00 (m, 1H), −0.25-0.15 (m, 1H).

### Examples Synthesized Analogous to the Procedure Described for Example 2

| | Starting | | Deviation from general |
|---|---|---|---|
| example | materials | Structure | procedure |
| 3 (rac) | Int. III.1 + Int. IV.1 | 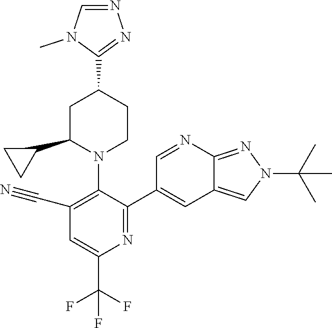 [SMILES: Cn1cnnc1[C@@H]1CCN(c2c(C#N)cc(C(F)(F)F)nc2-c2cnc3nn(C(C)(C)C)cc3c2)[C@@H](C2CC2)C1] | 3.5 eq. K₂CO₃ solution |
| 8 (2S, 4R) | Int. III.4 + 2-fluoro-5- (4,4,5,5- tetramethyl- 1,3,2- dioxaborolan-2- yl)pyridine | 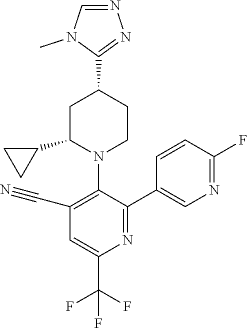 [SMILES: Cn1cnnc1[C@@H]1CCN(c2c(C#N)cc(C(F)(F)F)nc2-c2ccc(F)nc2)[C@H](C2CC2)C1] | 2 h @ 95° C. purification by preparative HPLC (Sunfire C18 column, ACN/water gradient containing 0.1% TFA) |
| 9 (2R, 4R) | Int. III.5 + 2-fluoro-5- (4,4,5,5- tetramethyl- 1,3,2- dioxaborolan-2- yl)pyridine | 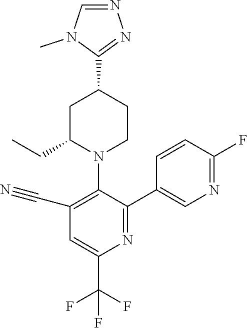 [SMILES: CC[C@@H]1C[C@H](c2nncn2C)CCN1c1c(C#N)cc(C(F)(F)F)nc1-c1ccc(F)nc1] | 3 h @ 95° C. purification by column chromatography (NH₂-SiO₂, DCM/MeOH gradient 100:0 to 98:2) |
| 11 (rac) | Int. III.6 + (6- fluoropyridin-3- yl)boronic acid | 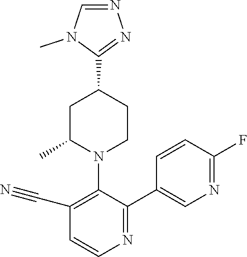 [SMILES: C[C@@H]1C[C@H](c2nncn2C)CCN1c1c(C#N)ccnc1-c1ccc(F)nc1] | 2.0 eq boronic acid 2.5 eq Na₂CO₃ solution 2 h @ 100° C. purification by preparative SFC (Torus_1AA; MEOH/CO2; BPR: 120 bar) |
| 13 (2R, 4S) | Int. III.7 + (6- fluoropyridin-3- yl)boronic acid | 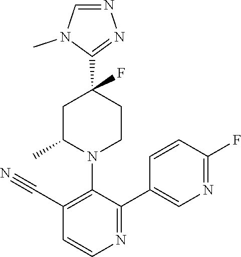 [SMILES: C[C@@H]1C[C@](F)(c2nncn2C)CCN1c1c(C#N)ccnc1-c1ccc(F)nc1] | 3.0 eq Na₂CO₃ solution 4 h @ 100° C. purification by preparative HPLC (Sunfire C18 column, ACN/water gradient containing 0.1% TFA) |
| 14 (2R, 4S) | Int. III.8 + 2-fluoro-5- (4,4,5,5- tetramethyl- 1,3,2- dioxaborolan-2- yl)pyridine | 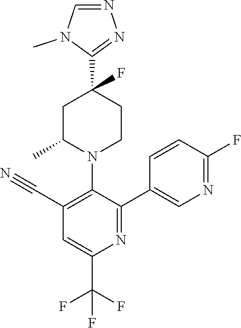 [SMILES: C[C@@H]1C[C@](F)(c2nncn2C)CCN1c1c(C#N)cc(C(F)(F)F)nc1-c1ccc(F)nc1] | 3.0 eq Na₂CO₃ solution 4 h @ 100° C. purification by preparative HPLC (Sunfire C18 column, ACN/water gradient containing 0.1% TFA) |
| 15 (2R, 4S) | Int. III.8 + 4-(4,4,5,5- tetramethyl- 1,3,2- dioxaborolan-2- yl)pyridazine | 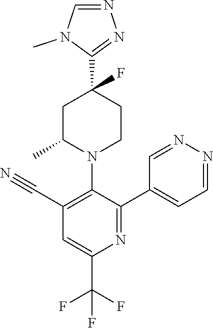 [SMILES: C[C@@H]1C[C@](F)(c2nncn2C)CCN1c1c(C#N)cc(C(F)(F)F)nc1-c1ccnnc1] | 3.0 eq Na₂CO₃ solution 0.2 eq Pd(dppf)Cl₂ 7 h @ 100° C. purification by preparative HPLC (Sunfire C18 column, ACN/water gradient containing 0.1% TFA) |
| 16 (2R, 4S) | Int. III.8 + Int. IV.1 | 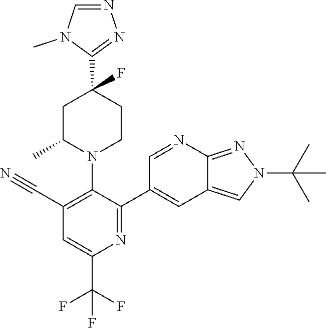 [SMILES: C[C@@H]1C[C@](F)(c2nncn2C)CCN1c1c(C#N)cc(C(F)(F)F)nc1-c1cnc2nn(C(C)(C)C)cc2c1] | 1.0 eq Int. IV.1 3.0 eq Na₂CO₃ solution 4 h @ 100° C. purification by preparative HPLC (Sunfire C18 column, ACN/water gradient containing 0.1% TFA) |
| 17 (rac) | Int. III.9 + 2-fluoro-5- (4,4,5,5- tetramethyl- 1,3,2- dioxaborolan-2- yl)pyridine | 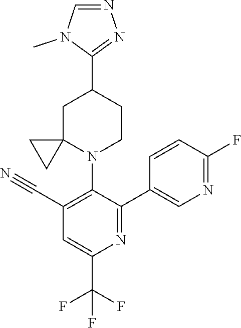 [SMILES: Cn1cnnc1C1CCN(c2c(C#N)cc(C(F)(F)F)nc2-c2ccc(F)nc2)C2(CC2)C1] | 8 h @ 50° C. |

### Example 4 and 10

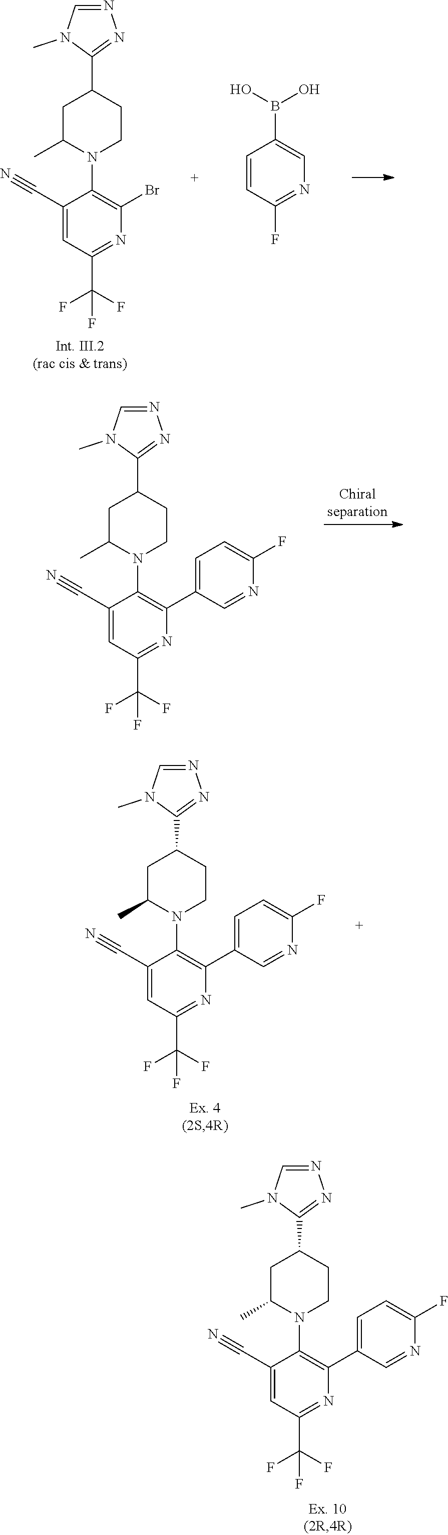

### 6′-Fluoro-3-[2-methyl-4-(4-methyl-4H-1,2,4-triazol-3-yl)piperidin-1-yl]-6-(trifluoromethyl)-[2,3′-bipyridine]-4-carbonitrile

To a solution of Int. III.2 (40.0 mg, 0.09 mmol) and (6-fluoropyridin-3-yl)boronic acid (26.8 mg, 0.19 mmol) in 1,4-dioxane (1 mL) is added sodium carbonate (2 M in water, 0.14 mL, 0.28 mmol). The resulting mixture is purged by passing an argon stream through the solution. Pd(dppf)Cl₂ (6.82 mg, 0.01 mmol) is added, and the mixture is further purged. The reaction mixture is stirred at 100° C. for 2 h. After cooling to ambient temperature, the mixture is diluted with ACN/DMF/H₂O/TFA, filtered, and purified by preparative HPLC (Sunfire C18 column, ACN/water gradient containing 0.1% TFA) to yield the titled compound.

| | |
|---|---|
| C₂₁H₁₉F₄N₇ | (M = 445.4 g/mol) |
| ESI-MS: | 446 [M + H]⁺ |
| Rt (HPLC): | 2.34 min, 2.53 min, |
| | 3.03 min and 4.04 min |
| | (method P) |

### Example 4: 6′-fluoro-3-[(2S,4R)-2-methyl-4-(4-methyl-4H-1,2,4-triazol-3-yl)piperidin-1-yl]-6-(trifluoromethyl)-[2,3′-bipyridine]-4-carbonitrile and

### Example 10: 6′-fluoro-3-[(2R,4R)-2-methyl-4-(4-methyl-4H-1,2,4-triazol-3-yl)piperidin-1-yl]-6-(trifluoromethyl)-[2,3′-bipyridine]-4-carbonitrile

6′-Fluoro-3-[2-methyl-4-(4-methyl-4H-1,2,4-triazol-3-yl)piperidin-1-yl]-6-(trifluoromethyl)-[2,3′-bipyridine]-4-carbonitrile (15.0 mg, 0.03 mmol) is separated by chiral SFC (method P) to afford the desired chiral compounds.

### Example 4

| | |
|---|---|
| C₂₁H₁₉F₄N₇ | (M = 445.4 g/mol) |
| ESI-MS: | 446 [M + H]⁺ |
| Rt (HPLC): | 2.53 min |
| | (method P) |

The enantiomer (6′-fluoro-3-[(2R,4S)-2-methyl-4-(4-methyl-4H-1,2,4-triazol-3-yl)piperidin-1-yl]-6-(trifluoromethyl)-[2,3′-bipyridine]-4-carbonitrile) elutes with a retention time of 2.34 min (method P).

¹H NMR (400 MHz, DMSO-d₆) δ ppm 8.57 (d, J=2.3 Hz, 1H), 8.50 (s, 1H), 8.28-8.34 (m, 2H), 7.36-7.40 (m, 1H), 3.61 (s, 3H), 3.42-3.55 (m, 1H), 3.19-3.26 (m, 1H), 3.06-3.15 (m, 1H), 2.95-3.04 (m, 1H), 1.99 (br d, J=12.4 Hz, 1H), 1.79 (br d, J=12.2 Hz, 1H), 1.44-1.58 (m, 2H), 0.93 (d, J=6.1 Hz, 3H)

### Example 10

| | |
|---|---|
| C₂₁H₁₉F₄N₇ | (M = 445.4 g/mol) |
| ESI-MS: | 446 [M + H]⁺ |
| Rt (HPLC): | 4.04 min |
| | (method P) |

The enantiomer (6′-fluoro-3-[(2S,4S)-2-methyl-4-(4-methyl-4H-1,2,4-triazol-3-yl)piperidin-1-yl]-6-(trifluoromethyl)-[2,3′-bipyridine]-4-carbonitrile) elutes with a retention time of 3.03 min (method P).

¹H NMR (400 MHz, DMSO-d₆) δ ppm 8.57 (d, J=2.3 Hz, 1H), 8.50 (s, 1H), 8.28-8.34 (m, 2H), 7.36-7.40 (m, 1H), 3.61 (s, 3H), 3.42-3.55 (m, 1H), 3.19-3.26 (m, 1H), 3.06-3.15 (m, 1H), 2.95-3.04 (m, 1H), 1.99 (br d, J=12.4 Hz, 1H), 1.79 (br d, J=12.2 Hz, 1H), 1.44-1.58 (m, 2H), 0.93 (d, J=6.1 Hz, 3H)

### Example 5

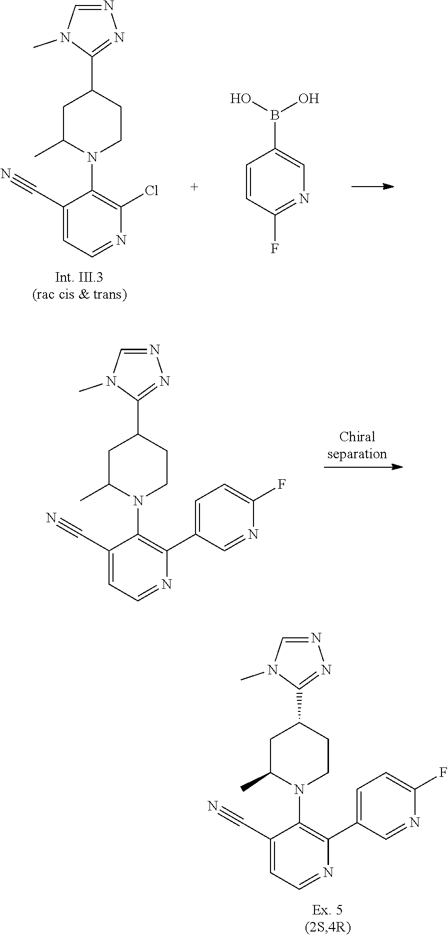 [SMILES: CC1CC(c2nncn2C)CCN1c1c(C#N)ccnc1Cl ; OB(O)c1ccc(F)nc1 ; CC1CC(c2nncn2C)CCN1c1c(C#N)ccnc1-c1ccc(F)nc1 ; C[C@H]1C[C@H](c2nncn2C)CCN1c1c(C#N)ccnc1-c1ccc(F)nc1]

### 6′-Fluoro-3-[2-methyl-4-(4-methyl-4H-1,2,4-triazol-3-yl)piperidin-1-yl]-[2,3′-bipyridine]-4-carbonitrile

To a solution of Int. III.3 (140 mg, 0.44 mmol) and (6-fluoropyridin-3-yl)boronic acid (127 mg, 0.88 mmol) in 1,4-dioxane (2 mL) is added sodium carbonate (2 M in water, 0.66 mL, 1.33 mmol). The resulting mixture is purged by passing an argon stream through the solution. Pd(dppf)Cl₂ (32.3 mg, 0.04 mmol) is added, and the mixture is further purged. The reaction mixture is stirred at 100° C. for 2 h. After cooling to ambient temperature, the mixture is diluted with ACN/H₂O/TFA, filtered, and purified by preparative HPLC (Sunfire C18 column, ACN/water gradient containing 0.1% TFA) to yield the titled compound.

| | |
|---|---|
| C₂₀H₂₀FN₇ | (M = 377.4 g/mol) |
| ESI-MS: | 378 [M + H]⁺ |
| Rt (HPLC): | 0.80 min, 0.94 min |
| | and 1.12 min |
| | (method E) |

### Example 5: 6′-fluoro-3-[(2S,4R)-2-methyl-4-(4-methyl-4H-1,2,4-triazol-3-yl)piperidin-1-yl]-[2,3′-bipyridine]-4-carbonitrile

6′-Fluoro-3-[2-methyl-4-(4-methyl-4H-1,2,4-triazol-3-yl)piperidin-1-yl]-[2,3′-bipyridine]-4-carbonitrile (94.0 mg, 0.25 mmol) is separated by chiral SFC (method E) to afford the desired chiral compound.

| | |
|---|---|
| C₂₀H₂₀FN₇ | (M = 377.4 g/mol) |
| ESI-MS: | 378 [M + H]⁺ |
| Rt (HPLC): | 0.94 min |
| | (method E) |

The enantiomer (6′-fluoro-3-[(2S,4R)-2-methyl-4-(4-methyl-4H-1,2,4-triazol-3-yl)piperidin-1-yl]-[2,3′-bipyridine]-4-carbonitrile) elutes with a retention time of 1.12 min (method E).

¹H NMR (400 MHz, DMSO-d₆) δ ppm 8.61 (d, J=2.2 Hz, 1H), 8.50 (d, J=4.9 Hz, 1H), 8.31-8.39 (m, 2H), 7.76 (d, J=4.9 Hz, 1H), 7.29-7.33 (m, 1H), 5.74-5.77 (m, 1H), 3.68-3.80 (m, 1H), 3.60 (s, 3H), 3.37-3.45 (m, 1H), 3.15-3.24 (m, 1H), 1.90-1.99 (m, 1H), 1.75-1.88 (m, 1H), 1.43-1.59 (m, 2H), 1.08 (d, J=6.7 Hz, 3H)

### Example 6

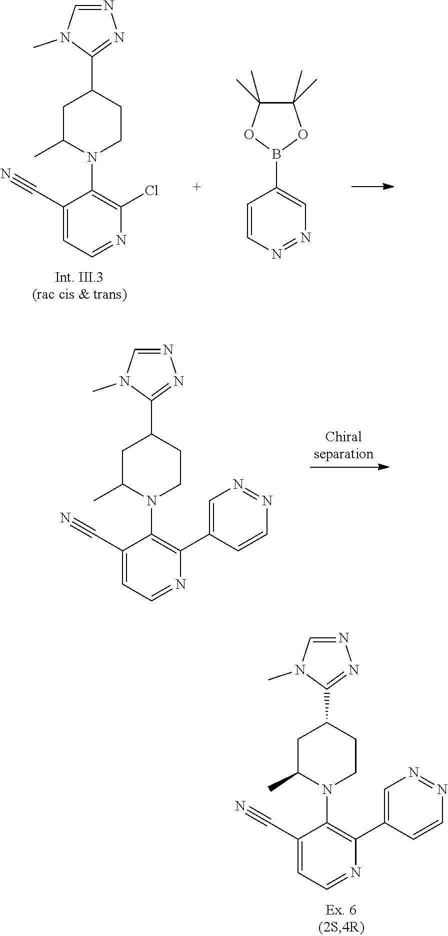

### 3-[2-Methyl-4-(4-methyl-4H-1,2,4-triazol-3-yl)piperidin-1-yl]-2-(pyridazin-4-yl)pyridine-4-carbonitrile

To a solution of Int. III.3 (140 mg, 0.44 mmol) and 4-(4,4,5,5-tetramethyl-1,3,2-dioxaborolan-2-yl)pyridazine (188 mg, 0.88 mmol) in 1,4-dioxane (2 mL) is added sodium carbonate (2 M in water, 0.66 mL, 1.33 mmol). The resulting mixture is purged by passing an argon stream through the solution. Pd(dppf)Cl₂ (32.3 mg, 0.04 mmol) is added, and the mixture is further purged. The reaction mixture is stirred at 100° C. for 2 h. After cooling to ambient temperature, the mixture is diluted with ACN/H₂O/TFA, filtered, and purified by preparative HPLC (Sunfire C18 column, ACN/water gradient containing 0.1% TFA) to yield the titled compound.

| | |
|---|---|
| C₁₉H₂₀N₈ | (M = 360.4 g/mol) |
| ESI-MS: | 361 [M + H]⁺ |
| Rt (HPLC): | 0.92 min, 0.99 min, |
| | 1.89 min and 2.32 min |
| | (method Q) |

### Example 6: 3-[(2S,4R)-2-methyl-4-(4-methyl-4H-1,2,4-triazol-3-yl)piperidin-1-yl]-2-(pyridazin-4-yl)pyridine-4-carbonitrile

3-[2-Methyl-4-(4-methyl-4H-1,2,4-triazol-3-yl)piperidin-1-yl]-2-(pyridazin-4-yl)pyridine-4-carbonitrile (40.0 mg, 0.11 mmol) is separated by chiral SFC (method Q) to afford the desired chiral compound.

| | |
|---|---|
| C₁₉H₂₀N₈ | (M = 360.4 g/mol) |
| ESI-MS: | 361 [M + H]⁺ |
| Rt (HPLC): | 1.89 min |
| | (method Q) |

¹H NMR (400 MHz, DMSO-d₆) δ ppm 9.59 (dd, J=2.0, 1.3 Hz, 1H), 9.38 (dd, J=5.2, 1.1 Hz, 1H), 8.54-8.59 (m, 1H), 8.32 (s, 1H), 8.00-8.04 (m, 1H), 7.84-7.87 (m, 1H), 3.68-3.78 (m, 1H), 3.60 (s, 3H), 3.36-3.49 (m, 2H), 3.15-3.25 (m, 1H), 1.90-1.99 (m, 1H), 1.78-1.89 (m, 1H), 1.40-1.58 (m, 2H), 1.06-1.14 (m, 3H)

### Example 7: 2-{2-tert-butyl-2H-pyrazolo[3,4-b]pyridin-5-yl}-3-[(2S,4R)-2-cyclopropyl-4-(4-methyl-4H-1,2,4-triazol-3-yl)piperidin-1-yl]-6-(trifluoromethyl)pyridine-4-carbonitrile

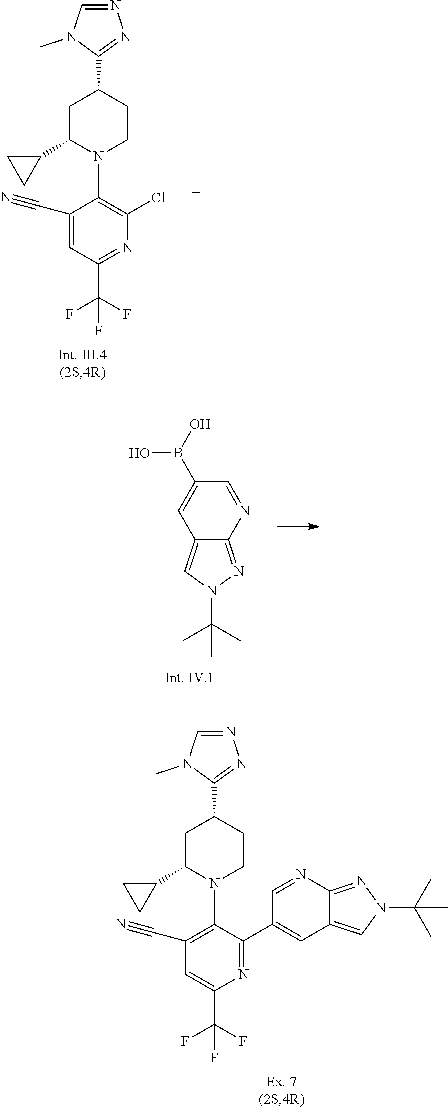 [SMILES: Cn1cnnc1[C@@H]1CCN(c2c(C#N)cc(C(F)(F)F)nc2Cl)[C@H](C2CC2)C1 ; CC(C)(C)n1cc2cc(B(O)O)cnc2n1 ; Cn1cnnc1[C@@H]1CCN(c2c(C#N)cc(C(F)(F)F)nc2-c2cnc3nn(C(C)(C)C)cc3c2)[C@H](C2CC2)C1]

To a solution of Int. III.4 (21.0 mg, 0.05 mmol) and Int. IV.1 as a TFA salt (25.5 mg, 0.08 mmol) in 1,4-dioxane (1 mL) is added potassium carbonate (2 M in water, 89.5 μL, 0.18 mmol). The resulting mixture is purged by passing an argon stream through the solution. Pd(dppf)Cl₂ (3.74 mg, 0.005 mmol) is added, and the mixture is further purged. The reaction mixture is stirred at 95° C. for 3 h. After cooling to ambient temperature, the mixture is diluted with ACN/H₂O/TFA, filtered, and purified by preparative HPLC (XBridge C18 column, ACN/water gradient containing 0.1% NH₃) to yield the titled compound.

| | |
|---|---|
| C₂₈H₃₀F₃N₉ | (M = 549.6 g/mol) |
| ESI-MS: | 550 [M + H]⁺ |
| Rt (HPLC): | 0.70 min (method B) |

¹H NMR (400 MHz, DMSO-d₆) δ ppm 8.91 (d, J=2.0 Hz, 1H), 8.67 (s, 1H), 8.47-8.52 (m, 2H), 8.31 (s, 1H), 3.61 (s, 3H), 3.46-3.54 (m, 2H), 2.97 (tt, J=11.5, 3.5 Hz, 1H), 2.60 (br t, J=8.9 Hz, 1H), 1.77-2.01 (m, 3H), 1.72 (s, 9H), 1.40-1.54 (m, 1H), 0.17-0.38 (m, 2H), 0.01-0.09 (m, 2H), −0.30-−0.22 (m, 1H)

### Example 12: 3-[(2R,4R)-2-methyl-4-(4-methyl-4H-1,2,4-triazol-3-yl)piperidin-1-yl]-2-(pyridazin-4-yl)pyridine-4-carbonitrile

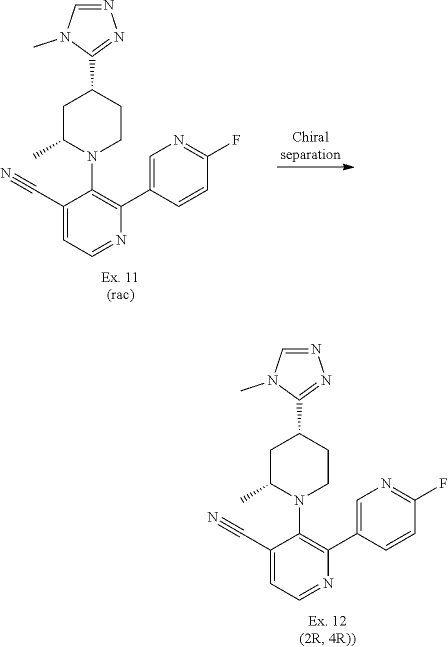 [SMILES: C[C@@H]1C[C@H](c2nncn2C)CCN1c1c(C#N)ccnc1-c1ccc(F)nc1 ; C[C@@H]1C[C@H](c2nncn2C)CCN1c1c(C#N)ccnc1-c1ccc(F)nc1]

Ex. 11 (65.0 mg, 0.21 mmol) is separated by chiral SFC (method R) to afford the desired chiral compound.

| | |
|---|---|
| C₂₀H₂₀N₇ | (M = 377.4 g/mol) |
| ESI-MS: | 378 [M + H]⁺ |
| Rt (HPLC): | 1.01 min |
| | (method R) |

The enantiomer (3-[(2R,4R)-2-methyl-4-(4-methyl-4H-1,2,4-triazol-3-yl)piperidin-1-yl]-2-(pyridazin-4-yl)pyridine-4-carbonitrile) elutes with a retention time of 1.53 min (method R).

¹H NMR (400 MHz, DMSO-d₆) δ ppm 8.73 (d, J=4.9 Hz, 1H), 8.53 (br s, 1H), 8.24-8.34 (m, 2H), 7.91 (d, J=4.8 Hz, 1H), 7.33 (dd, J=8.5, 2.7 Hz, 1H), 3.61 (s, 3H), 3.11-3.24 (m, 2H), 2.90-3.02 (m, 1H), 1.95 (br d, J=12.9 Hz, 1H), 1.82 (br d, J=12.2 Hz, 1H), 1.20-1.59 (m, 3H), 0.81 (d, J=6.1 Hz, 3H)

Analytical data of synthesized examples

| | | Molecular Formula (MW) | |
|---|---|---|---|
| | | ESI-MS | |
| | | HPLC retention time | ¹H NMR (400 MHz, |
| example | Structure | (method) | DMSO-d6): δ in ppm |
| 3 (rac) | 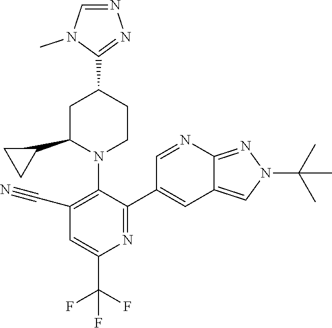 [SMILES: Cn1cnnc1[C@@H]1CCN(c2c(C#N)cc(C(F)(F)F)nc2-c2cnc3nn(C(C)(C)C)cc3c2)[C@@H](C2CC2)C1] | C₂₈H₃₀F₃N₉ (M = 549.6 g/mol) ESI-MS: 550 [M + H]⁺ Rt (HPLC): 0.65 min (method B) | ¹H NMR (400 MHz, DMSO-d₆) δ ppm 8.98 (d, J = 2.3 Hz, 1 H), 8.66 (s, 1 H), 8.52 (d, J = 2.2 Hz, 1 H), 8.32 (s, 1 H), 8.27 (s, 1 H), 3.80 − 3.95 (m, 1 H), 3.60 (s, 3 H), 3.35 − 3.44 (m, 2 H), 2.55 − 2.68 (m, 1 H), 2.02 (br d, J = 10.7 Hz, 1 H), 1.77 − 1.90 (m, 1 H), 1.71 (s, 9 H), 1.62 − 1.72 (m, 3 H), 0.38 − 0.54 (m, 2 H), −0.09 − 0.01 (m, 1 H), −0.24 − −0.17 (m, 1 H) |
| 8 (2S, 4R) | 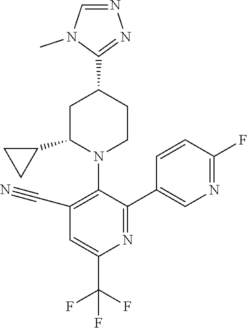 [SMILES: Cn1cnnc1[C@@H]1CCN(c2c(C#N)cc(C(F)(F)F)nc2-c2ccc(F)nc2)[C@H](C2CC2)C1] | C₂₃H₂₁F₄N₇ (M = 471.5 g/mol) ESI-MS: 472 [M + H]⁺ Rt (HPLC): 0.71 min (method S) | ¹H NMR (400 MHz, DMSO-d₆) δ ppm 8.89 (s, 1 H), 8.55 − 8.62 (m, 2 H), 8.30 (td, J = 8.2, 2.4 Hz, 1 H), 7.37 (dd, J = 8.5, 2.7 Hz, 1 H), 3.75 (s, 3 H), 3.39 − 3.54 (m, 2 H), 3.08 − 3.19 (m, 1 H), 2.57 − 2.68 (m, 1 H), 2.01 (br d, J = 12.8 Hz, 2 H), 1.69 − 1.81 (m, 1 H), 1.36 − 1.51 (m, 1 H), 0.01 − 0.29 (m, 4 H), −0.20 − −0.29 (m, 1H) |
| 9 (2R, 4R) | 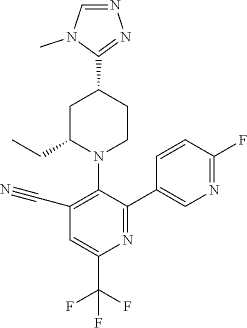 [SMILES: CC[C@@H]1C[C@H](c2nncn2C)CCN1c1c(C#N)cc(C(F)(F)F)nc1-c1ccc(F)nc1] | C₂₂H₂₁F₄N₇ (M = 459.4 g/mol) ESI-MS: 460 [M + H]⁺ Rt (HPLC): 0.64 min (method B) | ¹H NMR (400 MHz, DMSO-d₆) δ ppm 8.56 (d, J = 2.4 Hz, 1 H), 8.50 (s, 1 H), 8.27 − 8.33 (m, 2 H), 7.36 − 7.41 (m, 1 H), 3.61 − 3.63 (m, 3 H), 3.07 − 3.18 (m, 2 H), 2.93 − 3.02 (m, 1 H), 2.06 (br d, J = 11.8 Hz, 1 H), 1.83 (br d, J = 12.2 Hz, 1 H), 1.44 − 1.52 (m, 1 H), 1.21 − 1.26 (m, 1 H), 1.06 − 1.15 (m, 3 H), 0.70 (t, J = 7.4 Hz, 3 H) |
| 11 (rac) | 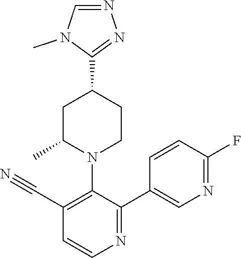 [SMILES: C[C@@H]1C[C@H](c2nncn2C)CCN1c1c(C#N)ccnc1-c1ccc(F)nc1] | C₂₀H₂₀FN₇ (M = 377.4 g/mol) ESI-MS: 378 [M + H]⁺ Rt (HPLC): 0.56 min (method T) | ¹H NMR (400 MHz, DMSO-d₆) δ ppm 8.73 (d, J = 4.8 Hz, 1 H), 8.53 (br s, 1 H), 8.24 − 8.32 (m, 2 H), 7.91 (d, J = 4.9 Hz, 1 H), 7.33 (dd, J = 8.5, 2.8 Hz, 1 H), 3.61 (s, 3 H), 3.11 − 3.22 (m, 2 H), 2.91 − 3.01 (m, 1 H), 2.51 − 2.54 (m, 1H), 1.91 − 1.99 (m, 1 H), 1.79 − 1.86 (m, 1 H), 1.37 − 1.48 (m, 1 H), 1.22 − 1.26 (m, 1 H), 0.81 (d, J = 6.0 Hz, 3 H) |
| 13 (2R, 4S) | 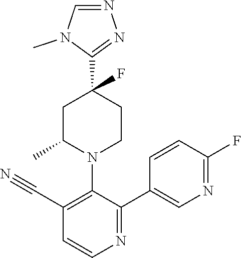 [SMILES: C[C@@H]1C[C@](F)(c2nncn2C)CCN1c1c(C#N)ccnc1-c1ccc(F)nc1] | C₂₀H₁₉F₂N₇ (M = 395.4 g/mol) ESI-MS: 396 [M + H]⁺ Rt (HPLC): 0.77 min (method D) | ¹H NMR (400 MHz, DMSO-d₆) δ ppm 8.76 (d, J = 4.8 Hz, 1 H), 8.50 − 8.56 (m, 2 H), 8.24 − 8.32 (m, 1 H), 7.93 − 7.97 (m, 1 H), 7.30 − 7.36 (m, 1 H), 3.77 − 3.82 (m, 1H), 3.76 (d, J = 1.7 Hz, 3 H), 3.11 − 3.21 (m, 1 H), 2.22 − 2.44 (m, 2 H), 1.71 − 1.90 (m, 1 H), 0.80 (d, J = 6.2 Hz, 3 H) 2H not visible due to overlap with water peak |
| 14 (2R, 4S) | 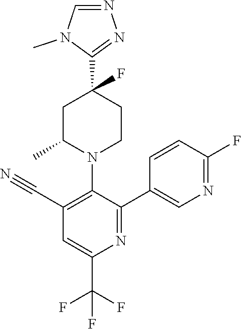 [SMILES: C[C@@H]1C[C@](F)(c2nncn2C)CCN1c1c(C#N)cc(C(F)(F)F)nc1-c1ccc(F)nc1] | C₂₁H₁₈F₅N₇ (M = 463.4 g/mol) ESI-MS: 464 [M + H]⁺ Rt (HPLC): 0.91 min (method D) | ¹H NMR (400 MHz, DMSO-d₆) δ ppm 8.60 (s, 1 H), 8.52 − 8.58 (m, 2 H), 8.31 (td, J = 8.2, 2.5 Hz, 1 H), 7.37 (dd, J = 8.5, 2.7 Hz, 1 H), 3.77 (d, J = 1.7 Hz, 3 H), 3.69 (br s, 1 H), 3.32 − 3.41 (m, 1 H), 3.23 (br d, J = 10.7 Hz, 1 H), 2.38 − 2.47 (m, 1 H), 2.25 (td, J = 12.0, 2.2 Hz, 1 H), 1.81 − 2.11 (m, 2 H), 0.91 (d, J = 6.3 Hz, 3 H) |
| 15 (2R, 4S) | 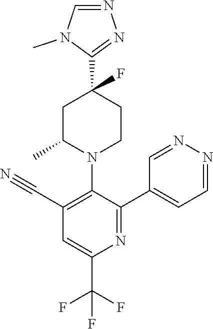 [SMILES: C[C@@H]1C[C@](F)(c2nncn2C)CCN1c1c(C#N)cc(C(F)(F)F)nc1-c1ccnnc1] | C₂₀H₁₈F₄N₈ (M = 446.4 g/mol) ESI-MS: 447 [M + H]⁺ Rt (HPLC): 0.81 min (method D) | ¹H NMR (400 MHz, DMSO-d₆) δ ppm 9.54 (dd, J = 2.2, 1.2 Hz, 1 H), 9.45 (dd, J = 5.3, 1.1 Hz, 1 H), 8.64 (s, 1 H), 8.58 (s, 1 H), 8.01 (dd, J = 5.3, 2.3 Hz, 1 H), 3.76 (d, J = 1.5 Hz, 4 H), 3.36 − 3.45 (m, 1 H), 3.21 − 3.28 (m, 1 H), 2.38 − 2.47 (m, 1 H), 2.20 − 2.30 (m, 1 H), 1.81 − 2.07 (m, 2 H), 0.92 (d, J = 6.3 Hz, 3 H) |
| 16 (2R, 4S) | 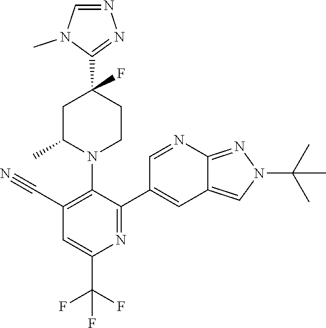 [SMILES: C[C@@H]1C[C@](F)(c2nncn2C)CCN1c1c(C#N)cc(C(F)(F)F)nc1-c1cnc2nn(C(C)(C)C)cc2c1] | C₂₆H₂₇F₄N₉ (M = 5 41.5 g/mol) ESI-MS: 542 [M + H]⁺ Rt (HPLC): 0.91 min (method D) | ¹H NMR (400 MHz, DMSO-d₆) δ ppm 8.91 (d, J = 2.2 Hz, 1 H), 8.70 (s, 1 H), 8.56 (s, 1 H), 8.48 − 8.52 (m, 2 H), 3.74 (d, J = 1.7 Hz, 3 H), 3.55 − 3.70 (m, 1 H), 3.27 − 3.47 (m, 2 H), 2.35 − 2.45 (m, 1 H), 2.21 − 2.30 (m, 1 H), 1.82 − 2.01 (m, 1 H), 1.72 (s, 10 H), 0.91 (d, J = 6.2 Hz, 3 H) |
| 17 (rac) | 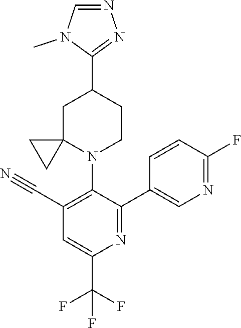 [SMILES: Cn1cnnc1C1CCN(c2c(C#N)cc(C(F)(F)F)nc2-c2ccc(F)nc2)C2(CC2)C1] | C₂₂H₁₉F₄N₇ (M = 457.4 g/mol) ESI-MS: 458 [M + H]⁺ Rt (HPLC): 0.71 min (method S) | ¹H NMR (400 MHz, DMSO-d₆) δ ppm 8.28 (d, J = 16.1 Hz, 3 H), 7.98 (br t, J = 7.0 Hz, 1 H), 7.35 (dd, J = 8.5, 2.5 Hz, 1 H), 3.58 (s, 3 H), 2.52 − 3.02 (m, 3.5H), 0.21 − 2.12 (m, 7.5H). The signals for the cyclopropyl and piperidine ring were found to be doubled due to the presence of rotamers. |

### Assignment of Stereochemistry

Relative Stereochemistry of final compounds was assigned based on NMR of the final compounds or of suitable intermediates. Absolute stereochemistry was assigned based on co-crystal structures of examples 1, 5, 7, 8 and 12 with a human QPCT construct (described below) and extended to the other examples based on biological activity in Assay 1 and Assay 2. The more active enantiomer was assigned the 4R configuration in case R¹ is H or the 4S configuration in case R¹ is F according to formula (I). Co-crystal structures can be obtained as described below.

Structure determination of QPCT-ligand-complexes.

The sequence coding for the human QPCT double mutant Y115E-Y117E with N-terminal His-tag was synthesized by GeneArt and cloned into p-ET17 for expression in E. coli.

Protein sequence:
MRSGHHHHHHGSASAWPEEKNYHQPAILNSSALRQIAEGTSISEMWQND
LQPLLIERYPGSPGSYAARQHIMQRIQRLOADWVLEIDTFLSQTPEGER
SFSNIISTLNPTAKRHLVLACHYDSKYFSHWNNRVFVGATDSAVPCAMM
LELARALDKKLLSLKTVSDSKPDLSLQLIFFDGEEAFLHWSPQDSLYGS
RHLAAKMASTPHPPGARGTSQLHGMDLLVLLDLIGAPNPTFPNFFPNSA
RWFERLQAIEHELHELGLLKDHSLEGRYFQNYSYGGVIQDDHIPFLRRG
VPVLHLIPSPFPEVWHTMDDNEENLDESTIDNLNKILQVFVLEYLHL

The plasmid coding for the QPCT sequence was transformed in E. coli strain BL21(DE3) and the cells were grown in LB medium supplemented with 100 μg/ml ampicillin at 37° C. while shaking. At OD600 of 0.7 the expression was induced with 1 mM IPTG and cells were grown at 20° C. overnight. Cells were harvested by centrifugation, resuspended in lysis buffer (50 mM Tris pH 8.0; 150 mM NaCl, 20 mM imidazole, DNase, Protease inhibitor tablets) and disrupted by sonication. The resulting crude extract was centrifuged (50,000×g for 1 h) and further purified by nickel-affinity chromatography (with 500 mM imidazole in the elution buffer) and size exclusion chromatography (Superdex 75 in 50 mM Tris pH 8.0; 150 mM NaCl). Peak fractions containing highly pure human QPCT were pooled, concentrated to 6 mg/ml and stored at −80° C.

QPCT was crystallized by sitting-drop vapour diffusion at 4° C. Drops were prepared by mixing 75 nl of 6 mg/ml QPCT in 50 mM Tris pH 8.0; 150 mM NaCl with 50 nl of reservoir solution (50 mM ammonium sulfate, 100 mM MES pH 6) and 25 nl seed stock. The seed stock was prepared by collecting several low-diffraction initial crystals of QPCT, transferring them to a tube with 50 μl of fresh reservoir solution and one Seed Bead (Hampton Research) and crushing them by vortexing. 50 μl of reservoir solution were added to the suspension and the seed stock frozen until further use. Prior to setting up crystallization conditions the seed stock was thawed, mixed first 1:1 and then 1:500 with reservoir solution. Crystals diffracting to approx. 1.7 Å appeared within one week. A soaking solution was prepared by dissolving compounds at 5 mM in reservoir solution and added to the drop containing the crystals. After incubation over night at 4° C. crystals were harvested and flash frozen in liquid nitrogen.

Diffraction data was collected on beamline X10SA at the Swiss Light Source (SLS). Data were processed with autoPROC (Vonrhein, C., Flensburg, C., Keller, P., Sharff, A., Smart, O., Paciorek, W., Womack, T. & Bricogne, G. (2011). “Data processing and analysis with the autoPROC toolbox.” Acta Cryst. D 67, 293-302.), structures solved by molecular replacement with PHASER (McCoy A J, Grosse-Kunstleve R W, Adams P D, Winn M D, Storoni L C, Read R J (2007). “Phaser crystallographic software.” J. Appl. Cryst. 40, 658-674) and refined with BUSTER (Bricogne G., Blanc E., Brandl M., Flensburg C., Keller P., Paciorek W., Roversi P, Sharff A., Smart O. S., Vonrhein C., Womack T. O. (2023). BUSTER version 2.11.8. Cambridge, United Kingdom: Global Phasing Ltd.). Manual rebuilding and modelling of the missing atoms into the electron density was performed with Coot (Emsley, P., Lohkamp, B., Scott, W. G., Cowtan, K. (2010). “Features and development of Coot.” Acta Cryst. D 66, 486-5401.).

Analytical HPLC methods

Method A

| time | Vol % water | Vol % | Flow |
|---|---|---|---|
| (min) | (incl. 0.1% TFA) | ACN | [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity; Analytical column: Xbridge (Waters) BEH C18_2.1 × 30 mm_1.7 μm; column temperature: 60° C.

Method B

| time | Vol % water | Vol % | Flow |
|---|---|---|---|
| (min) | (incl. 0.1% NH₃) | ACN | [mL/min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity; Analytical column: XBridge (Waters) BEH C18_2.1 × 30 mm_2.5 μm; column temperature: 60° C.

Method C

| time | Vol % water | Vol % | Flow |
|---|---|---|---|
| (min) | (incl. 0.1% NH₃) | ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Xbridge (Waters) C18_3.0 × 30 mm_2.5 μm; column temperature: 60° C.

Method D

| time | Vol % water | Vol % | Flow |
|---|---|---|---|
| (min) | (incl. 0.1% TFA) | ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Sunfire C18_3.0 × 30 mm_2.5 μm; column temperature: 60° C.

Method E

| | | Vol % MeOH | Flow |
|---|---|---|---|
| time (min) | Vol % scCO₂ | (incl. 20 mM NH₃) | [mL/min] |
| 0.00 | 90 | 10 | 2.0 |
| 4.00 | 90 | 10 | 2.0 |

Device description: Agilent 1260 Infinity II SFC with DAD; Analytical column: Phenomenex Lux ® Cellulose-3_3 × 100 mm_3 μm; column temperature: 40° C.; back pressure: 2175.0 psi

Method F

| | Vol % water | Vol % ACN | Flow |
|---|---|---|---|
| time (min) | (incl. 0.04% TFA) | (incl. 0.02% TFA) | [mL/min] |
| 0.01 | 5 | 95 | 1.5 |
| 0.70 | 95 | 5 | 1.5 |
| 1.16 | 95 | 5 | 1.5 |
| 1.50 | 5 | 95 | 1.5 |
| 1.51 | 5 | 95 | 1.5 |

Device description: Agilent 1200 HPLC MSD:6120; Analytical column: Kinetex C18 LC_2.1 × 30 mm_5 μm; column temperature: 40° C.

Method G

| | Vol % water | Vol % ACN | Flow |
|---|---|---|---|
| time (min) | (incl. 0.04% TFA) | (incl. 0.02% TFA) | [mL/min] |
| 0.01 | 5 | 95 | 1.5 |
| 0.70 | 95 | 5 | 1.5 |
| 1.16 | 95 | 5 | 1.5 |
| 1.50 | 5 | 95 | 1.5 |

Device description: Shimadzu LC-20ADXR MSD:LCMS-2020; Analytical column: Agilent Poroshell SB-C18 3.0 × 30 mm_2.7 μm; column temperature: 40° C.

Method H

| | Vol % water | Vol % ACN | Flow |
|---|---|---|---|
| time (min) | (incl. 0.04% TFA) | (incl. 0.02% TFA) | [mL/min] |
| 0.01 | 10 | 90 | 1.5 |
| 0.80 | 100 | 0 | 1.5 |
| 1.30 | 100 | 0 | 1.5 |

Device description: Shimadzu LC-20ADXR MSD:LCMS-2020; Analytical column: Halo C18 3.0 × 30 mm_5 μm; column temperature: 40° C.

Method I

| | | Vol % IPA | Flow |
|---|---|---|---|
| time (min) | Vol % scCO₂ | (incl. 0.1% IPAm) | [mL/min] |
| 0.0 | 90 | 10 | 2.5 |
| 0.5 | 90 | 10 | 2.5 |
| 3.5 | 50 | 50 | 2.5 |
| 4.5 | 50 | 50 | 2.5 |
| 5.0 | 90 | 10 | 2.5 |

Device description: Waters SFC350; Analytical column: Chiralcel OD-3 4.6 × 150 mm_3 μm; column temperature: 35° C.; back pressure: 2000 psi

Method J

| | | Vol % IPA | Flow |
|---|---|---|---|
| time (min) | Vol % scCO₂ | (incl. 0.1% IPAm) | [mL/min] |
| 0.0 | 95 | 5 | 3.4 |
| 0.2 | 95 | 5 | 3.4 |
| 1.2 | 50 | 50 | 3.4 |
| 2.2 | 50 | 50 | 3.4 |
| 2.6 | 95 | 5 | 3.4 |
| 3.0 | 95 | 5 | 3.4 |

Device description: Waters SFC350; Analytical column: Chiralcel OX-3 4.6 × 50 mm I.D; 3 μm; column temperature: 35° C.; back pressure: 1800 psi

Method K

| | | Vol % EtOH | Flow |
|---|---|---|---|
| time (min) | Vol % scCO₂ | (incl. 0.1% IPAm) | [mL/min] |
| 0.0 | 90 | 10 | 2.5 |
| 0.5 | 90 | 10 | 2.5 |
| 3.5 | 50 | 50 | 2.5 |
| 4.5 | 50 | 50 | 2.5 |
| 5.0 | 0 | 90 | 2.5 |

Device description: Waters SFC350; Analytical column: Chiralpak AD-3 4.6 × 150 mm I.D; 3 μm; column temperature: 35° C.; back pressure: 2000 psi

Method L

| | Vol % water | Vol % ACN | Flow |
|---|---|---|---|
| time (min) | (incl. 0.05% TFA) | (incl. 0.05% TFA) | [mL/min] |
| 0.00 | 100 | 0 | 1.0 |
| 0.40 | 100 | 0 | 1.0 |
| 7.00 | 70 | 30 | 1.0 |
| 10.00 | 10 | 90 | 1.0 |
| 10.01 | 100 | 0 | 1.0 |

Device description: Waters H-Class; Analytical column: XSelect HSS T3 4.6 × 150 mm; 3.5 μm; column temperature: 40° C.

Method N

| | Vol % water | | Flow |
|---|---|---|---|
| time (min) | (incl. 0.1% TFA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Zorbax (Agilent) StableBond C18_3.0 × 30 mm_1.8 μm; column temperature: 60° C.

Method O

| | | Vol % MeOH | Flow |
|---|---|---|---|
| time (min) | Vol % scCO₂ | (incl. 20 mM NH₃) | [mL/min] |
| 0.00 | 85 | 15 | 4.0 |
| 10.0 | 85 | 15 | 4.0 |

Device description: Agilent 1260 Infinity II SFC with DAD and MS; Analytical column: YMC chiral art ® Cellulose SB_4.6 × 250 mm_5 μm; column temperature: 40° C.; back pressure: 2175.0 psi

Method P

| | | Vol % MeOH | Flow |
|---|---|---|---|
| time (min) | Vol % scCO₂ | (incl. 20 mM NH₃) | [mL/min] |
| 0.00 | 85 | 15 | 4.0 |
| 10.0 | 85 | 15 | 4.0 |

Device description: Agilent 1260 Infinity II SFC with DAD and MS; Analytical column: Daicel Chiralpak ® IG_4.6 × 250 mm_5 μm; column temperature: 40° C.; back pressure: 2175.0 psi

Method Q

| | | Vol % EtOH | Flow |
|---|---|---|---|
| time (min) | Vol % scCO₂ | (incl. 20 mM NH₃₎ | [mL/min] |
| 0.00 | 75 | 25 | 2.0 |
| 4.00 | 75 | 25 | 2.0 |

Device description: Agilent 1260 Infinity II SFC with DAD; Analytical column: YMC Chiral Art ® Amylose-C NeO_3 × 100 mm_3 μm; column temperature: 40° C.; back pressure: 2175.0 psi

Method R

| | | Vol % EtOH | Flow |
|---|---|---|---|
| time (min) | Vol % scCO₂ | (incl. 20 mM NH₃) | [mL/min] |
| 0.00 | 80 | 20 | 2.0 |
| 4.00 | 80 | 20 | 2.0 |

Device description: Agilent 1260 Infinity II SFC with DAD; Analytical column: YMC Chiral Art ® Amylose-C NeO_3 × 100 mm_3 μm; column temperature: 40° C.; back pressure: 2175.0 psi

Method S

| | Vol % water | | Flow |
|---|---|---|---|
| time (min) | (incl. 0.1% TFA) | Vol % ACN | [mL/min] |
| 0.00 | 95 | 5 | 1.5 |
| 1.30 | 0 | 100 | 1.5 |
| 1.50 | 0 | 100 | 1.5 |

Device description: Waters Acquity; Analytical column: Sunfire (Waters) C18_3.0 × 30 mm_2.5 μm; column temperature: 60° C.

Method T

| | Vol. % water | | |
|---|---|---|---|
| time (min) | (incl. 0.1 % NH₃) | Vol. % ACN | Flow [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: XBridge (Waters) C18_3.0 × 30 mm_2.5 μm; column temperature: 60° C.

1. A compound of formula (I) 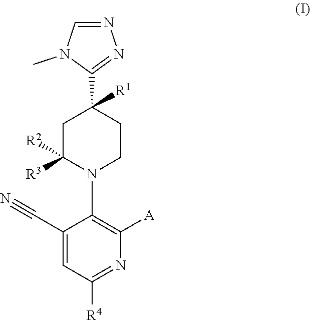
wherein
A is A1a which is a 6-membered mono-heteroaryl ring containing one or two heteroatom members selected from nitrogen;
or A is A1b which is a 9-membered fused bicyclic-heteroaryl ring containing one to four heteroatom members selected from nitrogen;
and wherein A is independently optionally substituted with one or two R⁵;
R¹ is selected from the group R1a, consisting of H and halo;
R² is selected from the group R2a, consisting of H, C₁₋₆-alkyl, F₁₋₉-fluoro-C₁₋₆-alkyl and C₃₋₆-cycloalkyl;
R³ is selected from the group R3a, consisting of H, C₁₋₆-alkyl, F₁₋₉-fluoro-C₁₋₆-alkyl and C₃₋₆-cycloalkyl;
with the proviso that R² and R³ are not both H;
or R² and R³ together with the carbon atom to which they are attached, form a cyclopropyl ring or a cyclobutyl ring; wherein said cyclopropyl or cyclobutyl ring is optionally substituted with one or two F;
R⁴ is selected from the group R4a, consisting of H, H₃C—O, HO—(H₃C)₂C, C₁₋₄-alkyl, C₃₋₆-cycloalkyl, F₁₋₉-fluoro-C₁₋₄-alkyl and F₁₋₉-fluoro-C₃₋₆-cycloalkyl;
R⁵ is selected from the group R5a, consisting of H, halo, C₁₋₆-alkyl and F₁₋₉-fluoro-C₁₋₄-alkyl;
or a salt thereof, particularly a pharmaceutically acceptable salt thereof.

2. The compound of formula (I) according to claim 1, wherein A is selected from the group A2 consisting of 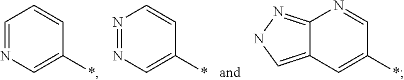
or a salt thereof.

3. The compound of formula (I) according claim 1, wherein R² is H and R³ is selected from the group R3c, consisting of H₃C, H₃CH₂C and cyclopropyl;
or R² and R³ together with the carbon atom to which they are attached, form a cyclopropyl ring; or a salt thereof.

4. The compound of formula (I) according to claim 1, wherein R³ is H and R² is selected from the group R2c, consisting of H₃C, H₃CH₂C and cyclopropyl;
or R² and R³ together with the carbon atom to which they are attached, form a cyclopropyl ring; or a salt thereof.

5. The compound of formula (I) according to claim 1, wherein R⁴ is selected from the group R4c, consisting of H and F₃C; or a salt thereof.

6. The compound of formula (I) according to claim 1, wherein R⁵ is selected from the group R5c, consisting of H, F and (H₃C)₃C; or a salt thereof.

7. The compound of formula (I) according to claim 1 having formula (1-a) 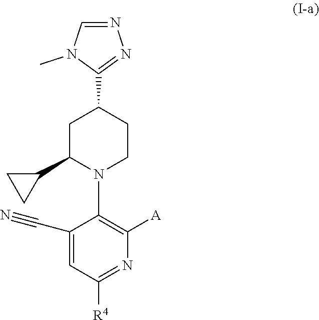
or a salt thereof.

8. The compound of formula (I) according to claim 1 having formula (1-c) 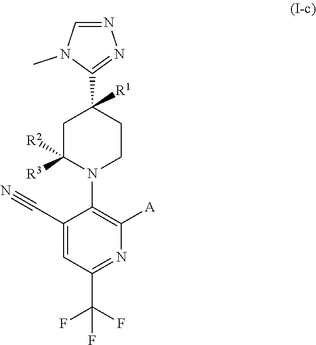
or a salt thereof.

9. The compound of formula (I) according to claim 1, selected from the group consisting of 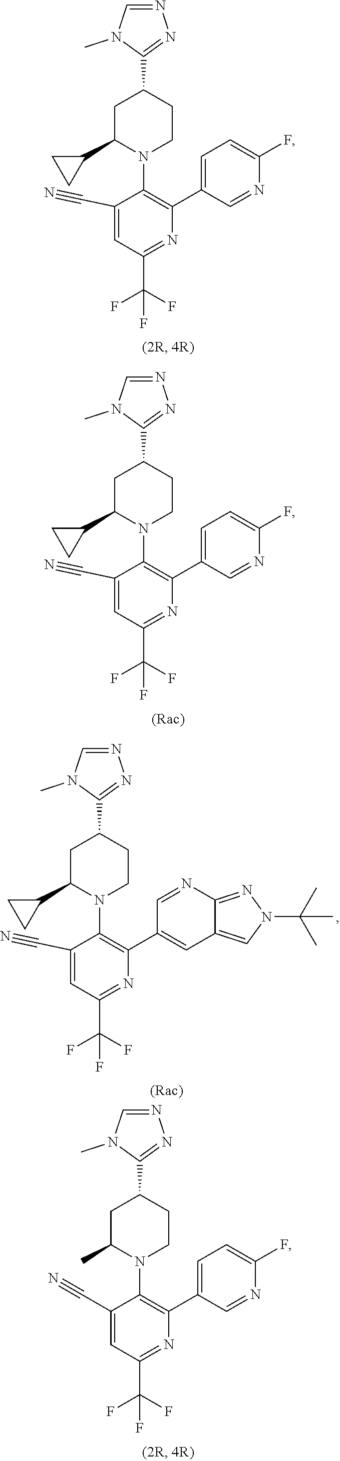 [SMILES: Cn1cnnc1[C@@H]1CCN(c2c(C#N)cc(C(F)(F)F)nc2-c2ccc(F)nc2)[C@@H](C2CC2)C1 ; Cn1cnnc1[C@@H]1CCN(c2c(C#N)cc(C(F)(F)F)nc2-c2ccc(F)nc2)[C@@H](C2CC2)C1 ; Cn1cnnc1[C@@H]1CCN(c2c(C#N)cc(C(F)(F)F)nc2-c2cnc3nn(C(C)(C)C)cc3c2)[C@@H](C2CC2)C1 ; C[C@H]1C[C@H](c2nncn2C)CCN1c1c(C#N)cc(C(F)(F)F)nc1-c1ccc(F)nc1] 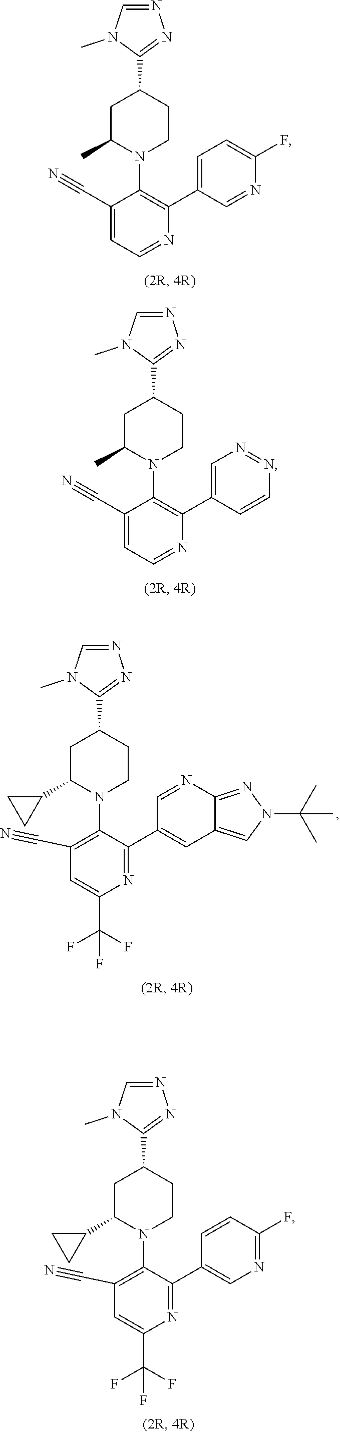 [SMILES: C[C@H]1C[C@H](c2nncn2C)CCN1c1c(C#N)ccnc1-c1ccc(F)nc1 ; C[C@H]1C[C@H](c2nncn2C)CCN1c1c(C#N)ccnc1-c1ccnnc1 ; Cn1cnnc1[C@@H]1CCN(c2c(C#N)cc(C(F)(F)F)nc2-c2cnc3nn(C(C)(C)C)cc3c2)[C@H](C2CC2)C1 ; Cn1cnnc1[C@@H]1CCN(c2c(C#N)cc(C(F)(F)F)nc2-c2ccc(F)nc2)[C@H](C2CC2)C1] 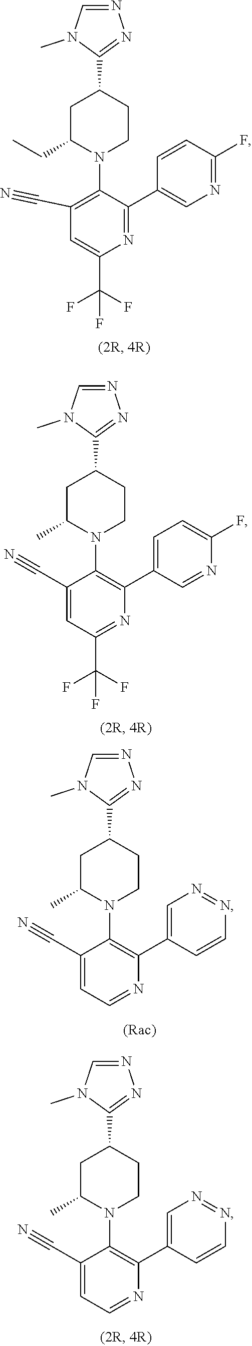 [SMILES: CC[C@@H]1C[C@H](c2nncn2C)CCN1c1c(C#N)cc(C(F)(F)F)nc1-c1ccc(F)nc1 ; C[C@@H]1C[C@H](c2nncn2C)CCN1c1c(C#N)cc(C(F)(F)F)nc1-c1ccc(F)nc1 ; C[C@@H]1C[C@H](c2nncn2C)CCN1c1c(C#N)ccnc1-c1ccnnc1 ; C[C@@H]1C[C@H](c2nncn2C)CCN1c1c(C#N)ccnc1-c1ccnnc1] 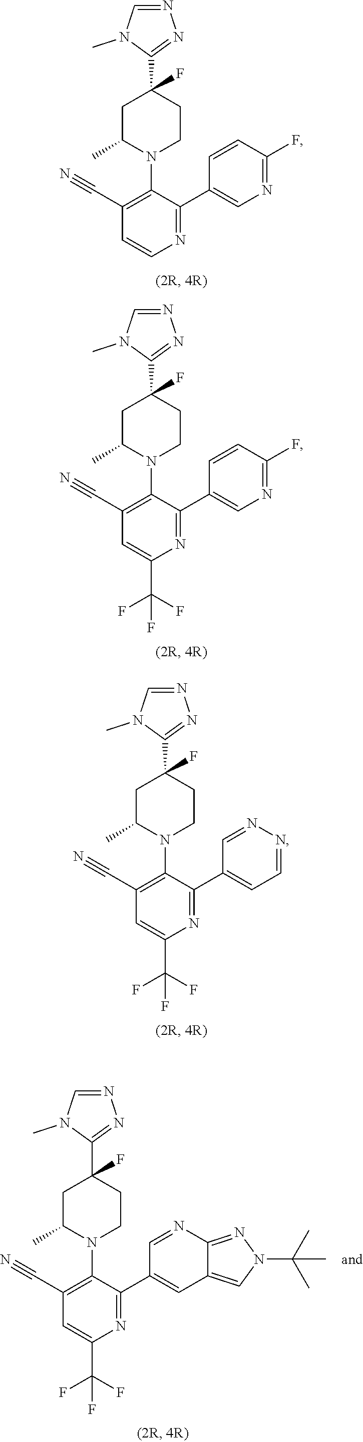 [SMILES: C[C@@H]1C[C@](F)(c2nncn2C)CCN1c1c(C#N)ccnc1-c1ccc(F)nc1 ; C[C@@H]1C[C@](F)(c2nncn2C)CCN1c1c(C#N)cc(C(F)(F)F)nc1-c1ccc(F)nc1 ; C[C@@H]1C[C@](F)(c2nncn2C)CCN1c1c(C#N)cc(C(F)(F)F)nc1-c1ccnnc1 ; C[C@@H]1C[C@](F)(c2nncn2C)CCN1c1c(C#N)cc(C(F)(F)F)nc1-c1cnc2nn(C(C)(C)C)cc2c1] 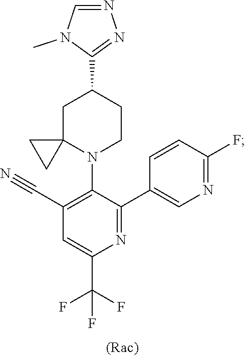 [SMILES: Cn1cnnc1[C@@H]1CCN(c2c(C#N)cc(C(F)(F)F)nc2-c2ccc(F)nc2)C2(CC2)C1]
or a salt thereof.

10. A pharmaceutically acceptable salt of a compound according to claim 1.

11. A pharmaceutical composition comprising one or more compounds according to claim 1, or pharmaceutically acceptable salts thereof, optionally together with one or more inert carriers and/or diluents.

12. A pharmaceutical composition comprising one or more compounds according to claim 1, or pharmaceutically acceptable salts thereof, and one or more additional therapeutic agents, optionally together with one or more inert carriers and/or diluents.

13. The pharmaceutical composition according to claim 12 wherein the one or more additional therapeutic agents are selected from the group consisting of anticancer agents and antifibrotic agents.

14. (canceled)

15. A method for the treatment of cancer or fibrotic disease, in a patient in need thereof, comprising administering a therapeutically effective amount of one or more compounds according to claim 1 or pharmaceutically acceptable salts thereof to the patient.

16. A method for the treatment of cancer, fibrotic diseases, neurodegenerative diseases, atherosclerosis, infectious diseases, or chronic kidney diseases in a patient in need thereof, comprising administering a therapeutically effective amount of one or more compounds according to claim 1 or pharmaceutically acceptable salts thereof to the patient.
